SEQUENCE LISTING (175 sequences)

SEQ ID NO: 1 - DNA, 2022 bp, Arabidopsis thaliana
atggcgctaaagcgagggctatctggagttaaccggattagaggaagtggtggtggatctcgatctgtgcttgtgcttctcatatttttctgtgtttttgcacctctttgcttctttgttggccgaggagtgtatatcgattcctcaaatgattattcaattgtttctgtgaagcagaatcttgactggagagaacgtttagcaatgcaatctgttagatctcttttctcgaaagagatactagatgttatagcaaccagcacagctgatttgggtcctcttagccttgattcttttaagaaaaacaatttgtctgcatcatggcggggaaccggagtagacccctcctttagacattctgagaatccagcaactcctgatgtcaaatctaataacctgaatgaaaaacgtgacagcatttcaaaagatagtatccatcagaaagttgagacacctacaaagattcacagaaggcaactaagagagaaaaggcgtgagatgcgggcaaatgagttagttcagcacaatgatgacacgattttgaaactcgaaaatgctgccattgaacgctctaagtctgttgattctgcagtccttggtaaatacagtatttggagaagagaaaatgagaatgacaactctgattcaaatatacgcttgatgcgggatcaagtaataatggctagagtctatagtgggattgcaaaattgaaaaacaagaacgatttgttacaagaactccaggcccgacttaaggacagccaacgggttttgggggaagcaacatctgatgctgatcttcctcggagtgcgcatgagaaactcagagccatgggtcaagtcttggctaaagctaagatgcagttatatgactgcaagctggttactggaaagctgagagcaatgcttcagactgccgacgaacaagtgaggagcttaaagaagcagagtacttttctggctcagttagcagcaaaaaccattccaaatcctatccattgcctatcaatgcgcttgactatcgattactatcttctgtctccggagaaaagaaaattccctcggagtgaaaacctagaaaaccctaatctttatcattatgccctcttttccgacaatgtattagctgcatcagtagttgttaactcaaccatcatgaatgccaaggatccttctaagcatgtttttcaccttgtcacggataaactcaatttcggagcaatgaacatgtggttcctcctaaacccacccggaaaggcaaccatacatgtggaaaacgtcgatgagtttaagtggctcaattcatcttactgtcctgtccttcgtcagcttgaatctgcagcaatgagagagtactattttaaagcagaccatccaacttcaggctcttcgaatctaaaatacagaaacccaaagtatctatccatgttgaatcacttgagattctacctccctgaggtttatcccaagctgaacaaaatcctcttcctggacgatgacatcattgttcagaaagacttgactccactctgggaagttaacctgaacggcaaagtcaacggtgcagtcgaaacctgtggggaaagtttccacagattcgacaagtatctcaacttttcgaatcctcacattgcgaggaacttcaatccaaatgcttgtggatgggcttatggaatgaacatgttcgacctaaaggaatggaagaagagagacatcactggtatataccacaagtggcaaaacatgaatgagaacaggacactatggaagctagggacattgccaccaggattaataacattctacggattaacacatcccttaaacaaggcgtggcatgtgctgggacttggatataacccgagtatcgacaagaaggacattgagaatgcagcagtggttcactataacgggaacatgaaaccatggttggagttggcaatgtccaaatatcggccgtattggaccaagtacatcaagtttgatcacccatatcttcgtcgttgcaaccttcatgaataa

SEQ ID NO: 2 - protein, 673 aa, Arabidopsis thaliana
MALKRGLSGVNRIRGSGGGSRSVLVLLIFFCVFAPLCFFVGRGVYIDSSNDYSIVSVKQNLDWRERLAMQSVRSLFSKEILDVIATSTADLGPLSLDSFKKNNLSASWRGTGVDPSFRHSENPATPDVKSNNLNEKRDSISKDSIHQKVETPTKIHRRQLREKRREMRANELVQHNDDTILKLENAAIERSKSVDSAVLGKYSIWRRENENDNSDSNIRLMRDQVIMARVYSGIAKLKNKNDLLQELQARLKDSQRVLGEATSDADLPRSAHEKLRAMGQVLAKAKMQLYDCKLVTGKLRAMLQTADEQVRSLKKQSTFLAQLAAKTIPNPIHCLSMRLTIDYYLLSPEKRKFPRSENLENPNLYHYALFSDNVLAASVVVNSTIMNAKDPSKHVFHLVTDKLNFGAMNMWFLLNPPGKATIHVENVDEFKWLNSSYCPVLRQLESAAMREYYFKADHPTSGSSNLKYRNPKYLSMLNHLRFYLPEVYPKLNKILFLDDDIIVQKDLTPLWEVNLNGKVNGAVETCGESFHRFDKYLNFSNPHIARNFNPNACGWAYGMNMFDLKEWKKRDITGIYHKWQNMNENRTLWKLGTLPPGLITFYGLTHPLNKAWHVLGLGYNPSIDKKDIENAAVVHYNGNMKPWLELAMSKYRPYWTKYIKFDHPYLRRCNLHE

SEQ ID NO: 3 - skipped

SEQ ID NO: 4 - protein, 644 aa, Populus trichocarpa
MALKRGFSISGLNKNRRGGSRLPIVVVIFFCVLSPLIFFVGRGLYTTSSSTAFELERTAGLATCEIDFLKRVIGIDSSVEDNAASEPNQTATVVKQEAPKGKEDNISDDDSRSGDTPAKLARRFMQQLREKRREKRAVELLRQDDEAIARLESAAIERSKLVDGAVLGKYSIWRKEMDSENSDSTVRLMRDQMIMARVYLSIAKMKRKLDLLQELQTRIKESQRVLGDSLADSDLHPSAPEKIKAMGQVLSKARELLYDCKLVTGKLRAMLQTADEQVRSLKKQSTFLSQLAAKTVPNGIHCLSMRLTIDYYLLPLEKRKFPRSENLENPNLYHYALFSDNVLAASVVVNSTIMNAKDSSKHVFHLVTDKLNFGAMNMWFLLNPPGKATIHVENVDEFKWLNSSYCPVLRQLESAAMKEYYFKANHPTSLSSGSSNLKYRNPKYLSMLNHLRFYLPEVYPKLDKILFLDDDIVVQKDLTKLWSVDLHGKVNGAVETCGESFHRFDKYLNFSNPHIAKNFDPNACGWAYGMNIFDLKVWKKKDITGIYHKWQNMNEDRVLWKLGTLPPGLITFYNLTNPLEKTWHVLGLGYNPSIDRSEIESAAVVHYNGNMKPWLELAMTKYRPYWTKYIKYDHPYLRNCNLSE

SEQ ID NO: 5 - skipped

SEQ ID NO: 6 - protein, 687 aa, Populus trichocarpa
MALKRGLSSSGVNKNRSGGGGGSRLPIILVIFFCFLSPLIFFVGRRLIITSSSDQNNNNNAVGSGKQQLDWRERLALQHVKPLFSKEVIDVIASSTADLGPLSLDSSRKNKLSASWKVIGGETPVDNKAASETNQTATVVKQEASKGKVDNISEDNARSGDTPAKLARRQLREKRREKRVAELLRQDDEATARLENAAIERSKLVDGAVLGKYSIWRKEMDNENSDSTVRLMRDQMIMARVYLSIAKMKNKRDLLQELQTRLKESQRALGESSADSDLHPSAPGKLKAMGQVLSKAREQLYDCKLVTGKLRAMLQTADEQVRSLKKQSTFLSQLAAKTVPNGIHCLSMRLTIDYYLLPLEKRKFPRSEDLENPNLYHYALFSDNVLAASVVVNSTIMNAKDSSKHVFHLVTDKLNFGAMNMWFLLNPPGKATIHVENVDEFKWLNSSYCPVLRQLESAAMKEYYFKANHPTSLSSGSSNLKYRNPKYLSMLNHLRFYLPQVYPKLDKILFLDDDIVVQKDLTKLWSVDLNGKVNGAVETCGESFHRFDKYLNFSNPHIARHFDPNSCGWAYGMNIFDLKVWKKKDITGIYHKWQNMNEDRVLWKLGTLPPGLITFYNLTHPLQKSWHVLGLGYNPSIDRSEIENAAVVHYNGNMKPWLELAMTKYRPYWTKYIKYDHPYLRNCNLSE

SEQ ID NO: 7 - DNA, 1587 bp, Arabidopsis thaliana
atgactgatgcttgttgtttgaagggaaacgaggacaaaatggttcctcgttttggtcatggaacctggataggaaaagcatttaatgatacaccagagatgttgcatgaaaggagtctgagacaggaaaaaagattggaaagggctaatgagctgatgaatgatgatagtctgcaaaagcttgagacggcagccatggcacgttccagatctgtcgattctgcaccactaggaaactacaccatttggaaaaatgaataccggaggggcaagagttttgaagatatgttacgtttgatgcaagatcaaatcatcatggcacgagtttacagtggacttgcaaagtttacaaacaatctcgccttgcaccaagagatagaaacacaactaatgaaactagcttgggaggaagaatctactgatattgatcaggagcagagagtacttgacagtataagagacatgggacaaatactggctagagcacacgagcagctatatgaatgcaagttggtgacaaataagttgagagcaatgctacaaacagttgaagatgaactcgaaaacgagcagacttatataacgttcttgactcagctagcttccaaggcactaccagatgctatccactgcttgaccatgcgcttgaatctagagtatcatctcctgcctttaccgatgagaaattttccaaggagggagaatttggagaatccaaaactttaccactacgctctcttctctgataatgtactggctgcatcagttgttgtcaactccacagtcatgaatgcacaggatccttcaaggcatgttttccaccttgtgactgataagctcaactttggagcaatgagtatgtggtttctgttgaaccctcctggagaagcgaccatccatgtccaaaggtttgaagattttacttggctcaactcatcttactctccagttttgagtcagctcgagtcagcagctatgaagaagttctacttcaagacagcgaggtctgaatcagttgaatcaggctcagaaaacctcaagtaccggtacccgaaatacatgtcaatgcttaaccacctgaggttctacatccctaggatcttcccaaagttggagaaaatcttgtttgttgacgatgatgtggttgttcagaaggatttaactcccctatggtccattgatcttaaagggaaagtgaatgaaaactttgatcccaagttctgcggatgggcttatgggatgaacatcttcgacctgaaagaatggaagaagaacaacattacagaaacttatcacttttggcaaaacctgaacgaaaaccggactctatggaaactaggaacattgccaccagggctcataacgttctacaatctgacacaaccacttcagagaaaatggcacttacttggactgggttatgataaaggaatcgatgtcaagaagattgaaagatcagctgttatacattacaatggacacatgaaaccatggacagagatggggataagcaagtatcagccatattggacgaagtacaccaattttgaccatccttacatctttacttgcaggctgtttgagtga

SEQ ID NO: 8 - protein, 528 aa, Arabidopsis thaliana
MTDACCLKGNEDKMVPRFGHGTWIGKAFNDTPEMLHERSLRQEKRLERANELMNDDSLQKLETAAMARSRSVDSAPLGNYTIWKNEYRRGKSFEDMLRLMQDQIIMARVYSGLAKFTNNLALHQEIETQLMKLAWEEESTDIDQEQRVLDSIRDMGQILARAHEQLYECKLVTNKLRAMLQTVEDELENEQTYITFLTQLASKALPDAIHCLTMRLNLEYHLLPLPMRNFPRRENLENPKLYHYALFSDNVLAASVVVNSTVMNAQDPSRHVFHLVTDKLNFGAMSMWFLLNPPGEATIHVQRFEDFTWLNSSYSPVLSQLESAAMKKFYFKTARSESVESGSENLKYRYPKYMSMLNHLRFYIPRIFPKLEKILFVDDDVVVQKDLTPLWSIDLKGKVNENFDPKFCGWAYGMNIFDLKEWKKNNITETYHFWQNLNENRTLWKLGTLPPGLITFYNLTQPLQRKWHLLGLGYDKGIDVKKIERSAVIHYNGHMKPWTEMGISKYQPYWTKYTNFDHPYIFTCRLFE

SEQ ID NO: 9 - DNA, 2043 bp, Arabidopsis thaliana
atgacgacgttctctacatgcgccgcctttttatcgctggtagtagtgctacatgctgttcatgtcggtggagccattttagagtcacaagcaccccacagagaacttaaagcttatcgtccgctgcaagataataatctacaggaggtgtatgcttcctcagctgctgcagtgcactacgatccagatctgaaagatgtgaacatagttgcgacatacagtgaccattacggcaatatacgccttggtagggtgaaaatgggggatctttcaccttcttgggttttggagaatcctgcctatcaagttagccgcaaaacaaaaggttcgcagctagttataccacgggattcatttcaaaatgatactggaatggaagataatgcaagccattctacaactaatcagactgatgaaagcgaaaatcagtttccaaacgtggattttgcaagcccagcaaaactgaagcggcagattttacgtcaggaaaggagaggtcaacgaactttagagctgatccgacaagaaaaggaaactgatgagcagatgcaagaagcagccattcagaagtcaatgagctttgaaaactcagtcatagggaaatacagtatatggaggagagactatgagagcccaaatgctgatgctatcttgaagcttatgagagaccagatcataatggcaaaagcatatgcaaatattgccaaatcaaaaaatgtaaccaatctgtacgttttcttgatgcagcagtgtggagaaaataaacgtgttataggtaaagcaacctctgatgctgaccttccttcaagcgctcttgatcaagcaaaagccatgggccatgcactctctcttgcaaaagacgagttatatgactgccatgaacttgcaaaaaagttccgggccatccttcagtccactgaacgcaaagtagatggactgaagaaaaagggaaccttcttaattcagctagctgccaaaacatttcccaagccattgcattgcctgagtctgcagctagcggcagactattttattctaggtttcaatgaagaggatgcagtgaaagaggatgtcagtcaaaagaagcttgaagatccttcgctctatcactatgcgatcttttcggataacgttctggctacatcagtggtggtgaactccactgtcttgaatgcaaaggaaccgcagaggcatgtgttccatatagtaactgacaaactgaattttggtgcaatgaagatgtggtttcgcatcaatgctcctgctgatgcgacgattcaagttgaaaacataaatgatttcaagtggctgaactcctcttactgctctgttctacggcagcttgaatctgcaaggctgaaagaatactatttcaaagcaaatcatccttcatcaatctcagctggcgcagataatctaaagtaccgcaacccaaagtatctatcgatgctgaatcatctcagattctaccttcctgaggtttatccgaagctggagaagattctgtttctagacgatgacattgtggtgcagaaggacctggcaccactatgggaaatagacatgcaaggaaaagtgaatggtgcggtggagacgtgcaaggagagcttccacagatttgacaagtacctcaacttctcaaatccaaagatttcagagaattttgacgctggtgcttgtgggtgggcatttgggatgaatatgtttgacctgaaagagtggaggaaacggaacattacagggatatatcactattggcaagacttgaatgaagacagaacactgtggaagctgggatcgttgccaccggggctgataacattttacaacctgacgtatgcaatggataggagctggcacgtactagggctgggatatgacccagcgctaaaccaaacagcaatagagaatgcagcggtagtgcattacaatgggaactacaagccatggctgggtttagcattcgccaagtacaaaccgtactggtccaagtacgttgagtacgacaacccttatctccgacggtgcgacatcaatgaatga

SEQ ID NO: 10 - protein, 680 aa, Arabidopsis thaliana
MTTFSTCAAFLSLVVVLHAVHVGGAILESQAPHRELKAYRPLQDNNLQEVYASSAAAVHYDPDLKDVNIVATYSDHYGNIRLGRVKMGDLSPSWVLENPAYQVSRKTKGSQLVIPRDSFQNDTGMEDNASHSTTNQTDESENQFPNVDFASPAKLKRQILRQERRGQRTLELIRQEKETDEQMQEAAIQKSMSFENSVIGKYSIWRRDYESPNADAILKLMRDQIIMAKAYANIAKSKNVTNLYVFLMQQCGENKRVIGKATSDADLPSSALDQAKAMGHALSLAKDELYDCHELAKKFRAILQSTERKVDGLKKKGTFLIQLAAKTFPKPLHCLSLQLAADYFILGFNEEDAVKEDVSQKKLEDPSLYHYAIFSDNVLATSVVVNSTVLNAKEPQRHVFHIVTDKLNFGAMKMWFRINAPADATIQVENINDFKWLNSSYCSVLRQLESARLKEYYFKANHPSSISAGADNLKYRNPKYLSMLNHLRFYLPEVYPKLEKILFLDDDIVVQKDLAPLWEIDMQGKVNGAVETCKESFHRFDKYLNFSNPKISENFDAGACGWAFGMNMFDLKEWRKRNITGIYHYWQDLNEDRTLWKLGSLPPGLITFYNLTYAMDRSWHVLGLGYDPALNQTAIENAAVVHYNGNYKPWLGLAFAKYKPYWSKYVEYDNPYLRRCDINE

SEQ ID NO: 11 - skipped

SEQ ID NO: 12 - protein, 655 aa, Populus trichocarpa
MEEQRRRRRRRFWTSSSLALLLIFFMEHDASSVAGHGVQSDEMDINIIATYSDTSGAVRTSRVKMSDLSPSWVLENPADKNHDQPKTSQRLEDSSKAGATHEDDVLHSARDHQYGEGGIPSSWKLPMSPVKLQRQTARKDRRVLRTSVLIQQDKGAADSQTEATAFIWSKSLDTSIKGKYSIWRRDFDSPNSDSTLKLMRDQIIMAKAYANIAKSNNVTTLYNSLMKQSRESQLAIGEAMSDAELHPSALVQAKAMGHVLSIAKDQLYECPTMSRKLRAMLQLNEENVNALKKKSAFLIQLAAKTIPKPLHCLPLQLAADYFLYGYQNKKYLDKEKVQDPSLFHYAIFSDNVLATSVVINSTVQHAKDPQKHVFHIVTDKLNFAAMKMWFIVNPPAKATVQVENIDDFKWLNASYCSVLRQLESARIKEYYFKANHPSSLASGADNLKYRNPKYLSMLNHLRFYLPEVYPKLDKILFLDDDIVVQKDLTPLWSIDLQGMVNGAVETCKESFHRFDKYLNFSNPKIYNNFDPNACGWAFGMNMFDLKQWKRSNITGIYHHWQDLNEDRTLWKLGSLPPGLITFYNLTYPLDRSWHVLGLGYDPALNQTEIENAAVVHYNGNYKPWLDLAVAKYKPYWSRYVQYDNPYLKQCNIVEE

SEQ ID NO: 13 - DNA, 1851 bp, Arabidopsis thaliana
atgatggtgaagcttcgcaatcttgttcttttcttcatgctcctcaccgtcgttgctcatatccttctctacaccgatcccgctgcctccttcaagacccccttttctaaacgcgatttcctcgaggacgtaaccgccttgactttcaattccgatgagaatcgtttgaatcttcttcctcgggaatctcccgctgtgctcagaggaggactcgtcggtgctgtctattccgataagaattcacggcggctagaccaattgtctgctcgagttctttccgccaccgacgatgatactcactcacatactgacatttccatcaaacaagtcactcatgatgcagcctcagactcgcatattaatagggaaaatatgcatgttcaattgacccaacaaacctctgaaaaagttgatgagcaaccagagcctaatgcttttggagctaagaaagatactggaaacgtgttgatgcctgatgctcaagtgaggcatcttaaagatcagcttattagggcaaaggtttatctttcccttccatctgcaaaggccaatgctcattttgtgagagagcttcgactccgtattaaagaagttcaacgggcacttgcagatgcctccaaggattcggatctgccaaagactgctatagaaaagctaaaagcaatggagcaaacactggccaaaggcaagcagatccaagatgactgttctacagtggtcaagaagctacgtgctatgctccactccgcagatgagcagctacgggtccataagaagcaaaccatgtttttgactcaattgactgctaagaccattcctaaaggacttcactgccttcctctgcgcctcactacagactattatgctttaaattcatctgaacaacaatttccaaatcaggagaaactagaagatactcagctgtatcactatgcccttttctctgataatgttttggctacgtcagttgttgttaactctaccataaccaatgcaaagcatcccttaaagcatgtcttccacatcgtcacagacagactcaattatgcggcaatgaggatgtggttcctggacaatccacctggcaaagccaccatccaggttcagaatgttgaagaatttacatggctgaattcaagctacagtcccgttctcaaacagcttagttctagatcgatgatagattattacttcagagcccaccatacaaattcagacaccaacttgaagttccggaatccaaaatacttatcgatccttaatcatcttcgtttttacttgcctgagatctttcccaagctcagcaaagtgctcttcttggatgatgatatagttgtgcagaaggacctttctggtctttggtcagttgatctgaaaggtaatgttaacggtgctgtagagacgtgtggggaaagctttcatcgctttgaccgttatctgaacttctcaaatccactcatttccaagaactttgaccctcgagcttgtggttgggcgtatggtatgaatgtctttgatctggatgaatggaagaggcaaaacatcacagaagtttatcatcgatggcaggatctgaatcaagaccgagaattgtggaagctagggacgttgccgcctggtctaatcacattttggagacgaacatatccgctagaccggaaatggcacatactagggcttggatacaacccgagtgtgaaccaaagggatattgagagggcagccgtgatacactataatggcaacctcaaaccatggctagagattgggattccaagatacagaggcttctggtcaaagcatgtagactatgagcacgtttatctcagagaatgcaacatcaatccttag

SEQ ID NO: 14 - protein, 616 aa, Arabidopsis thaliana
MMVKLRNLVLFFMLLTVVAHILLYTDPAASFKTPFSKRDFLEDVTALTFNSDENRLNLLPRESPAVLRGGLVGAVYSDKNSRRLDQLSARVLSATDDDTHSHTDISIKQVTHDAASDSHINRENMHVQLTQQTSEKVDEQPEPNAFGAKKDTGNVLMPDAQVRHLKDQLIRAKVYLSLPSAKANAHFVRELRLRIKEVQRALADASKDSDLPKTAIEKLKAMEQTLAKGKQIQDDCSTVVKKLRAMLHSADEQLRVHKKQTMFLTQLTAKTIPKGLHCLPLRLTTDYYALNSSEQQFPNQEKLEDTQLYHYALFSDNVLATSVVVNSTITNAKHPLKHVFHIVTDRLNYAAMRMWFLDNPPGKATIQVQNVEEFTWLNSSYSPVLKQLSSRSMIDYYFRAHHTNSDTNLKFRNPKYLSILNHLRFYLPEIFPKLSKVLFLDDDIVVQKDLSGLWSVDLKGNVNGAVETCGESFHRFDRYLNFSNPLISKNFDPRACGWAYGMNVFDLDEWKRQNITEVYHRWQDLNQDRELWKLGTLPPGLITFWRRTYPLDRKWHILGLGYNPSVNQRDIERAAVIHYNGNLKPWLEIGIPRYRGFWSKHVDYEHVYLRECNINP

SEQ ID NO: 15 - skipped

SEQ ID NO: 16 - protein, 665 aa, Populus trichocarpa
MNAVSFSHTSTTKVFSGILTTMRMRNLVMGLLFLTVLSPILLYTDKLSSSFTPSTSKQEDVNAFTLPTDTRHLNVLPQEESSTVIKEPIGIVYTDHINSSSNTILTEKDSQLPDAREHKYARVLSATDDEGHSQTDNIIKQIIQTTNQEEEESQSDNGSDQESQQKTQVQLEQQSAVNSGDDDEKDALLTETNKQTDQTAMPDARVRQLRDQLIKARVYLSLPATKNNPHFTRELRMRVKEVQRVLVDATKDSDLPKNAYAKLNAMDQLLEKGKQMQDDCATMVKKLRAMLHSTEEQLRVHKKQTMFLTQLTAKTLPKGLHCLPLRLTTEYYNLNSTEQQFPNQEKLDDPSLHHIALFSDNVLAAAVVVNSTITNSKLTYPQHPSKLVFHIVSDRLNYAAMRMWFLVNPPGVATIQVQNIEEFTWLNSSYSPVLKQLGSRSMIDYYFRAARASSDSNLKYRNPKYLSILNHLRFYLPEIFPKLNKVLFLDDDIVVQKDLTGLWSLDLKGNVNGAVETCGENFHRFDRYLNFSNPHISKNFDPRACGWAYGMNIFDLKEWKRQNITDVYHTWQKLNHDRQLWKLGTLPPGLITFWKRTHPLDRRWHVLGLGYNPNVSQREIERAAVIHYNGNMKPWLEIGIPKYRSNWAKYVDYDHAYLRECNINP

SEQ ID NO: 17 - skipped

SEQ ID NO: 18 - protein, 648 aa, Populus trichocarpa
MRLRNLVFGLLSLSVLAPILLYIDSFSSFTPSFKQEFLEDVTALILPADTSNLNVLPQDESSAVLKEPIGILYTDNHSKTILTDKGRALSATDEDAQSRKDDIIKQVIQSANQEKEETRTDRGADQESHQLKQQSALNSDKVGEKDALLTKTNKQTDQSPMPAAWERQLRDRLIKASVYLSLPATKNNRRFTRELRMRIKEVQRVLGDAIKDSDMPKNAYEKWKAMDQLLEKGKQMQYESANEVKKLRAMLHSTEEQLRVHKKQTMSFATMVEKLRAMLHSTEEQLQVHKKQTMFLTQLTAKTLPKGLHCLPLRLTTEYYNLNSSEQQFPNQEILDNPLLHHIALFSDNVLAAAVVVNSTVTNSKHPSKLVFHLVSDRLSYAAMRMWFLVNPPGKATIQVQNIDEFTWLNSSYSPVLKQLHSQSMIDYYFRAHSANSDSNLKYRNPKYLSILNHLRFYLPEIFPKLNKVLFLDDDIVVQKDLTGLWSLDLKGKVNGAVETCRESFHRFDTYLNFSNPLISNNFDPRACGWAYGMNLFDLEEWKRQNITDVYHSWQKLNHDRQLWKLGTLPPGLITLWKRTHPLDRRWHVLGLGYNPNVSQIEIERGAVIHYNGNMKPWLEIGIPKYRKYWAKYVDYVNVYLRECNINP

SEQ ID NO: 19 - DNA, 1833 bp, Arabidopsis thaliana
atgaatcaagttcgtcgttggcagaggattctgatcctctcgctgctattgttatctgttttagctccgattgttttcgtttcgaatcggctcaagagcatcacttccgtcgatagaggagaattcattgaagaattatccgacattacagataagaccgaggatgaacttagacttactgctattgaacaggacgaagaaggcttgaaggagcctaaacgtattctgcaggatcgagattttaattctgtggttttgtcaaattcctctgataaaagtaatgatactgtgcagtctaatgagggagaccaaaaaaactttctctcagaagttgataagggaaataatcacaaaccaaaggaggaacaagcagtttcacagaaaaccacagtaagctcgaatgcggaggtgaaaatttcagcaagagatattcaacttaatcataaaacggaattccgacccccttcaagtaagagtgaaaagaatacaagggttcaacttgaaagagcaacagatgagagggtaaaggagatcagagacaaaattatccaagcgaaagcctatctgaatttggccctacctgggaataactcccaaatcgtaaaggagttgagagttcgaacgaaagagctggaacgggctactggtgatactaccaaggataaatatttgccaaagagctctcctaacagattgaaggccatggaagttgcgttatacaaggtcagccgtgcctttcacaactgccctgccattgctaccaaactccaagccatgacttataaaaccgaagaacaagctcgggcgcagaagaaacaagcagcatatttaatgcagcttgcagcaaggactaccccaaaagggcttcattgtctctcaatgcggttgacaacagaatattttaccctggatcacgaaaaaaggcagcttttgcaacaaagttataatgatcctgatctctaccattacgtagtcttctctgacaatgttttggcctcttcggttgttgttaactctacaatctcctcatcaaaggaaccggataaaatagtattccatgtggtgacagattcactcaattacccagcaatctcaatgtggtttttactaaacccaagtggcagagcttcaatccaaatcctaaacattgatgaaatgaatgtcctgccattgtaccatgctgaattgctgatgaagcaaaattcaagtgacccaagaatcatttcagcgctcaaccatgcacgcttctatctcccagatatcttcccaggtctaaacaagatcgtactcttcgatcatgatgtagtagtgcaaagggatctaactagactgtggagccttgatatgacggggaaagttgttggagctgtagagacttgtcttgaaggtgatccttcatatcgttcgatggactcattcattaatttctcagatgcatgggtttctcagaaatttgatcccaaggcttgcacttgggcattcgggatgaatctatttgatctcgaagaatggagaagacaggagttgacttctgtatacctgaaatacttcgacctgggagtaaaaggacatctgtggaaagcagggggattgccagtaggttggttgacttttttcgggcaaacgtttccgttggaaaagagatggaacgtgggtgggttaggtcacgaatcaggactcagggcaagcgacatcgaacaagcagcggttatacactacgacgggatcatgaaaccatggctggacatcggtatagacaagtacaagcgctactggaacatacatgtaccttaccatcaccctcacttacaacggtgcaacattcacgattga

SEQ ID NO: 20 - protein, 610 aa, Arabidopsis thaliana
MNQVRRWQRILILSLLLLSVLAPIVFVSNRLKSITSVDRGEFIEELSDITDKTEDELRLTAIEQDEEGLKEPKRILQDRDFNSVVLSNSSDKSNDTVQSNEGDQKNFLSEVDKGNNHKPKEEQAVSQKTTVSSNAEVKISARDIQLNHKTEFRPPSSKSEKNTRVQLERATDERVKEIRDKIIQAKAYLNLALPGNNSQIVKELRVRTKELERATGDTTKDKYLPKSSPNRLKAMEVALYKVSRAFHNCPAIATKLQAMTYKTEEQARAQKKQAAYLMQLAARTTPKGLHCLSMRLTTEYFTLDHEKRQLLQQSYNDPDLYHYVVFSDNVLASSVVVNSTISSSKEPDKIVFHVVTDSLNYPAISMWFLLNPSGRASIQILNIDEMNVLPLYHAELLMKQNSSDPRIISALNHARFYLPDIFPGLNKIVLFDHDVVVQRDLTRLWSLDMTGKVVGAVETCLEGDPSYRSMDSFINFSDAWVSQKFDPKACTWAFGMNLFDLEEWRRQELTSVYLKYFDLGVKGHLWKAGGLPVGWLTFFGQTFPLEKRWNVGGLGHESGLRASDIEQAAVIHYDGIMKPWLDIGIDKYKRYWNIHVPYHHPHLQRCNIHD

SEQ ID NO: 21 - DNA, 1770 bp, Arabidopsis thaliana
atgaaacaaattcgtcgatggcagaggattttgatcctcgctctgctatcgatatctgtattcgctccgcttattttcgtatcgaatcggcttaagagcatcactcccgttggtcgtagagaatttattgaagagttatccaaaattagattcacgacaaatgaccttagacttagcgctattgaacatgaggatggagaaggcttgaaggggccaaggctcattctcttcaaggatggggagtttaattcgtctgctgaaagtgatggtggtaatacttacaaaaacagggaagaacaagtgattgtttcacagaagatgacagttagctctgatgaaaagggtcaaattctaccaacagtcaaccaacttgctaataaaacggatttcaagccccctttatctaagggtgaaaagaacacaagggttcagcccgacagagcaacagatgtgaaaacgaaggagatcagagacaaaattattcaagctaaagcctacctgaatttcgctccacctggaagtaactctcaagttgtgaaggagttgagaggtcggctgaaagagctggaacggtctgttggtgatgcaacaaaggacaaggacttatcaaagggcgctctccgcagggtgaagcccatggaaaatgtgttatataaggctagtcgtgtctttaacaattgccctgccatcgctaccaaactccgtgccatgaattataacacagaagaacaagttcaggcgcagaaaaatcaagcagcgtatctaatgcagcttgcagcaaggaccaccccaaaagggcttcactgtctctcaatgcggctgacatcagaatacttttcactggatcctgaaaaaaggcagatgcctaaccagcaaaattattttgacgctaatttcaatcattatgttgtcttctctgacaatgttttggcttcttcagtcgttgttaactctacgatatcttcatcaaaggagccagaaagaatagtcttccatgtcgtgactgattcacttaattacccagcaatctcaatgtggtttctgctaaacattcaaagtaaagctactatccaaatcctaaacattgatgatatggatgtcctgcctagagattatgatcaattactgatgaagcaaaactctaatgacccaagattcatttctacactcaatcacgcacgcttctatctcccggatatattcccgggtttgaacaagatggtactcttggaccatgatgtagttgttcaaagagatttaagtagactgtggagcattgatatgaaaggaaaggtggttggagctgtagagacttgtcttgaaggtgaatcttcatttcgatcaatgagcacatttattaatttctcagacacatgggtcgctgggaaatttagtcctagagcttgcacatgggctttcgggatgaatctaattgatctcgaagaatggagaatacggaagttgacttctacatacataaaatacttcaacctgggaacaaagagaccattgtggaaagctgggagcttaccaataggttggttgactttctataggcaaacattagcattggacaagagatggcatgtgatggggttaggtcgcgaatcaggagtcaaagcggttgacatcgaacaagcggcagttatacactacgatggggtcatgaagccgtggttggacattggaaaagagaattacaaacgttactggaacatacacgtcccttaccatcacacctacttgcaacagtgcaatcttcaagcttga

SEQ ID NO: 22 - protein, 589 aa, Arabidopsis thaliana
MKQIRRWQRILILALLSISVFAPLIFVSNRLKSITPVGRREFIEELSKIRFTTNDLRLSAIEHEDGEGLKGPRLILFKDGEFNSSAESDGGNTYKNREEQVIVSQKMTVSSDEKGQILPTVNQLANKTDFKPPLSKGEKNTRVQPDRATDVKTKEIRDKIIQAKAYLNFAPPGSNSQVVKELRGRLKELERSVGDATKDKDLSKGALRRVKPMENVLYKASRVFNNCPAIATKLRAMNYNTEEQVQAQKNQAAYLMQLAARTTPKGLHCLSMRLTSEYFSLDPEKRQMPNQQNYFDANFNHYVVFSDNVLASSVVVNSTISSSKEPERIVFHVVTDSLNYPAISMWFLLNIQSKATIQILNIDDMDVLPRDYDQLLMKQNSNDPRFISTLNHARFYLPDIFPGLNKMVLLDHDVVVQRDLSRLWSIDMKGKVVGAVETCLEGESSFRSMSTFINFSDTWVAGKFSPRACTWAFGMNLIDLEEWRIRKLTSTYIKYFNLGTKRPLWKAGSLPIGWLTFYRQTLALDKRWHVMGLGRESGVKAVDIEQAAVIHYDGVMKPWLDIGKENYKRYWNIHVPYHHTYLQQCNLQA

SEQ ID NO: 23 - skipped

SEQ ID NO: 24 - protein, 605 aa, Populus trichocarpa
MKKFRRWQRIFLLSLLCLTVLAPILFVSVGRKELISDLSTLRYRRDSVQLNAIEQEEGEGLKGPKLVVYDEKELGSRISYSTSEENNDSKKYGNIGEIDRGSKRSQRGGNTSIPLERTNHESREENRQIPQETVTSRSEAKLQGQSNQATVRHDQNMRSPVRIFTDEKVKQMKDDLIRAKAYLSMTPPGSNSHLVKELRLRIKESERAVSAANKDSDLSRSALQKKRSLEVTLSKASRVFPDCSAMALKLRAMTYNAEEQVRAQKNQATYLVQLSGRTTPKGLHCLSMRLTAEYFALSPEERQLPNQQRVHDADLYHYAVFSDNVLACAVVVNSTVSSAMEPEKIVFHIVTDSLNLPTISMWFLLNPPGKATIQIQSLVDFKGLSANYNSTLKQLNSRDSRYTSALNHLRFYLPDVFPQLNKIVLFDHDVVVQKDLAGLWSLNMKGKVIGAVDTCREGEPSFRRMDKFINFSDPFVIKRFDAKACTWAFGMNLFDLQEWRRHKLTALYNKYLQLGHTRQLWKAGSLPLGWATFYNRTVILDRRWHKLGLGHEAGVGHDGVEQAAVLHYDGVMKPWLDIGIGKYKSYWSKHINYDHPYLQQCNIHE

SEQ ID NO: 25 - DNA, 1860 bp, Arabidopsis thaliana
atgaaaggcggaggcggtggtggaggaggtggtggcggaggaaaacgccggtggaaagttctggtgattggagttttggttcttgttattctttctatgcttgttcctcttgctttcttactcggtcttcacaatggctttcactctcctggatttgtcactgttcaaccggcttcttcatttgagagctttaccagaatcaatgctactaagcatacacagagagatgtatccgaacgggtcgatgaggttcttcaaaaaatcaatccagttcttcccaagaaaagcgacataaacgtgggttccagagatgtgaatgcaacaagcggcactgattctaaaaaaagaggattaccagtgtccccaactgttgttgccaatccaagccctgcaaataaaacaaaatcggaagcctcatatacaggtgttcagaggaaaatagtaagtggtgatgaaacttggagaacttgtgaagtgaaatatgggagctactgcctctggagggaggaaaataaggaaccaatgaaagatgccaaggtgaagcaaatgaaggaccagctgtttgtggctagagcatactatcccagtattgctaaaatgccttctcaaagcaagttgactcgggatatgaaacagaatatccaagagtttgagcgtattcttagtgaaagttctcaagatgctgaccttccaccacaggttgataaaaagttgcagaagatggaagctgtaattgcaaaggcaaagtcttttccagtcgactgtaacaatgttgacaagaaattgagacagatccttgatttgactgaggatgaagctagtttccacatgaaacagagtgtgttcctctaccagcttgcagtacagacaatgcctaagagtcttcattgcttgtcaatgcgactaactgtggaacatttcaagtcagattcacttgaggatcccattagtgagaaattttcagatccctcattacttcactttgttatcatctccgataatatactagcatcgtccgttgtgatcaactcaacggttgtacatgcaagggacagtaaaaactttgttttccatgtactgacagacgagcagaattactttgcaatgaaacaatggtttattaggaatccttgcaaacaatcaactgttcaagtattgaacattgaaaaactcgagctggacgattctgatatgaaactgtctttgtctgcggagttccgtgtttccttccccagtggtgaccttttggcgtctcaacagaatagaacacactacttatcccttttctctcaatctcactatcttcttcccaaattatttgacaaattggagaaggttgtgattctggatgatgacgttgtagtccagcgagacttatctcccctttgggaccttgatatggaagggaaagtgaatggcgctgttaagtcgtgcactgtgagattgggtcagctaaggagtctcaagagaggaaattttgataccaatgcttgtctctggatgtctggtttgaatgtcgttgatcttgctagatggagggcattgggtgtttcagaaacctatcaaaaatattataaagagatgagtagtggagatgagtcgagcgaagcaattgcattgcaggcaagcttgctcacatttcaagaccaagtatatgctcttgacgacaaatgggctctatcagggcttggttatgactactacatcaatgcacaagccataaaaaacgcagccatattgcactataacgggaacatgaagccgtggcttgagctgggaatcccaaattacaaaaactattggagaaggcatctgagtcgggaagatcggttcttgagtgactgtaacgtgaatccttga

SEQ ID NO: 26 - protein, 619 aa, Arabidopsis thaliana
MKGGGGGGGGGGGGKRRWKVLVIGVLVLVILSMLVPLAFLLGLHNGFHSPGFVTVQPASSFESFTRINATKHTQRDVSERVDEVLQKINPVLPKKSDINVGSRDVNATSGTDSKKRGLPVSPTVVANPSPANKTKSEASYTGVQRKIVSGDETWRTCEVKYGSYCLWREENKEPMKDAKVKQMKDQLFVARAYYPSIAKMPSQSKLTRDMKQNIQEFERILSESSQDADLPPQVDKKLQKMEAVIAKAKSFPVDCNNVDKKLRQILDLTEDEASFHMKQSVFLYQLAVQTMPKSLHCLSMRLTVEHFKSDSLEDPISEKFSDPSLLHFVIISDNILASSVVINSTVVHARDSKNFVFHVLTDEQNYFAMKQWFIRNPCKQSTVQVLNIEKLELDDSDMKLSLSAEFRVSFPSGDLLASQQNRTHYLSLFSQSHYLLPKLFDKLEKVVILDDDVVVQRDLSPLWDLDMEGKVNGAVKSCTVRLGQLRSLKRGNFDTNACLWMSGLNVVDLARWRALGVSETYQKYYKEMSSGDESSEAIALQASLLTFQDQVYALDDKWALSGLGYDYYINAQAIKNAAILHYNGNMKPWLELGIPNYKNYWRRHLSREDRFLSDCNVNP

SEQ ID NO: 27 - skipped

SEQ ID NO: 28 - protein, 590 aa, Populus trichocarpa
MKGYHNNHNQGKRRWRCLVIGVLFLVLLSMLVPLVFLLGLYHNGFHSTGAPAVPPAVPQPPLRRNVRMHTSECFPENVIHFVMLLKPLEFVFNMLWQNAVTTGTDEITKHKRSAFEESEKCELRFGGYCHWCDEHRESMKDFMVNKLKDQLFVARAYYPTIAKLLSQEKLTNEMRQNIQELERILSESSTDADLPPQIQKNLQKMENVIAKAKTFPVDCNNVDKKLRQILDLTEEETNFHMKQSAFLYQLAVQTMPKGLHCLSMRLLVEYFKSSVHDKELPLSERYSNPSLQHYVILSTNVLAASVVINSTAVHARESGNLVFHVLTDGLNYFAMKLWFLRNTYKEAAVQVLNVENVTLKYHDKEALKSMSLPLEYRVSFHTVNNPPATHLRTEYVSVFSHTHYLIPSIFEKLKRVVVLDDDVVVQRDLSDLWNIDMGGKVNGALQLCSVQLGQLRNFLGKGSFDENSCAWMSGLNVIDLVRWRELDLTKTYWKLGQEVSKGTGSAEAVALSTSLLTFQDLVYPLDGVWALSGLGHDYGIDVQAIKKAAVLHFNGQMKPWLELGIPKYKQYWKRFLNRDDLFLGECNVNP

SEQ ID NO: 29 - skipped

SEQ ID NO: 30 - protein, 620 aa, Populus trichocarpa
MKGYHNNHNQGKRRWRCLVIGVLFLVLLSMLVPLVFLLGLYHNGFHSTGNSLQQHLSLFHPPPPSQIQLPFHFFCCFLLSNLTDTYTLYFLLNTRQPDLFFFLSHQMNSITKLCHSSSSAGHLSDRQTSSASAVYEITKHKRNAVEESEKCELRFGGYCHWRDEHRENMKDFMVKKLKDQLFVARAYYPSIAKLPSQEKLTHELKQNIQELERILSESSTDADLPPQIQKKLQKMENVISKAKTFPVDCNNVDKKLRQILDLTEEETNFHMKQSAFLYQLAVQTMPKGLHCLSMRLIVEYFKSSAHDKEFPLSERYSDPSLQHYVVFSTNVLAASVVINSTAVHARESGNLVFHVLTDGLNYYAMKLWFLRNTYKEAAVQVLNIENVTLKYYDKEVLKSMSLPVEYRVSFQTVTNPPASHLRTEYVSVFSHTHYLLPYIFEKLKRVVVLDDDVVVQRDLSDLWNLNMGRKVNGALQLCSVQLGQLRSYLGKSIFDKTSCAWMSGLNVIDLVRWRELDLTKTYWKLGQEVSKGTESDESVALSTSLLTFQDLVYPLDGAWALSGLGHDYGIDVQAIKKASVLHFNGQMKPWLEVGIPKYKHYWKRFLNRHDQLLVECNVNP

SEQ ID NO: 31 - DNA, 1680 bp, Arabidopsis thaliana
atggctaatcaccaccgacttttacgcggcggcggatctccggccataatcggtggcagaatcacactcacagctttcgcttccactatcgcactcttcctcttcactctctccttcttcttcgcttcagattctaacgattctcctgatctccttcttcccggtgttgagtactctaatggagtcggatctagaagatccatgttggatatcaaatcggatccgcttaagccacggttgattcagatccggaaacaagctgatgatcatcggtcattagcattagcttatgcttcttacgcgagaaagcttaagctcgagaattcgaaactcgtcaggatcttcgctgatctttcgaggaattacacggatctgattaacaaaccgacgtatcgagctttgtatgattctgatggagcctcgattgaagaatctgtgcttaggcaatttgagaaagaagttaaggaacggattaaaatgactcgtcaagtgattgctgaagctaaagagtcttttgataatcagttgaagattcagaagctgaaagatacgattttcgctgttaacgaacagttaactaatgctaagaagcaaggtgcgttttcgagtttgatcgctgcgaaatcgattccgaaaggattgcattgtcttgctatgaggctgatggaagagaggattgctcaccctgagaagtatactgatgaagggaaagatagaccgcgggagctcgaggatccgaatctttaccattacgctatattttcggataatgtgattgcggcttcggtggttgtgaactctgctgtgaagaatgctaaggagccgtggaagcatgtttttcacgttgtgactgataagatgaatcttggagctatgcaggttatgtttaaactgaaggagtataaaggagctcatgtagaagttaaagctgttgaggattatacgtttttgaactcttcgtatgtgcctgtgttgaagcagttagaatctgcgaatcttcagaagttttatttcgagaataagctcgagaatgcgacgaaagataccacgaatatgaagttcaggaaccccaagtatttatctatattgaatcacttgaggttttatttacccgagatgtacccgaaactacataggatactgtttttggacgatgatgtggttgtgcagaaggatttaacgggtctgtgggagattgatatggatgggaaagtgaatggagctgtagagacttgttttgggtcgtttcatcggtacgctcaatacatgaatttctcacatcctttgatcaaagagaagtttaatcccaaagcatgtgcgtgggcgtatggaatgaacttctttgatcttgatgcttggagaagagagaagtgcacagaagaatatcactactggcaaaatctgaacgagaacagggctctatggaaactggggacgttaccaccgggactgatcaccttttactcaaccacaaagccgctggacaaatcatggcatgtgcttgggctgggttacaatccgagcattagcatggatgagatccgcaacgctgcagtggtacacttcaacggtaacatgaagccatggcttgacatagctatgaaccagtttcgaccactttggaccaaacacgtcgactatgacctcgagtttgttcaggcttgcaattttggcctctga

SEQ ID NO: 32 - protein, 559 aa, Arabidopsis thaliana
MANHHRLLRGGGSPAIIGGRITLTAFASTIALFLFTLSFFFASDSNDSPDLLLPGVEYSNGVGSRRSMLDIKSDPLKPRLIQIRKQADDHRSLALAYASYARKLKLENSKLVRIFADLSRNYTDLINKPTYRALYDSDGASIEESVLRQFEKEVKERIKMTRQVIAEAKESFDNQLKIQKLKDTIFAVNEQLTNAKKQGAFSSLIAAKSIPKGLHCLAMRLMEERIAHPEKYTDEGKDRPRELEDPNLYHYAIFSDNVIAASVVVNSAVKNAKEPWKHVFHVVTDKMNLGAMQVMFKLKEYKGAHVEVKAVEDYTFLNSSYVPVLKQLESANLQKFYFENKLENATKDTTNMKFRNPKYLSILNHLRFYLPEMYPKLHRILFLDDDVVVQKDLTGLWEIDMDGKVNGAVETCFGSFHRYAQYMNFSHPLIKEKFNPKACAWAYGMNFFDLDAWRREKCTEEYHYWQNLNENRALWKLGTLPPGLITFYSTTKPLDKSWHVLGLGYNPSISMDEIRNAAVVHFNGNMKPWLDIAMNQFRPLWTKHVDYDLEFVQACNFGL

SEQ ID NO: 33 - skipped

SEQ ID NO: 34 - protein, 554 aa, Populus trichocarpa
MATHRSSRSGVGVSFRVLGSAVSLAVFLCLTVSLLFTAHSHSTTDTHGFSNVGYGLGSGRRSVLAMKSDPLKSRLDQIRKQADDHRSLAHAYASYARKLKLENSKLVRVFADLSRNYTDLINKPSYRALSESDSLSIDEATLRLFEKEVKERIKVTRQVIAEAKESFDNQLKIQKLKDTIFAVNEQLTKAKKQGAFSSLIAAKSIPKSLHCLAMRLMEERIAHPEKYNDEGKPPLPELEDPKLYHYAIFSDNVIAASVVVNSAVKNAKEPWKHVFHVVTDKMNLGAMQVMFKLKDYNGAHIEVKAVEDYKFLNSSYVPVLKQLESANLQKFYFENKLENATKDTTNMKFRNPKYLSILNHLRFYLPEMYPKLHRILFLDDDIVVQKDLTGLWKIDMDGKVNGAVETCFGSFHRYAQYMNFSHPLIKEKFNPKACAWAYGMNFFDLDAWRREKCTEEYHYWQNLNENRTLWKLGTLPPGLITFYSTTKPLDKSWHVLGLGYNPSISMDEIQSAAVVHFNGNMKPWLDIAMTQFKPLWTKHVDYELEFVQACNFGL

SEQ ID NO: 35 - DNA, 1686 bp, Arabidopsis thaliana
atggcggtggccttccgtggaggccggggaggcgtcggatccggccaatctaccggacttcgtagtttcttctcctaccggatctttatctccgctttgttctcttttctcttcctcgccactttctccgtcgttcttaactcctctcgtcatcagcctcatcaggatcatacattgccgagtatgggcaacgcatatatgcagaggacgtttttggctttgcaatcggatccattgaaaactaggttggatctgatacacaagcaagccattgatcatttgacactggtgaatgcgtatgctgcttacgctaggaagctaaagcttgatgcttctaagcagcttaagctcttcgaagatttggctatcaacttctcggatttgcagtcgaaacctggtttgaaatctgctgtgtctgataatggtaatgctcttgaggaggattcgtttaggcagcttgagaaagaagtgaaggataaggtgaagacagcgaggatgatgatcgttgagtctaaagagagttatgatacacagcttaaaatccagaagttgaaagatacaatctttgctgtccaagaacagttgacaaaggctaagaaaaacggtgcggttgctagcttgatttcagccaagtcggttcctaaaagtcttcattgtttggccatgaggcttgtaggagagaggatctctaatcctgagaagtacaaggatgctccacctgacccagccgcagaggatccaactctttaccactatgcgattttctctgataatgtcattgctgtgtctgttgtggtgagatcggttgtgatgaacgctgaggagccatggaagcatgtcttccatgtggtgacagatcggatgaatctcgcagccatgaaggtgtggtttaagatgcgtcctttggaccgtggtgcccatgttgagattaaatccgtggaggatttcaagttcttaaactcttcctatgcgccggtcttgaggcagcttgagtctgccaagttgcagaagttttactttgagaatcaagctgagaacgcaactaaagattcacataacctcaagttcaagaaccccaagtatctctcgatgttgaaccatctcagattttacttaccagagatgtatccgaagctgaataagattttgttcttggacgatgatgttgtggtgcagaaagacgtgactggtttatggaaaatcaacttggatggcaaggtgaatggagccgttgagacatgttttggttcttttcatcgatatggtcaatacttaaacttctctcatcctttgatcaaagagaactttaaccccagtgcctgtgcttgggcctttggaatgaacatattcgatctcaatgcctggagacgcgagaagtgcaccgatcaataccattactggcagaacctgaatgaagacagaactctctggaaattgggaactctacctccgggattgatcacattctattcaaagacgaaatcattggacaaatcatggcatgtacttgggttaggctataacccgggagtgagcatggacgaaatcagaaatgcaggagtgattcattacaatggaaacatgaaaccgtggctagacattgcgatgaaccaatacaagtctctctggactaaatatgttgataacgaaatggagtttgtgcagatgtgcaattttggtctctaa

SEQ ID NO: 36 - protein, 561 aa, Arabidopsis thaliana
MAVAFRGGRGGVGSGQSTGLRSFFSYRIFISALFSFLFLATFSVVLNSSRHQPHQDHTLPSMGNAYMQRTFLALQSDPLKTRLDLIHKQAIDHLTLVNAYAAYARKLKLDASKQLKLFEDLAINFSDLQSKPGLKSAVSDNGNALEEDSFRQLEKEVKDKVKTARMMIVESKESYDTQLKIQKLKDTIFAVQEQLTKAKKNGAVASLISAKSVPKSLHCLAMRLVGERISNPEKYKDAPPDPAAEDPTLYHYAIFSDNVIAVSVVVRSVVMNAEEPWKHVFHVVTDRMNLAAMKVWFKMRPLDRGAHVEIKSVEDFKFLNSSYAPVLRQLESAKLQKFYFENQAENATKDSHNLKFKNPKYLSMLNHLRFYLPEMYPKLNKILFLDDDVVVQKDVTGLWKINLDGKVNGAVETCFGSFHRYGQYLNFSHPLIKENFNPSACAWAFGMNIFDLNAWRREKCTDQYHYWQNLNEDRTLWKLGTLPPGLITFYSKTKSLDKSWHVLGLGYNPGVSMDEIRNAGVIHYNGNMKPWLDIAMNQYKSLWTKYVDNEMEFVQMCNFGL

SEQ ID NO: 37 - skipped

SEQ ID NO: 38 - protein, 504 aa, Populus trichocarpa
SLPSSGNAYVQRTFLAIKSDPLKTRLDLIYKQANDHMTLVNAYAAYARKLKLDISRQLRMFDELDKNLTDLPLKPSYKSSLFEPGSDVDEDVLRQFEKEVKEKVKVARLMIAEAKESYDNQIKIQKLKDTIFAVNELLIKAKKNGAFASLISAKSVPKSLHCLAMRLVGERIAHPEKYKEEGYKAEFEDPSLYHYAIFSDNVIAVSVVIRSVVKNAEEPWKHVFHVVTDKMNVAAMKVWFRMRPVEGGAHVEINAVEDFSFLNSSYVPVLKQLESAKMQKFYFDNQAENATKDGSNMKFRNPKYMSMLNHLRFYLPEMYPKLHKILFLDDDVVVQKDLTGLWKVDLDGKVNGAVETCFGSFHRYAQYLNFSHPLIKERFNPKACAWAFGMNIFDLDAWRREKCTEHYHYWQSLNEDRTLWKLGTLPPGLITFYSTTKSLDKSWHVLGLGYNPSISMDEISNAAVIHYNGNMKPWLDIAMNQYKNLWTKYVDNDMEFVQMCNFGL

SEQ ID NO: 39 - DNA, 1611 bp, Arabidopsis thaliana
atgagaaggagaggaggggatagtttccggagagctggacggaggaagatctcgaatgtggtatggtgggttctctctggtattgccctcctgctcttctttctcattctctccaaagctggtcatattgaacctagaccctctattcctaagcgacgttaccgtaatgacaaatttgtagagggtatgaatatgactgaggaaatgttgagtcctacttccgttgctcgtcaagttaatgatcagattgctcttgctaaagcttttgttgtcattgctaaagaaagtaagaatcttcagtttgcttgggacttaagtgctcagatccgtaactctcagttgcttttatcgagtgctgctactaggagaagtcccttgactgtcttggaatctgagtctactattcgtgacatggctgttttgttatatcaagctcagcagcttcactatgatagtgctactatgattatgaggcttaaggcctcgattcaggctcttgaagaacaaatgagttccgttagcgagaagagttccaagtatggacagattgctgctgaggaagtgcctaagagtctttactgtcttggtgttcgtctcactaccgaatggtttcagaatttagacttacagagaactcttaaggaaaggagtcgtgttgattcgaaactcacggataacagtctctaccatttctgtgtgttttccgataacattattgctacttctgttgtggttaattctactgctctcaattccaaggcccctgagaaagttgtgtttcatcttgtgactaatgagatcaactatgctgcaatgaaggcttggttcgccattaatatggacaacctcagaggagtcactgtggaggttcagaagttcgaggatttctcatggctgaatgcttcctatgttccggtcctcaagcagctgcaagactctgatacgcaaagctattatttctctggacacaacgatgatgggcgcactccaatcaaattcaggaaccccaagtatctttccatgctcaaccatcttaggttctacatccctgaagtgtttcctgcgctgaagaaggtggtctttcttgatgatgatgttgtagttcagaaggatctttcatctctcttttcgatcgatttaaacaaaaatgtgaacggggctgttgagacctgcatggagaccttccaccgctaccacaagtacttgaactattctcatcctctcatacgctcccactttgatccagatgcgtgtgggtgggcgtttggaatgaacgtctttgatttagttgagtggaggaagagaaatgtgaccggcatataccactactggcaagaaaaaaacgtggaccggaccttatggaaactgggaacactacctccaggacttctgacattttacgggttaacagaggcactagaggcgtcctggcatatcctgggattgggatacacgaatgtggatgctcgtgtgatagagaaaggagctgttcttcacttcaatgggaacttaaagccatggttgaagatcgggatagagaagtacaaacctttgtgggagagatacgttgattacacttctccttttatgcaacaatgcaattttcattga

SEQ ID NO: 40 - protein, 536 aa, Arabidopsis thaliana
MRRRGGDSFRRAGRRKISNVVWWVLSGIALLLFFLILSKAGHIEPRPSIPKRRYRNDKFVEGMNMTEEMLSPTSVARQVNDQIALAKAFVVIAKESKNLQFAWDLSAQIRNSQLLLSSAATRRSPLTVLESESTIRDMAVLLYQAQQLHYDSATMIMRLKASIQALEEQMSSVSEKSSKYGQIAAEEVPKSLYCLGVRLTTEWFQNLDLQRTLKERSRVDSKLTDNSLYHFCVFSDNIIATSVVVNSTALNSKAPEKVVFHLVTNEINYAAMKAWFAINMDNLRGVTVEVQKFEDFSWLNASYVPVLKQLQDSDTQSYYFSGHNDDGRTPIKFRNPKYLSMLNHLRFYIPEVFPALKKVVFLDDDVVVQKDLSSLFSIDLNKNVNGAVETCMETFHRYHKYLNYSHPLIRSHFDPDACGWAFGMNVFDLVEWRKRNVTGIYHYWQEKNVDRTLWKLGTLPPGLLTFYGLTEALEASWHILGLGYTNVDARVIEKGAVLHFNGNLKPWLKIGIEKYKPLWERYVDYTSPFMQQCNFH

SEQ ID NO: 41 - skipped

SEQ ID NO: 42 - protein, 534 aa, Populus trichocarpa
MRRRPVDFRRPVRRRVSNVVVWSLCGIVVLLFIVIFSKESRIESRPTSSIKDYTKHVKNIEGLNITDEMLSPNSVTRQLSDQISLAKAFVVIAKESNNIQFAWELSAQIRNSQVLLSSVATRRAPLTTRESETAIRDMALLLVQAQQLHYDSATMIMRLKTKIQTLDEQMAAVSEKSSKYGQIAAEEIPKGLYCLGIRLTTEWFGNSNLHRRMNERMHIETKLRDNSLYHFCVFSDNILATSVVVNSTTLNSKNPDMVVFHLVTDEINYAAMKAWFSMNTFRGVTIEVQNFEDFKWLNASYVPVLKQLQDSETQSYYFSGHNNDGQTPIKFRNPKYLSMLNHLRFYIPEVFPALEKVVFLDDDVVVQKDLSGLFSIDLNSNVNGAVETCMETFHRYHKYLNYSHPLIREHFDPDACGWAFGMNVFDLVEWRKRNVTEIYHYWQEKNVDRTLWKLGTLPPGLLTFYGLTEPLDPSWHVLGLGYTNVDPHLIEKGAVLHFNGNSKPWLKIGMEKYKSLWEKYVDYSHPLLQQCNFH

SEQ ID NO: 43 - skipped

SEQ ID NO: 44 - protein, 534 aa, Populus trichocarpa
MRRRPVDFRRPVRRRISSVVWWTLCGISVLLFIVIFSKESRIESRSTSFNKYYTKYEKNIEGLNITDEMLSPNSITRQLSDQISLAKAFVVIAKESNNLQFAWELSAQIRNSQVLLSSAATRRAPLTTRESETAIRDMALLLFQAQQLHYDSATMIMRLKAKIQVLDEQMGIVNEKSSKYGQIAAEEIPKGLYCIGIRLTTEWFGNPNLQRKKNERMQIQTKLRDSNLYHFCVFSDNILATSVVVNSTALNSKNPDMVVFHLVTDEINYIAMKAWFAMNTFRGVTVEVQKFEDFKWLNASYVPVLKQLQDSETQSYYFSGHNDDGRTPIKFRNPKYLSMLNHLRFYIPEVFPALKKVVFLDDDVVVQKDLSGLFSVDLNSNVNGAVETCMETFHRYHKYLNYSHPLIREHFDPDACGWAFGMNVFDLVEWRKRNVTEIYHYWQEKNVDRTLWKLGTLPPGLLTFYGLTEPLDPSWHVLGLGYTNVDPHLIEKGAVLHFNGNSKPWLKIGMEKYKPLWEKHVDYSHPLLQQCNFH

SEQ ID NO: 45 - DNA, 1614 bp, Arabidopsis thaliana
atgaggcggtggccggtggatcaccggcggcgaggtagaaggagattgtcgagttggatatggtttctccttggttctttctctgtcgctggtttagttctcttcatcgttcagcattatcaccatcaacaagatccatcccagcttttacttgagagagacacgagaaccgaaatggtatctcctccccatttaaacttcacggaagaggtcacaagtgcttcctccttctctaggcagttagcagagcaaatgacacttgccaaagcttatgtgtttatagctaaagagcataataatcttcatttagcttgggaattgagttctaagatcagaagttgtcagcttttgctttccaaagcagctatgagaggacaacctatttcgtttgatgaggctaaaccgattattactggtctatcagctcttatctacaaggctcaagatgcacattatgatattgccaccactatgatgaccatgaaatctcacatccaagcacttgaagagcgtgcaaatgcagctactgttcagaccacaatatttgggcaattggttgctgaggcattaccaaagagcctccactgtttgacgataaagctcacatctgattgggtaacagagccatctcgccatgaactggcagatgagaacagaaactcacctagacttgtcgacaacaacctctaccacttctgcatcttctcggacaacgtgattgccacctcggttgttgttaattcaactgtctcgaatgctgatcatccaaagcagcttgttttccacatagtgacgaatcgagtgagctacaaagctatgcaggcctggtttctaagtaatgacttcaagggctcagcaatagagatcaggagcgtagaggagttttcttggttgaatgcttcatattctcctgttgttaagcaactgctggacacagatgcaagagcttactatttcggggaacagacaagtcaagatacgatttccgagccaaaagtgaggaacccaaagtacttgtcattactgaaccatctcagattctacattccggagatctatccacagctagagaagattgttttcctagacgatgatgttgttgttcagaaagatttgactccactcttctccttggatctgcatggaaacgtcaatggagctgtggaaacatgtcttgaagcctttcaccgatattacaagtatctaaatttctcgaacccactcatcagctcaaagttcgacccacaagcatgtggatgggcttttggtatgaacgtttttgatctgatcgcttggaggaatgcaaacgtgactgctcggtaccattactggcaagatcagaacagagaacgaacgctttggaaactcgggacactccctccaggtctactatctttctatggtctcacagagccactggacagaagatggcatgtcttgggtttaggttacgatgtgaacatcgataaccgtctgatcgaaacagcagctgtgattcactataatggtaacatgaagccttggctaaagctggctattggtaggtataaacctttctggttaaagtttttgaactcgagccatccttatttacaagattgtgtcacagcttaa

SEQ ID NO: 46 - protein, 537 aa, Arabidopsis thaliana
MRRWPVDHRRRGRRRLSSWIWFLLGSFSVAGLVLFIVQHYHHQQDPSQLLLERDTRTEMVSPPHLNFTEEVTSASSFSRQLAEQMTLAKAYVFIAKEHNNLHLAWELSSKIRSCQLLLSKAAMRGQPISFDEAKPIITGLSALIYKAQDAHYDIATTMMTMKSHIQALEERANAATVQTTIFGQLVAEALPKSLHCLTIKLTSDWVTEPSRHELADENRNSPRLVDNNLYHFCIFSDNVIATSVVVNSTVSNADHPKQLVFHIVTNRVSYKAMQAWFLSNDFKGSAIEIRSVEEFSWLNASYSPVVKQLLDTDARAYYFGEQTSQDTISEPKVRNPKYLSLLNHLRFYIPEIYPQLEKIVFLDDDVVVQKDLTPLFSLDLHGNVNGAVETCLEAFHRYYKYLNFSNPLISSKFDPQACGWAFGMNVFDLIAWRNANVTARYHYWQDQNRERTLWKLGTLPPGLLSFYGLTEPLDRRWHVLGLGYDVNIDNRLIETAAVIHYNGNMKPWLKLAIGRYKPFWLKFLNSSHPYLQDCVTA

SEQ ID NO: 47 - skipped

SEQ ID NO: 48 - protein, 531 aa, Populus trichocarpa
MRRRPAEYRRPVRRRLSQWIWALIGMFLIAGLVLFVFLHNHHEDQVNQPIMGEHAIKRGGFNFTKEILNASSFSRQLAEQMTLAKAYVIIAKEHNNLHLAWELSKKIRSCQLLLSKAAMRGEPITVEEAEPIISSLSYLIFKAQDAHYDIATTMMTMKSHIQALEERTNAATVQSTLFGQLVAEVLPKSLHCLKVKLINDWLKQLPLQNHAEEKRNSPRVVDNNLYHFCIFSDNILATSVVVNSTVCNADHPKQLVFHIVTNGISYGSMQAWFLTNDFKGATVEVQNIEEFSWLNASYAPVIKQIIHQDSRAYYFGADQDMKVEPKLRNPKYLSLLNHLRFYIPEIYPLLEKIVFLDDDVVVQKDLTRLFSLDLHGNVNGAVETCLETFHRYYKYINFSNPIISSKFDPQACGWAFGMNIFDLIAWRKENVTAQYHYWQEQNADQTLWKLGTLPPALLAFYGLTEPLDRRWHVLGLGYDMNIDDRLIDSAAVIHFNGNMKPWLKLAISRYKPLWERYVNQSHPYYQDCVTS

SEQ ID NO: 49 - skipped

SEQ ID NO: 50 - protein, 489 aa, Populus trichocarpa
MFLVQGENATKEPLNHEGLNFTKEILSASSFSRQLAEQMTLAKAYVIIAKEHNNLHLAWELSNKIRSCQLLLSKAAKRGESITVEEAEPIISSLSYLIFKAQDAHYDISTTMMTMKSHIQALEERTNAATVQSTLFGQLVAEALPKSLHCLKVKLTNDWLKQLPLQNHVEEKRNSPRVIDNNLNHFCIFSDNVLATSVVVNSTISNADHPKQLVFHIVTNGISYGSMQVWFLTNDFKGATVEVQNIEEFTWLNASYAPVIKRLLDQDSRAYYFGAYQDMKVEPKLRNPKHMSLLNHLRFYIPEVYPLLEKVVFLDDDVVVQKDLTRLFSLDLHGNVNGAVETCLEAFHRYYKYINFSNPVISSKFDPQACGWAFGMNVFDLIAWRKENVTARYHYWQEQNGDQMLWKLGTLPPALLAFYGLTETLDRRWHVLGLGYDMNIDDRLIDSAAVIHFNGNMKPWLKLAIGRYKPLWERYINQSHPYYQDCVIS

SEQ ID NO: 51 - DNA, 1608 bp, Arabidopsis thaliana
atgcagttacatatatctccgagcttgagacatgtgactgtggtcacagggaaaggattgagagagttcataaaagttaaggttggttctagaagattctcttatcaaatggtgttttactctctactcttcttcacttttcttctccgattcgtctttgttctctccaccgttgatactatcgacggcgatccctctccttgctcctctcttgcttgcttggggaaaagactaaagccaaagcttttaggaagaagggttgattctggtaatgttccagaagctatgtaccaagttttagaacagcctttaagcgaacaagaactcaaaggaagatcagatatacctcaaacacttcaagatttcatgtctgaagtcaaaagaagcaaatcagacgcaagagaatttgctcaaaagctaaaagaaatggtgacattgatggaacagagaacaagaacggctaagattcaagagtatttatatcgacatgtcgcatcaagcagcataccgaaacaacttcactgtttagctcttaaactagccaacgaacactcgataaacgcagcggcgcgtctccagcttccagaagctgagcttgtccctatgttggtagacaacaactactttcactttgtcttggcttcagacaatattcttgcagcttcggttgtggctaagtcgttggttcaaaatgctttaagacctcataagatcgttcttcacatcataacggataggaaaacttatttcccaatgcaagcttggttctcattgcatcctctgtctccagcaataattgaggtcaaggctttgcatcatttcgattggttatcgaaaggtaaagtacccgttttggaagctatggagaaagatcagagagtgaggtctcaattcagaggtggatcatcggttattgtggctaataacaaagagaacccggttgttgttgctgctaagttacaagctctcagccctaaatacaactccttgatgaatcacatccgtattcatctaccagagttgtttccaagcttaaacaaggttgtgtttctagacgatgacattgtgatccaaactgatctttcacctctttgggacattgacatgaatggaaaagtaaatggagcagtggaaacatgtagaggagaagacaagtttgtgatgtcaaagaagttcaagagttacctcaacttctcgaatccgacaattgccaaaaacttcaatccagaggaatgtgcatgggcttatggaatgaatgttttcgacctagcggcttggaggaggactaacataagctccacttactatcattggcttgacgagaacttaaaatcagacctgagtttgtggcagctgggaactttgcctcctgggctgattgctttccacggtcatgtccaaaccatagatccgttctggcatatgcttggtctcggataccaagagaccacgagctatgccgatgctgaaagtgccgctgttgttcatttcaatggaagagctaagccttggctggatatagcatttcctcatctacgtcctctctgggctaagtatcttgattcttctgacagatttatcaagagctgtcacattagagcatcatga

SEQ ID NO: 52 - protein, 535 aa, Arabidopsis thaliana
MQLHISPSLRHVTVVTGKGLREFIKVKVGSRRFSYQMVFYSLLFFTFLLRFVFVLSTVDTIDGDPSPCSSLACLGKRLKPKLLGRRVDSGNVPEAMYQVLEQPLSEQELKGRSDIPQTLQDFMSEVKRSKSDAREFAQKLKEMVTLMEQRTRTAKIQEYLYRHVASSSIPKQLHCLALKLANEHSINAAARLQLPEAELVPMLVDNNYFHFVLASDNILAASVVAKSLVQNALRPHKIVLHIITDRKTYFPMQAWFSLHPLSPAIIEVKALHHFDWLSKGKVPVLEAMEKDQRVRSQFRGGSSVIVANNKENPVVVAAKLQALSPKYNSLMNHIRIHLPELFPSLNKVVFLDDDIVIQTDLSPLWDIDMNGKVNGAVETCRGEDKFVMSKKFKSYLNFSNPTIAKNFNPEECAWAYGMNVFDLAAWRRTNISSTYYHWLDENLKSDLSLWQLGTLPPGLIAFHGHVQTIDPFWHMLGLGYQETTSYADAESAAVVHFNGRAKPWLDIAFPHLRPLWAKYLDSSDRFIKSCHIRAS

SEQ ID NO: 53 - skipped

SEQ ID NO: 54 - protein, 532 aa, Populus trichocarpa
MQLHISPSLRHVTVLPGNGVREFIKVKVRARRVSYRMLFYSLLFFTFLLRFVFLLSTADTIDAETKCSTLGCLGKRLGPRILGRRLDSAVPEVMYQVLEQPLDNDELKGRDDIPQTLEEFMDEVKNSIFDAKAFALKLREMVTLLEQRTRNAKIQEYLYRHVASSSIPKQLLCLALRLAHEHSTNAAARRQLPLPELVPALVDNSYFHFVLASDNVLAASVVANSLFQNALRPEKFVLHIITDRKTYSPMQAWFSLHPLSPAIIEVKALHHFDWFAKGKVPVLEAMEKDLRVRSRFRGGSSAIVESNTDKPHIIAAKLQTLGPKYNSVMNHIRIHLPELFPSLNKVVFLDDDIVVQTDLSPLWDIDMNGKVNGAVETCRGQDKFVMSKRLKNYLNFSHPLIAKNFNPNECAWAYGMNIFDLEAWRKTNISITYHHWVEENLKSGLSLWQLGTLPPGLIAFHGHVHVIDPFWHMLGLGYQENTSLADAETAGVIHFNGRAKPWLDIAFPQLRPLWAKYINSSDKFITGCHIRT

SEQ ID NO: 55 - skipped

SEQ ID NO: 56 - protein, 533 aa, Populus trichocarpa
MQLHISPSLRHVTVFPGKGVREFIKVRVGARRVSYRMLFYSLLFFTFLLRFVFVLSTVDSIDGETKCSTLGCLGKRLGPRILGRRLDSAVPEVMFQVLEQPLGNDELKGRSDIPQTLEEFMDEVKNTRLDAKTFALKLREMVTLLEQRTRNAKIQEYLYRHVASSSIPKQLHCLALRLASEHSTNAAARLQLPLPELVPALVDNTYFHFVLASDNVLAAAVVANSLVQNALRPQKFVLHIITDRKTYSPMQAWFSLHPLAPAIIEVKALHHFDWFAKGKVPVMEAMEKDQRVRSQFRGGSSAIVANNTEKPHIIAAKLQTLSPKYNSVMNHIRIHLPELFPSLNKVVFLDDDIVVQSDLSPLWDIDMNGKVNGAVETCRGEDKFVMSKKLKSYLNFSHPLISENFKPNECAWAYGMNIFDLEAWRKTNISTTYHHWVEENLKSDLSLWQLGTLPPGLIAFHGHVHVIDPFWHMLGLGYQENTSLADAETAGVIHFNGRAKPWLDIAFPQLRPLWAKYINFSDKFIKGCHIRPS

SEQ ID NO: 57 - DNA, 1602 bp, Arabidopsis thaliana
atgcagcttcacatatcgcctagcatgagaagcattacgatatcgagcagcaatgagtttattgatttgatgaagatcaaagtcgcagctcgtcacatctcttaccgaactctcttccacactatcttaatcctcgctttcttgttaccttttgttttcatcctaaccgctgttgttacccttgaaggtgtcaacaagtgctcctcttttgattgtttcgggaggcggctaggaccacgtcttcttggtaggatagatgattcagagcagagactagttagagatttttacaaaattctaaatgaagtaagcactcaagaaattccagatggtttaaagcttccagagtcttttagtcaactggtttcggatatgaagaacaaccactatgatgctaaaacatttgccctcgtatttcgagctatggtagagaagtttgaaagggatttaagggaatccaaatttgcagaactcatgaacaagcactttgctgcaagttcaattccaaaaggaattcactgtctctctttaagactaaccgatgaatattcctccaatgctcatgcccggagacagcttccttccccggagcttctccctgttctctcagacaatgcttaccaccattttgttctagctacagataatatcttagctgcatcggttgtggtctcatctgctgttcaatcatcttcaaaacccgagaaaattgtcttccatgttatcacagacaagaaaacctatgcgggtatgcattcttggtttgcactcaattctgttgctcctgcgattgttgaagtgaaaagcgttcatcagtttgattggttaacaagagagaatgttccagttcttgaagctgtggaaagccataacagtatcagaaattattaccatgggaatcatattgctggtgcaaacctcagcgaaacaacccctcgaacatttgcttcgaaactgcagtcaagaagtcccaaatacatatctttgctcaaccatcttagaatatatctaccagagctttttccgaacttagacaaggtagtgttcttagatgatgatatagtgatacagaaagatttatctccgctttgggatattgaccttaacgggaaggttaatggagctgtggagacttgtcgaggagaagacgtatgggttatgtcaaagcgtcttaggaactacttcaatttttctcacccgctcatcgcaaagcatttagatcccgaagaatgtgcttgggcttatggaatgaatatctttgatctacggacttggaggaagacaaatatcagagaaacgtatcattcttggcttaaagagaatctgaagtcgaatctaacaatgtggaaacttggaacattgcctcctgctctaatagcatttaaaggtcatgttcagccaatagattcctcttggcatatgcttggattaggttatcagagcaagaccaacttagaaaatgcgaagaaagctgcagtgattcattacaatggccaatcaaagccgtggcttgagataggtttcgagcatctcagaccattctggacaaaatatgttaactactccaatgatttcattaagaattgtcatatcttggaatag

SEQ ID NO: 58 - protein, 533 aa, Arabidopsis thaliana
MQLHISPSMRSITISSSNEFIDLMKIKVAARHISYRTLFHTILILAFLLPFVFILTAVVTLEGVNKCSSFDCFGRRLGPRLLGRIDDSEQRLVRDFYKILNEVSTQEIPDGLKLPESFSQLVSDMKNNHYDAKTFALVFRAMVEKFERDLRESKFAELMNKHFAASSIPKGIHCLSLRLTDEYSSNAHARRQLPSPELLPVLSDNAYHHFVLATDNILAASVVVSSAVQSSSKPEKIVFHVITDKKTYAGMHSWFALNSVAPAIVEVKSVHQFDWLTRENVPVLEAVESHNSIRNYYHGNHIAGANLSETTPRTFASKLQSRSPKYISLLNHLRIYLPELFPNLDKVVFLDDDIVIQKDLSPLWDIDLNGKVNGAVETCRGEDVWVMSKRLRNYFNFSHPLIAKHLDPEECAWAYGMNIFDLRTWRKTNIRETYHSWLKENLKSNLTMWKLGTLPPALIAFKGHVQPIDSSWHMLGLGYQSKTNLENAKKAAVIHYNGQSKPWLEIGFEHLRPFWTKYVNYSNDFIKNCHILE

SEQ ID NO: 59 - DNA, 1599 bp, Arabidopsis thaliana
atgcagcttcacatatcgccgagtatgagaagcattacgatttcgagcagcaatgagtttattgacttgatgaagatcaaggtcgcagctcgtcacatctcttaccgaactctcttccacaccatcttaatcctcgctttcttgttgccttttgttttcattctcaccgctgttgttacccttgagggtgtcaacaaatgctcctccattgattgtttagggaggcggataggtccacgtcttcttggtagggtagatgattcagagagactagctagagacttttataaaattctaaacgaagtaagcactcaagaaattccagatggtttgaagcttccaaattcttttagtcaacttgtttccgatatgaagaataaccactatgatgcaaaaacatttgctcttgtgctgcgagccatgatggagaagtttgaacgtgatatgagggaatcgaaatttgcagaacttatgaacaagcactttgcagcaagttccattcccaaaggcattcattgtctctctctaagactgacagatgaatattcctccaatgctcatgctcgaagacagcttccttcaccagagtttctccctgttctttcagataatgcttaccaccactttattttgtccacggacaatattttggctgcctcagttgtggtctcatccgctgttcagtcatcttcaaaacccgagaaaattgtctttcacatcattacagacaagaaaacctatgcgggtatgcattcatggtttgcgcttaattctgttgcaccagcaattgttgaggttaaaggtgttcatcagtttgactggttgacgagagagaatgttccggttttggaagctgtggaaagccataatggtgtcagggactattatcatgggaatcatgtcgctggggcaaacctcaccgaaacaactcctcgaacatttgcttcaaaattgcagtctagaagtccaaaatacatatctttgctcaaccatcttagaatatatataccagagcttttcccgaacttggacaaggtggttttcttagacgatgatatagttgtccagggagacttaactccactttgggatgttgacctcggtggtaaggtcaatggggcagtagagacttgcaggggtgaagatgaatgggtgatgtcaaagcgtttaaggaactacttcaatttctctcacccgctcatcgcaaagcatttagatcctgaagaatgtgcttgggcatatggtatgaatatcttcgatctacaagcttggaggaaaacaaatatcagagaaacgtatcactcttggcttagagagaatctaaagtcaaatctgacaatgtggaaacttggaaccttgcctcctgctcttatcgcgttcaagggtcacgtacacataatagactcgtcatggcatatgctaggattaggctaccagagcaagaccaacatagaaaatgtgaagaaagcagcagtgatccactacaatgggcagtcaaagccatggctggagattggtttcgagcatctgcggccattctggaccaaatacgtcaactactcaaatgatttcatcaagaactgtcacatattggagtag

SEQ ID NO: 60 - protein, 532 aa, Arabidopsis thaliana
MQLHISPSMRSITISSSNEFIDLMKIKVAARHISYRTLFHTILILAFLLPFVFILTAVVTLEGVNKCSSIDCLGRRIGPRLLGRVDDSERLARDFYKILNEVSTQEIPDGLKLPNSFSQLVSDMKNNHYDAKTFALVLRAMMEKFERDMRESKFAELMNKHFAASSIPKGIHCLSLRLTDEYSSNAHARRQLPSPEFLPVLSDNAYHHFILSTDNILAASVVVSSAVQSSSKPEKIVFHIITDKKTYAGMHSWFALNSVAPAIVEVKGVHQFDWLTRENVPVLEAVESHNGVRDYYHGNHVAGANLTETTPRTFASKLQSRSPKYISLLNHLRIYIPELFPNLDKVVFLDDDIVVQGDLTPLWDVDLGGKVNGAVETCRGEDEWVMSKRLRNYFNFSHPLIAKHLDPEECAWAYGMNIFDLQAWRKTNIRETYHSWLRENLKSNLTMWKLGTLPPALIAFKGHVHIIDSSWHMLGLGYQSKTNIENVKKAAVIHYNGQSKPWLEIGFEHLRPFWTKYVNYSNDFIKNCHILE

SEQ ID NO: 61 - skipped

SEQ ID NO: 62 - protein, 528 aa, Populus trichocarpa
MRSITISSSSNNGFIDLMKIKVAARHISYRTLFHTILILAFLLPFVFILTALVTLEGVNKCSSFDCLGRRLGPRLLGRVDDSGRLVKDFYKILNQVKNEEIPDGVKLPASFSHLVSEMKNNQYDARTFAFMLRAMMEKLEREIRESKFSELMNKHFAASSIPKSIHCLSLRLTDEYSSNAHARKQLPSPEFLPLLSDNSYHHFVLSTDNILAASVVVTSTIQSSLKPDNIVFHIITDKKTYAGMHSWFALNPVSPAIVEVKGVHQFDWLTRENVPVLEAVENHNGIRNYYHGNHIAGANLSDTTPRRFASKLQARSPKYISILNHLRIYIPELFPSLDKVVFLDDDVVIQRDLSPLWEIDLKGKVNGAVETCKGEDEWVMSKHFKNYFNFSHPLIAKNLDPDECAWAYGMNIFDLRAWRKTNIRETYHSWLKENLKSNLTMWKLGTLPPALIAFKGHVHPIDPSWHMLGLGYQNKTNIESVKKAAVIHYNGQAKPWLEIGFEHLRPFWTKYVNYSNDFIRNCHILDSV

SEQ ID NO: 63 - skipped

SEQ ID NO: 64 - protein, 528 aa, Populus trichocarpa
MRSITISSSGNNGFIDSMKIKVAARHISYRTLFHTILILAFLLPFVFILTALVTLEGVNKCSSFDCLGRRLGPRLLGRVDDSGRLVKDFYKILNQVKNEEIPDGVKLPASFNHLVSEMKNNQYDARTFAFMLRAMMEKLEREIRESKFAELMNKHFAASSIPKSIHCLSLRLTDEYSSNAHARTQLPSPEFLPLLSDNSYHHFVLSTDNILAASVVVTSTVQSSLKPDRIVFHIITDKKTYAGMHSWFALNPASPAIVEVKGVHQFDWLTRENVPVLEAVENHNGIRDYYHGNHIAGANLSDTTPRRFASKLQARSPKYISLLNHLRIYIPELFPNLDKVVFLDDDVVIQHDLSPLWEIDLQGKVNGAVETCKGEDEWVMSKHLKNYFNFSHPLIAKNLDPDECAWAYGMNIFDLHAWRNTNIRETYHSWMKENLKSNLTMWKLGTLPPSLIAFKGHVHPIDPFWHMLGLGYQNNTNIESVKKAAVIHYNGQSKPWLEIGFEHLRPFWTKYVNYSNDFIRNCHILDSV

SEQ ID NO: 65 - DNA, 1623 bp, Arabidopsis thaliana
atgaagttttacatatcagcgacggggattaagaaggttacgatatcaaatcccggcgtcggaatcggtaaaggaagcggaggatgtgcggctgcagcggcggcgttagcagcgcggagattctctagtcgcacgttgttactgttgctgctgctgctcgctatcgtcctcccttttatcttcgtcaggttcgcgtttctcgtcctcgaatctgcctccgtttgcgattcaccactcgattgcatgggactcagacttttccgtgggggcgacacatctctgaaaattggggaagagttgacacgggctctagtggaagagacgacagatcatcaggacgttaatggaagaggaacgaagggatcattggagtcattcgacgaccttgttaaggagatgacgttaaaacgccgtgacataagggcgtttgcttccgtgactaagaagatgctgttgcagatggaacgtaaagtccaatcagcgaaacatcatgagttagtgtactggcatttagcctctcacggtattcctaaaagcctccattgcctttccctcagattaactgaagagtactctgtaaatgcaatggctcgaatgcgtttgcctccgcctgagtccgtatcacgtctgaccgacccatcttttcatcatattgtcctcctgactgacaatgtccttgctgcctctgtcgtcatatcgtctactgtacaaaacgctgtgaatcccgagaagtttgtctttcatattgttaccgataagaaaacctatacccctatgcatgcttggtttgctatcaactctgcttcatcaccagttgttgaagtaaagggacttcatcagtatgattggcctcaagaagtgaacttcaaagttagagagatgctggacattcaccgcttaatttggagacgacattatcaaaatttgaaagactctgattttagttttgttgagggtactcatgagcagtccttgcaagctctaaatcctagctgccttgcccttttgaaccatcttcgcatttacattcccaagctttttccagatctcaacaagatagtgttgttggatgatgatgtagtagtacagagcgatctttcgtctttatgggaaacggatctcaacggtaaagttgttggtgctgtcgttgattcgtggtgcggagacaactgttgccccggaagaaaatacaaagactatttcaacttctcacatcctttgatctcatcaaacttagttcaagaagactgtgcttggctttctggtatgaatgtctttgatctcaaagcctggagacaaaccaatattactgaagcttactctacatggctaagactcagtgttaggtcaggactacaattatggcaaccaggggctttaccaccgacattacttgctttcaaaggacttacacagtctcttgaaccatcatggcacgtcgctggactaggttctcgatccgtaaaatcccctcaagagattctgaaatctgcttcggttttacatttcagcggtccagcaaaaccgtggctagagatcagtaaccctgaggtacgatctctttggtatagatacgtaaattcctccgacatcttcgttagaaaatgcaaaatcatgaactga

SEQ ID NO: 66 - protein, 540 aa, Arabidopsis thaliana
MKFYISATGIKKVTISNPGVGIGKGSGGCAAAAAALAARRFSSRTLLLLLLLLAIVLPFIFVRFAFLVLESASVCDSPLDCMGLRLFRGGDTSLKIGEELTRALVEETTDHQDVNGRGTKGSLESFDDLVKEMTLKRRDIRAFASVTKKMLLQMERKVQSAKHHELVYWHLASHGIPKSLHCLSLRLTEEYSVNAMARMRLPPPESVSRLTDPSFHHIVLLTDNVLAASVVISSTVQNAVNPEKFVFHIVTDKKTYTPMHAWFAINSASSPVVEVKGLHQYDWPQEVNFKVREMLDIHRLIWRRHYQNLKDSDFSFVEGTHEQSLQALNPSCLALLNHLRIYIPKLFPDLNKIVLLDDDVVVQSDLSSLWETDLNGKVVGAVVDSWCGDNCCPGRKYKDYFNFSHPLISSNLVQEDCAWLSGMNVFDLKAWRQTNITEAYSTWLRLSVRSGLQLWQPGALPPTLLAFKGLTQSLEPSWHVAGLGSRSVKSPQEILKSASVLHFSGPAKPWLEISNPEVRSLWYRYVNSSDIFVRKCKIMN

SEQ ID NO: 67 - skipped

SEQ ID NO: 68 - protein, 531 aa, Populus trichocarpa
MKFYISTTGIKRVTISTTNSSAKGSTVATRRITRRTFLPVVLLLSIVLPFLFVRIAFLVLESASACNSALDCIGWGLLGGSEASLLREELTRALMEAKEGRGTNDGDYRTEGSTESFNVLVNEMTSNQQDIKTFAFRTKAMLSMMELKVQSAREQESINWHLASHGVPKSLHCLCLKLAEEYAVNAMARSHLPPPEYVSRLTDPSFHHVVLLTDNVLAASVVISSTVQHSANPEKLVFHIVTDKKTYIPMNAWFAINPIKSAAVEVKGLHQYDWSHEVNVHVKEMLEIHRLIWSHYNDNLRNANFQHEGVNRRSLEALTPSCLSLLNHLRIYIPELFPDLNKIVFLDEDVVVQHDMSSLWELDLNKKVVGAVVDSWCGDNCCPGKKYKDYLNFSYPIISSNFDHDRCVWLYGVNVFDLEAWRRVKITTNYHKWLKHNLNFGMELWQPGVHPPALLAFEGQVHPIDPSWHVGGLGYRPPQAHNIKMLGDAAVLHFSGPAKPWLDIGFPELRSLWNRHVNFSDKFIRKCRILG

SEQ ID NO: 69 - DNA, 27 bp, Artificial Sequence
  Description of Artificial Sequence: Synthetic primer
atggcgctaaagcgagggctatctgga

SEQ ID NO: 70 - DNA, 27 bp, Artificial Sequence
  Description of Artificial Sequence: Synthetic primer
tcgttcttgtttttcaattttgcaatc

SEQ ID NO: 71 - DNA, 27 bp, Artificial Sequence
  Description of Artificial Sequence: Synthetic primer
atgactgatgcttgttgtttgaaggga

SEQ ID NO: 72 - DNA, 27 bp, Artificial Sequence
  Description of Artificial Sequence: Synthetic primer
atcagagaagagagcgtagtggtaaag

SEQ ID NO: 73 - DNA, 27 bp, Artificial Sequence
  Description of Artificial Sequence: Synthetic primer
atgtcggtggagccattttagagtcac

SEQ ID NO: 74 - DNA, 27 bp, Artificial Sequence
  Description of Artificial Sequence: Synthetic primer
ttgaaggaaggtcagcatcagaggttg

SEQ ID NO: 75 - DNA, 27 bp, Artificial Sequence
  Description of Artificial Sequence: Synthetic primer
atgatggtgaagcttcgcaatcttgtt

SEQ ID NO: 76 - DNA, 27 bp, Artificial Sequence
  Description of Artificial Sequence: Synthetic primer
ggagcatagcacgtagcttcttgacca

SEQ ID NO: 77 - DNA, 27 bp, Artificial Sequence
  Description of Artificial Sequence: Synthetic primer
atgaatcaagttcgtcgttggcagagg

SEQ ID NO: 78 - DNA, 27 bp, Artificial Sequence
  Description of Artificial Sequence: Synthetic primer
tgtgaaaggcacggctgaccttgtata

SEQ ID NO: 79 - DNA, 27 bp, Artificial Sequence
  Description of Artificial Sequence: Synthetic primer
atgaaacaaattcgtcgatggcagagg

SEQ ID NO: 80 - DNA, 27 bp, Artificial Sequence
  Description of Artificial Sequence: Synthetic primer
cttctgtgttataattcatggcacgga

SEQ ID NO: 81 - DNA, 27 bp, Artificial Sequence
  Description of Artificial Sequence: Synthetic primer
atgaaaggcggaggcggtggtggagga

SEQ ID NO: 82 - DNA, 29 bp, Artificial Sequence
  Description of Artificial Sequence: Synthetic primer
cttcacaagttctccaagtttcatcacca

SEQ ID NO: 83 - DNA, 27 bp, Artificial Sequence
  Description of Artificial Sequence: Synthetic primer
atggctaatcaccaccgacttttacgc

SEQ ID NO: 84 - DNA, 27 bp, Artificial Sequence
  Description of Artificial Sequence: Synthetic primer
gtaaagattcggatcctcgagctcccg

SEQ ID NO: 85 - DNA, 27 bp, Artificial Sequence
  Description of Artificial Sequence: Synthetic primer
atgggcaacgcatatatgcagaggacg

SEQ ID NO: 86 - DNA, 27 bp, Artificial Sequence
  Description of Artificial Sequence: Synthetic primer
caccttcatggctgcgagattcatccg

SEQ ID NO: 87 - DNA, 27 bp, Artificial Sequence
  Description of Artificial Sequence: Synthetic primer
atgagaaggagaggaggggatagtttc

SEQ ID NO: 88 - DNA, 27 bp, Artificial Sequence
  Description of Artificial Sequence: Synthetic primer
ccacaacagaagtagcaataatgttat

SEQ ID NO: 89 - DNA, 27 bp, Artificial Sequence
  Description of Artificial Sequence: Synthetic primer
atgaggcggtggccggtggatcaccgg

SEQ ID NO: 90 - DNA, 27 bp, Artificial Sequence
  Description of Artificial Sequence: Synthetic primer
ctcatctgccagttcatggcgagatgg

SEQ ID NO: 91 - DNA, 27 bp, Artificial Sequence
  Description of Artificial Sequence: Synthetic primer
atgcagttacatatatctccgagcttg

SEQ ID NO: 92 - DNA, 27 bp, Artificial Sequence
  Description of Artificial Sequence: Synthetic primer
tagccacaaccgaagctgcaagaatat

SEQ ID NO: 93 - DNA, 27 bp, Artificial Sequence
  Description of Artificial Sequence: Synthetic primer
atgcagcttcacatatcgcctagcatg

SEQ ID NO: 94 - DNA, 27 bp, Artificial Sequence
  Description of Artificial Sequence: Synthetic primer
ttcttgtctgtgataacatggaagaca

SEQ ID NO: 95 - DNA, 27 bp, Artificial Sequence
  Description of Artificial Sequence: Synthetic primer
atgcagcttcacatatcgcctagcatg

SEQ ID NO: 96 - DNA, 27 bp, Artificial Sequence
  Description of Artificial Sequence: Synthetic primer
cagcagatgagaccacaaccgatgcag

SEQ ID NO: 97 - DNA, 29 bp, Artificial Sequence
  Description of Artificial Sequence: Synthetic primer
atgaagttttacatatcagcgacggggat

SEQ ID NO: 98 - DNA, 29 bp, Artificial Sequence
  Description of Artificial Sequence: Synthetic primer
cgagccattgcatttacagagtactcttc

SEQ ID NO: 99 - DNA, 25 bp, Artificial Sequence
  Description of Artificial Sequence: Synthetic primer
ccatgtctccggctaaagttgatac

SEQ ID NO: 100 - DNA, 26 bp, Artificial Sequence
  Description of Artificial Sequence: Synthetic primer
cagcacgaatgtcaacaatgaaaaca

SEQ ID NO: 101 - DNA, 29 bp, Artificial Sequence
  Description of Artificial Sequence: Synthetic primer
tcagaagaagtttgaactgagttagccac

SEQ ID NO: 102 - DNA, 29 bp, Artificial Sequence
  Description of Artificial Sequence: Synthetic primer
atgtttaacaagcccaataaggcataatc

SEQ ID NO: 103 - DNA, 26 bp, Artificial Sequence
  Description of Artificial Sequence: Synthetic primer
tttgaaaactcagtcatagggaaata

SEQ ID NO: 104 - DNA, 26 bp, Artificial Sequence
  Description of Artificial Sequence: Synthetic primer
gaaggatgatttgctttgaaatagta

SEQ ID NO: 105 - DNA, 29 bp, Artificial Sequence
  Description of Artificial Sequence: Synthetic primer
accaggttaaagccattgtagagtgaaat

SEQ ID NO: 106 - DNA, 27 bp, Artificial Sequence
  Description of Artificial Sequence: Synthetic primer
atgtagcactactacctgcaaatcgtc

SEQ ID NO: 107 - DNA, 29 bp, Artificial Sequence
  Description of Artificial Sequence: Synthetic primer
gatcattataactttgttgcaaaagctgc

SEQ ID NO: 108 - DNA, 29 bp, Artificial Sequence
  Description of Artificial Sequence: Synthetic primer
aatgcggaggtacgtagtttaatccagtt

SEQ ID NO: 109 - DNA, 29 bp, Artificial Sequence
  Description of Artificial Sequence: Synthetic primer
taatgttgagatacagatatagtgcggcg

SEQ ID NO: 110 - DNA, 29 bp, Artificial Sequence
  Description of Artificial Sequence: Synthetic primer
aaaattcaaagctagctgaagtaaaagtg

SEQ ID NO: 111 - DNA, 29 bp, Artificial Sequence
  Description of Artificial Sequence: Synthetic primer
ttatctaagggtgaaaagaacacaagggt

SEQ ID NO: 112 - DNA, 29 bp, Artificial Sequence
  Description of Artificial Sequence: Synthetic primer
acattgagattgctgggtaattaagtgaa

SEQ ID NO: 113 - DNA, 25 bp, Artificial Sequence
  Description of Artificial Sequence: Synthetic primer
cagggaagaacaagtgattgtttca

SEQ ID NO: 114 - DNA, 26 bp, Artificial Sequence
  Description of Artificial Sequence: Synthetic primer
gaaatgcatgatacctttgatgaaga

SEQ ID NO: 115 - DNA, 29 bp, Artificial Sequence
  Description of Artificial Sequence: Synthetic primer
catagtcaacgttaacacccatttgactt

SEQ ID NO: 116 - DNA, 29 bp, Artificial Sequence
  Description of Artificial Sequence: Synthetic primer
ctcttaagccgattcgatacgaaaataag

SEQ ID NO: 117 - DNA, 28 bp, Artificial Sequence
  Description of Artificial Sequence: Synthetic primer
atatcaaggtcccaaaggggagataagt

SEQ ID NO: 118 - DNA, 29 bp, Artificial Sequence
  Description of Artificial Sequence: Synthetic primer
ctcaagagaagctttgatgtgtagaatcc

SEQ ID NO: 119 - DNA, 25 bp, Artificial Sequence
  Description of Artificial Sequence: Synthetic primer
ttcggatacatctctctgcaaaacc

SEQ ID NO: 120 - DNA, 25 bp, Artificial Sequence
  Description of Artificial Sequence: Synthetic primer
cttgcaccagattgaacctaaatgg

SEQ ID NO: 121 - DNA, 29 bp, Artificial Sequence
  Description of Artificial Sequence: Synthetic primer
gatcaaagagaagtttaatcccaaagcat

SEQ ID NO: 122 - DNA, 29 bp, Artificial Sequence
  Description of Artificial Sequence: Synthetic primer
taattggagtcaaaacttgagagcaagag

SEQ ID NO: 123 - DNA, 29 bp, Artificial Sequence
  Description of Artificial Sequence: Synthetic primer
tctcttctaatgatctaatcccacaataa

SEQ ID NO: 124 - DNA, 29 bp, Artificial Sequence
  Description of Artificial Sequence: Synthetic primer
ggtttgttaatcagatccgtgtaattcct

SEQ ID NO: 125 - DNA, 29 bp, Artificial Sequence
  Description of Artificial Sequence: Synthetic primer
tctcttctaatgatctaatcccacaataa

SEQ ID NO: 126 - DNA, 29 bp, Artificial Sequence
  Description of Artificial Sequence: Synthetic primer
ggtttgttaatcagatccgtgtaattcct

SEQ ID NO: 127 - DNA, 25 bp, Artificial Sequence
  Description of Artificial Sequence: Synthetic primer
acagcctgttgtaacaaagcccata

SEQ ID NO: 128 - DNA, 25 bp, Artificial Sequence
  Description of Artificial Sequence: Synthetic primer
ctcgctgtcttcaccttatccttca

SEQ ID NO: 129 - DNA, 29 bp, Artificial Sequence
  Description of Artificial Sequence: Synthetic primer
tctctgataatgtcattgctgtgtctgtt

SEQ ID NO: 130 - DNA, 29 bp, Artificial Sequence
  Description of Artificial Sequence: Synthetic primer
tcatgtttccattgtaatgaatcactcct

SEQ ID NO: 131 - DNA, 29 bp, Artificial Sequence
  Description of Artificial Sequence: Synthetic primer
acacagcttaaaatccagaagttgaaaga

SEQ ID NO: 132 - DNA, 29 bp, Artificial Sequence
  Description of Artificial Sequence: Synthetic primer
agttaaacaatggacttaccaggttctgc

SEQ ID NO: 133 - DNA, 28 bp, Artificial Sequence
  Description of Artificial Sequence: Synthetic primer
ctcttctttctcattctctccaaagctg

SEQ ID NO: 134 - DNA, 27 bp, Artificial Sequence
  Description of Artificial Sequence: Synthetic primer
atgagaaatcctcgaacttctgaacct

SEQ ID NO: 135 - DNA, 29 bp, Artificial Sequence
  Description of Artificial Sequence: Synthetic primer
atgggtttttaaccaatacccgaattact

SEQ ID NO: 136 - DNA, 29 bp, Artificial Sequence
  Description of Artificial Sequence: Synthetic primer
agcaagagcaatctgatcattaacttgac

SEQ ID NO: 137 - DNA, 29 bp, Artificial Sequence
  Description of Artificial Sequence: Synthetic primer
ccaaatcaaacgaaatgaaagtagacaaa

SEQ ID NO: 138 - DNA, 29 bp, Artificial Sequence
  Description of Artificial Sequence: Synthetic primer
cgaacattagcagttataaacactcaccc

SEQ ID NO: 139 - DNA, 25 bp, Artificial Sequence
  Description of Artificial Sequence: Synthetic primer
tatttcgtttgatgaggctaaaccg

SEQ ID NO: 140 - DNA, 25 bp, Artificial Sequence
  Description of Artificial Sequence: Synthetic primer
tttcgatcagacggttatcgatgtt

SEQ ID NO: 141 - DNA, 21 bp, Artificial Sequence
  Description of Artificial Sequence: Synthetic primer
ggtttgcttcttgcttccgct

SEQ ID NO: 142 - DNA, 23 bp, Artificial Sequence
  Description of Artificial Sequence: Synthetic primer
tttgggacattgacatgaatgga

SEQ ID NO: 143 - DNA, 26 bp, Artificial Sequence
  Description of Artificial Sequence: Synthetic primer
ttttagtgagaatcgaatgttttgtc

SEQ ID NO: 144 - DNA, 25 bp, Artificial Sequence
  Description of Artificial Sequence: Synthetic primer
cttcaacataaagccaaatcctaaa

SEQ ID NO: 145 - DNA, 29 bp, Artificial Sequence
  Description of Artificial Sequence: Synthetic primer
aaaaggcttgatttttcttcttctcctct

SEQ ID NO: 146 - DNA, 29 bp, Artificial Sequence
  Description of Artificial Sequence: Synthetic primer
ccttaacttgatagttgaacaaaatgcca

SEQ ID NO: 147 - DNA, 27 bp, Artificial Sequence
  Description of Artificial Sequence: Synthetic primer
ttaagtctccctggacaactatatcat

SEQ ID NO: 148 - DNA, 25 bp, Artificial Sequence
  Description of Artificial Sequence: Synthetic primer
caattgtcaagttggtttcttttct

SEQ ID NO: 149 - DNA, 21 bp, Artificial Sequence
  Description of Artificial Sequence: Synthetic primer
ttgggtccgctactgatctga

SEQ ID NO: 150 - DNA, 22 bp, Artificial Sequence
  Description of Artificial Sequence: Synthetic primer
gcagtgatccactacaatgggc

SEQ ID NO: 151 - DNA, 28 bp, Artificial Sequence
  Description of Artificial Sequence: Synthetic primer
agcactatgtgcaagtgttgagattttt

SEQ ID NO: 152 - DNA, 29 bp, Artificial Sequence
  Description of Artificial Sequence: Synthetic primer
tgtttttgatgaactgatagtggagatca

SEQ ID NO: 153 - DNA, 25 bp, Artificial Sequence
  Description of Artificial Sequence: Synthetic primer
ttttctaaagaagccaagcggacat

SEQ ID NO: 154 - DNA, 27 bp, Artificial Sequence
  Description of Artificial Sequence: Synthetic primer
tgttatccacagctgacaatgtttttg

SEQ ID NO: 155 - DNA, 29 bp, Artificial Sequence
  Description of Artificial Sequence: Synthetic primer
tggcatctatagtaatccatacgacgatt

SEQ ID NO: 156 - DNA, 29 bp, Artificial Sequence
  Description of Artificial Sequence: Synthetic primer
ttgaatgctatgtgcttgtcatctttaat

SEQ ID NO: 157 - DNA, 22 bp, Artificial Sequence
  Description of Artificial Sequence: Synthetic primer
tggttcacgtagtgggccatcg

SEQ ID NO: 158 - DNA, 22 bp, Artificial Sequence
  Description of Artificial Sequence: Synthetic primer
gcgtggaccgcttgctgcaact

SEQ ID NO: 159 - DNA, 28 bp, Artificial Sequence
  Description of Artificial Sequence: Synthetic primer
ggtgatggttcacgtagtgggccatcgc

SEQ ID NO: 160 - DNA, 27 bp, Artificial Sequence
  Description of Artificial Sequence: Synthetic primer
atgcagcttcacatatcgcctagcatg

SEQ ID NO: 161 - DNA, 27 bp, Artificial Sequence
  Description of Artificial Sequence: Synthetic primer
cagcagatgagaccacaaccgatgcag

SEQ ID NO: 162 - DNA, 27 bp, Artificial Sequence
  Description of Artificial Sequence: Synthetic primer
ttaagtctccctggacaactatatcat

SEQ ID NO: 163 - DNA, 25 bp, Artificial Sequence
  Description of Artificial Sequence: Synthetic primer
caattgtcaagttggtttcttttct

SEQ ID NO: 164 - DNA, 21 bp, Artificial Sequence
  Description of Artificial Sequence: Synthetic primer
ttgggtccgctactgatctga

SEQ ID NO: 165 - DNA, 22 bp, Artificial Sequence
  Description of Artificial Sequence: Synthetic primer
gcagtgatccactacaatgggc

SEQ ID NO: 166 - DNA, 22 bp, Artificial Sequence
  Description of Artificial Sequence: Synthetic primer
caaggcagtctgcagatattac

SEQ ID NO: 167 - DNA, 20 bp, Artificial Sequence
  Description of Artificial Sequence: Synthetic primer
cttatgcaaccttcccttcg

SEQ ID NO: 168 - DNA, 20 bp, Artificial Sequence
  Description of Artificial Sequence: Synthetic primer
agtgtctggatcggtggttc

SEQ ID NO: 169 - DNA, 20 bp, Artificial Sequence
  Description of Artificial Sequence: Synthetic primer
atcatactcggccttggaga

SEQ ID NO: 170 - protein, 9 aa, Artificial Sequence
  Description of Artificial Sequence: Synthetic peptide
  MOD_RES (2)..(3): Any amino acid
  MOD_RES (5)..(6): Any amino acid
HXXGXXKPW

SEQ ID NO: 171 - protein, 9 aa, Artificial Sequence
  Description of Artificial Sequence: Synthetic peptide
  MOD_RES (2)..(2): Any amino acid
  MOD_RES (4)..(4): Any amino acid
  MOD_RES (8)..(8): Any amino acid
DXDXVVQXD

SEQ ID NO: 172 - protein, 11 aa, Artificial Sequence
  Description of Artificial Sequence: Synthetic peptide
  MOD_RES (3)..(7): Any amino acid
WHXXXXXGLGY

SEQ ID NO: 173 - protein, 8 aa, Artificial Sequence
  Description of Artificial Sequence: Synthetic peptide
  MOD_RES (3)..(4): Any amino acid
  MOD_RES (6)..(7): Any amino acid
LPXXLXXF

SEQ ID NO: 174 - protein, 15 aa, Artificial Sequence
  Description of Artificial Sequence: Synthetic peptide
  MOD_RES (2)..(2): Any amino acid
  MOD_RES (4)..(6): Any amino acid
  MOD_RES (9)..(10): Any amino acid
  MOD_RES (12)..(14): Any amino acid
CXWXXXMNXXDXXXW

SEQ ID NO: 175 - protein, 9 aa, Artificial Sequence
  Description of Artificial Sequence: Synthetic peptide
  MOD_RES (4)..(4): Any amino acid
  MOD_RES (7)..(8): Any amino acid
RFYXPEXXP

(19) United States
(12) Patent Application Publication
(10) Pub. No.: US 2013/0102022 A1
(43) Pub. Date: Apr. 25, 2013

(54) PLANTS WITH ALTERED CELL WALL BIOSYNTHESIS AND METHODS OF USE

(71) Applicants: Debra A. Mohnen, Athens, GA (US); Ajaya Kumar Biswal, Athens, GA (US); Zhangying Hao, Athens, GA (US); Kimberly D. Hunt, Athens, GA (US); Ivana Gelineo-Albersheim, Athens, GA (US); Irina Kataeva, Athens, GA (US); Michael W.W. Adams, Athens, GA (US)

(72) Inventors: Debra A. Mohnen, Athens, GA (US); Ajaya Kumar Biswal, Athens, GA (US); Zhangying Hao, Athens, GA (US); Kimberly D. Hunt, Athens, GA (US); Ivana Gelineo-Albersheim, Athens, GA (US); Irina Kataeva, Athens, GA (US); Michael W.W. Adams, Athens, GA (US)

(73) Assignee: UNIVERSITY OF GEORGIA RESEARCH FOUNDATION, INC., Athens, GA (US)

(21) Appl. No.: 13/638,143

(22) PCT Filed: Apr. 15, 2011
(86) PCT No.: PCT/US2011/032733

§ 371 Date: Dec. 21, 2012

Related U.S. Application Data
(60) Provisional application No. 61/342,618, filed on Apr. 16, 2010.
(60) Provisional application No. 61/397,951, filed on Jun. 18, 2010.
(60) Provisional application No. 61/399,254, filed on Jul. 9, 2010.

(51) Int. Cl. C12N 15/82; D21B 1/04

(52) U.S. Cl. CPC C12N 15/8243; D21B 1/04
USPC 435/29; 435/165; 800/290; 800/260; 800/298; 162/24; 162/100; 241/28

(57) ABSTRACT

Provided herein are plants having altered expression of a GAUT polypeptide. Such plants have phenotypes that may include decreased recalcitrance, increased growth, decreased lignin content, or a combination thereof. Also provided herein are methods of making and using such plants.

### CROSS-REFERENCE TO RELATED APPLICATIONS

This application claims the benefit of U.S. Provisional Application Ser. No. 61/342,618, filed Apr. 16, 2010, U.S. Provisional Application Ser. No. 61/397,951, filed Jun. 18, 2010, and 61/399,254, filed Jun. 9, 2010, each of which is incorporated by reference herein.

### GOVERNMENT FUNDING

The present invention was made with government support under MCB awards 0313509 and 0646109 from the NSF, awards 2003-35318-15377 and 2006-35318-17301 from the USDA, and award DE-FG02-93-ER20097 from the DOE. The Government has certain rights in this invention.

### BACKGROUND

There is increasing interest in the use of biomass for biofuel production as an environmental friendly and socio-economically responsible fuel alternative. Bioenergy originates in biomass generated by CO₂ fixation by land plants. Approximately 70% of plant biomass is estimated to be present in plant cell wall (Pauly and Keegstra, 2008, Plant J., 54:559-568). As only 2% of plant cell wall-based biomass is currently being used, there is a great opportunity to use this valuable resource as a raw material for biofuels (Schubert, 2006, Nat. Biotechnol., 24:777-784; Pauly and Keegstra, 2008, Plant J., 54:559-568)

The plant cell wall provides mechanical support to the plant and contributes to plant growth and development. Carbohydrates, proteins and phenolic compounds are the major components in the plant cell wall with cellulose, hemicellulose and pectin comprising the major polysaccharides in the wall. Pectins are enriched in the primary wall of dicot plants, are essential for plant growth, development, signaling, and cell adhesion and have diverse structural characteristics that greatly contribute to wall function (Mohnen, 2008, Curr. Opin. Plant Biol., 11:1-12). There are three major classes of pectin: homogalacturonan (HG), rhamnogalacturonan-I (RG-I), and rhamnogalacturonan-II (RG-II). HG is the most abundant pectic polysaccharide and is a homopolymer of α-1,4-linked galacturonic acid (GalA) that may be modified by O -acetylation at the O-2 or O-3 and methylesterification at C-6. HG comprises about 65% of pectin in the primary walls of dicots (Mohnen, 2008, Curr. Opin. Plant Biol., 11:1-12). RG-I consists of a backbone of alternating α-1,4-linked GalA and α-1,2-rhamnose and represents ˜20-35% of pectin. The L-rhamnose residues of the RG-I backbone have side chains which are either linear or branched and largely composed of β-D-galactose and α-L-arabinose residues. There is a large variation in RG-I structures in different groups of plants (Mohnen, 2008, Curr. Opin. Plant Biol., 11:1-12). The most complex pectic-polysaccharide is RG-II. RG-II molecule consists of an HG backbone of approximately seven to nine GalA residues which is branched by four highly conserved side chains. The side chains of RG-II consist of at least 12 different types of glycosyl residues including several types of rare sugars with more than 20 different linkages to form a structure that is highly conserved in all vascular plants. RG-II comprises about 10% of total pectin (O'Neill et al., 2004, Annual Rev. Plant Biol., 55:109-139; Mohnen, 2008, Curr. Opin. Plant Biol., 11:1-12).

Mohnen and coworkers identified an Arabidopsis homogalactronanan α-1,4-galacturonosyltransferase (HG:α1,4GalAT), called GAUT1 (galacturonosyltransferase 1) (Sterling et al., 2006, Proc. Natl. Acad. Sci. USA, 103:5236-41), that is involved in HG synthesis. In Arabidopsis, the GAUT1-related gene family is made up of 15 GAUTs genes with 56-100% sequence similarity to GAUT1 (Sterling et al., 2006, Proc. Natl. Acad. Sci. USA, 103:5236-41). GAUT genes have been shown to be of importance in plant growth and development.

### SUMMARY OF THE INVENTION

The goal of using bioenergy crops for bio-ethanol production in the United States is well established. However, cost effectiveness is one of the major limitations for this industry and therefore many researchers are working to tackle this problem. The major barrier is the cost of the bacterial and fungal enzymes needed to degrade the plant cell wall and the pretreatment conditions required to deconstruct the wall. Described herein is the identification of recalcitrance genes which can be modified to produce genetically modified plant cell walls from which sugars can more easily be released, and thus, which would serve as raw materials for bio-ethanol industry.

Provided herein are methods for using plants. In one embodiment the plant is a transgenic plant. In one embodiment the method includes processing a transgenic plant to result in pulp, wherein the transgenic plant includes decreased or increased expression of a coding region encoding a GAUT polypeptide compared to a control plant. In one embodiment, the GAUT polypeptide may be selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide. The processing may include a physical pretreatment, a chemical pretreatment, or a combination thereof. The method may include hydrolyzing the processed pulp, and optionally contacting the processed pulp with an ethanologenic microbe, such as a eukaryote. The method may also include obtaining a metabolic product, such as ethanol, a diol, or an organic acid.

Also provided herein are methods for hydrolyzing a pulp. In one embodiment the pulp includes cells from a transgenic plant. In one embodiment the cells include a mutation in a coding region encoding GAUT polypeptide. In one embodiment, the GAUT polypeptide may be selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide. The hydrolyzing may include contacting the pulp with a composition that includes a cellulase under conditions suitable for hydrolysis. The hydrolyzed pulp may be contacted with an ethanologenic microbe, such as a eukaryote. Optionally, the method may include obtaining a metabolic product, such as ethanol, a diol, or an organic acid.

Also provided herein are methods for producing a metabolic product. The method may include contacting, under conditions suitable for the production of a metabolic product, a microbe with a composition that includes a pulp obtained from a transgenic plant, wherein the transgenic plant includes decreased or increased expression of a coding region encoding a GAUT polypeptide compared to a control plant. In one embodiment, the GAUT polypeptide may be selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide. The microbe may be an ethanologenic microbe, such as a eukaryote. The method may also include obtaining a metabolic product, such as ethanol, a diol, or an organic acid. The method may further include fermenting the pulp.

Also provided herein are methods for generating a transgenic plant having decreased recalcitrance, reduced lignification, increased growth, or the combination thereof, compared to a plant of substantially the same genetic background grown under the same conditions. The method may include transforming a cell of a plant with a polynucleotide to obtain a recombinant plant cell, generating a transgenic plant from the recombinant plant cell, wherein the transgenic plant has decreased or increased expression of a coding region encoding a GAUT polypeptide compared to a control plant. The transgenic plant may include a phenotype selected from decreased recalcitrance, reduced lignification, increased growth, or the combination thereof, compared to a control plant. The plant may be a dicot plant or a monocot plant. The method may further include breeding the transgenic plant with a second plant, wherein the second plant is transgenic or nontransgenic. The transgenic plant may be a woody plant, such as a member of the genus Populus. The method may further include screening the transgenic plant for decreased recalcitrance, reduced lignification, increased growth, or the combination thereof. The GAUT polypeptide may be selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide.

Also provided herein are transgenic plants that have decreased or increased expression of a coding region encoding a GAUT polypeptide compared to a control plant. In one embodiment the GAUT polypeptide may be selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT 12 polypeptide, a GAUT 13 polypeptide, a GAUT 14 polypeptide, or a GAUT15 polypeptide. In one embodiment the GAUT polypeptide is selected from a polypeptide having an amino acid sequence that has at least 80% sequence identity with SEQ ID NO: SEQ ID NO:2, SEQ ID NO:4, SEQ ID NO:6,

SEQ ID NO:8, SEQ ID NO:10, SEQ ID NO:12, SEQ ID NO:14, SEQ ID NO:16, SEQ ID NO:18, SEQ ID NO: 20, SEQ ID NO: 22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, and SEQ ID NO:66. The transgenic plant may include a phenotype selected from decreased recalcitrance, reduced lignification, increased growth, or the combination thereof. The plant may be a dicot or a monocot. The invention also includes (i) a part of a transgenic plant, such as a leaf, a stem, a flower, an ovary, a fruit, a seed, and a callus, (ii) the progeny of a transgenic plant, (iii) a wood obtained from a transgenic plant, and (iv) a pulp obtained from a transgenic plant.

Also provided herein are methods for measuring a change in recalcitrance of a plant. The methods include growing under suitable conditions a Caldicellulosiruptor saccharolyticus on material obtained from a first plant and a second plant, wherein the first plant is a transgenic plant described herein, and wherein the second plant is a control plant; and measuring (i) the time required for the C. saccharolyticus to reach stationary phase or (ii) the cell density after stationary phase is reached, wherein the C. saccharolyticus reaching stationary phase in shorter time or achieving a higher cell density when grown on the transgenic plant material indicates the transgenic plant has decreased recalcitrance compared to the control plant.

As used herein, the term “transgenic plant” refers to a plant that has been transformed to contain at least one modification to result in altered expression of a coding region. For example, a coding region in a plant may be modified to include a mutation to reduce transcription of the coding region or reduce activity of a polypeptide encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. The term “transgenic plant” includes whole plant, plant parts (stems, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the term “control plant” refers to a plant that is the same species as a transgenic plant, but has not been transfoimed with the same polynucleotide used to make the transgenic plant.

As used herein, the term “plant tissue” encompasses any portion of a plant, including plant cells. Plant cells include suspension cultures, callus, embryos, meristematic regions, callus tissue, leaves, roots, shoots, gametophytes, sporophytes, pollen, seeds and microspores. Plant tissues can be grown in liquid or solid culture, or in soil or suitable media in pots, greenhouses or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a polypeptide encoded by the coding region.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a polypeptide that may be encoded by the polynucleotide.

As used herein, “phenotype” refers to a distinguishing feature or characteristic of a plant which can be altered according to the present invention by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” as used herein refers to a modification of the natural nucleotide sequence of a coding region or an operably linked regulatory region made by deleting, substituting, or adding a nucleotide(s) in such a way that the polypeptide encoded by the modified nucleic acid is altered structurally and/or functionally, or the coding region is expressed at a decreased level.

As used herein, a “target coding region” and “target coding sequence” refer to a specific coding region whose expression is inhibited by a polynucleotide of the present invention. As used herein, a “target mRNA” is an mRNA encoded by a target coding region.

As used herein, the temrm “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

As used herein, a polypeptide may be “structurally similar” to a reference polypeptide if the amino acid sequence of the polypeptide possesses a specified amount of sequence similarity and/or sequence identity compared to the reference polypeptide. Thus, a polypeptide may be “structurally similar” to a reference polypeptide if, compared to the reference polypeptide, it possesses a sufficient level of amino acid sequence identity, amino acid sequence similarity, or a combination thereof.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. A polynucleotide described herein may be isolated. An “isolated” polynucleotide is one that has been removed from its natural environment. Polynucleotides that are produced by recombinant, enzymatic, or chemical techniques are considered to be isolated and purified by definition, since they were never present in a natural environment.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide.

As used herein, “recalcitrance” refers to the natural resistance of plant cell walls to microbial and/or enzymatic deconstruction.

Conditions that are “suitable” for an event to occur, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. The GAUT Protein Family of Arabidopsis, Poplar, and Rice. Phylogenetic analysis of the GAUT Family in Arabidopsis thaliana, Oryza sativa, and Populus trichocarpa. Alignment of the complete protein sequences of the GAUT family was carried out with ClustalX (Thompson et al., 1997, Nucleic Acids Res. 24, 4876 1882) using suggested parameters (Hall, B.G. 2004, Phylogenetic Trees Made Easy: A How-To Manual, 2nd ed, (Sunderland, M A: Sinauer Associates, Inc.), pp 29-30) for protein alignments. Bayesian analysis employing MrBayes (Huelsenbeck and Ronquist, 2001, Bioinformatics. 17, 754-755; Ronquist and Huelsenbeck, 2003, Bioinformatics, 19, 1574) was used to infer phylogenetic relationships between the members of the family and group the protein sequences into related clades. The analysis was carried out for 500 000 generations, using a mixture of amino acid transition parameter models. The phylogram presented here is the majority rule tree. Only those percentage branch credibility values less than 90 are shown (in parentheses). P. trichocarpa GAUT protein sequences are identified by their NCBI RefSeq accessions, except one (designated with *) where the Joint Genome Institute locus identifier was used.

FIG. 2. Transcript Levels of GAUT Genes in WT Arabidopsis Tissues. Semi-quantitative RT-PCR of total RNA isolated from inflorescence (I), silique (S), stem (St), and leaf (L) was used to assess transcript level in Arabidopsis tissues. Gene-specific primers were used to amplify 800 by fragments from the 5′ end of each GAUT open reading frame (Table 1). All reactions were carried out using 2 μg total RNA amplified for 26 PCR cycles. Similar results were obtained in three independent experiments. Control: RT-PCR using primers to L23a small ribosomal protein.

FIG. 3. Glycosyl Residue Composition of Arabidopsis WT Cell Walls. The glycosyl residue composition of walls determined by GC-MS of TMS derivatives was quantified from inflorescence (white bars), silique (light gray bars), stem (dark gray bars), and leaf (black bars) tissues; n≧18. Glycosyl residues are abbreviated as arabinose (Ara), rhamnose (Rha), fucose (Fuc), xylose (Xyl), galacturonic acid (GalA), mannose (Man), galactose (Gal), and glucose (Glc).

FIG. 4. The natural log transformed glycosyl residue composition of GAUT mutant walls. Data are the natural log (ln) transformed normalized mutant wall compositions (±sd) for galacturonic acid (GalA), xylose (Xyl), rhamnose (Rha), galactose (Gal) and arabinose (Ara). A deviation from WT is represented as a departure from 0 on the Y axis, with a positive value for mutant glycosyl residue values greater than WT and a negative value for glycosyl residue values less than WT. GAUT mutants are listed on the X-axis corresponding to GAUT genes with decreasing amino acid similarity to GAUT1 from left to right on the axis. Tissue types: S, silique; L, leaf; I, inflorescence; ST, stem. See Table 3 for description of mutant names (e.g. walls from silique tissue from gaut2-1 is denoted 2-1 S in this Figure).

FIG. 5. Staining and Glycosyl Residue Composition of WT and gaut11-2 Seed Mucilage. Ruthenium red (0.05%) was applied directly to Arabidopsis seeds without shaking. WT seeds (A) clearly show a thick mucilage layer and a dark-staining mucilage envelope that sloughs off of the seed. The gaut11-2 seeds (B, C) extrude less mucilage than similarly treated WT seeds (B) or appear to lack mucilage extrusion almost entirely (C). The gaut11-2 seed mucilage in panel (B) also shows different staining properties from the WT mucilage in panel (A). Inset bar=100 μm. The composition (D) of WT (white bars) and gaut11-2 (gray bars) hot water-extracted mucilage was determined by GC-MS.

FIG. 6. Endogenous expression of GAUT14 transcript in Arabidopsis by qRT-PCR. GAUT14 transcript expression in different plant tissues of Arabidopsis thaliana as measured by qRT-PCR (quantitaive Real Time PCR). RNA prepared from WT plant tissues and cDNA prepared from the RNA was used as a template for qRT-PCR. Amplification of Actin2 was used as a control. Results are the average +/−SD of 3 replicate tissue samples from each of three sets of plants grown at separte times (i.e. N=9).

FIG. 7. Position of T-DNA insertion in Arabidopsis GAUT14 genes. Positions of the T-DNA insertions in GAUT14 gene. Boxes indicate exons, lines indicate introns and open boxes are 5′ and 3′ untranslated regions (UTRs). The T-DNA is inserted in the fourth exon in the gut14-1 line and in the 3′UTR gaut14-2 line.

FIG. 8. Phenotypes and growth measurement of T-DNA gaut14-1 and gaut14-2 knock-out mutants.

FIG. 9. Growth measurement of GAUT14 stem and leaves. Measurement of stem height and length of leaf gaut14 mutants and WT. Each data point is the average of twelve replicates and error bars represents the SD. At each time point, each set of three bars is wild-type (left bar), gaut14-1 (middle bar), and gaut14-2 (right bar).

FIG. 10. Glycome profile of gaut14 leaf in Arabidopsis by ELISA assay

FIG. 11. Glycome profile of gaut14stem in Arabidopsis by ELISA assay.

FIG. 12. Pectin active enzymes in C. bescii (Cbes) and C. saccharolyticus.

FIG. 13. Growth of C. bescii and C. saccharolyticus on Arabidopsis wild type and gaut14 mutants.

FIG. 14. Amino acids and nucleotide sequences of polypeptides and polynucleotides described herein.

### DETAILED DESCRIPTION OF ILLUSTRATIVE EMBODIMENTS

### Polypeptides

The present invention includes, but is not limited to, a transgenic plant having an alteration in expression of a coding region encoding a galacturonosyltransferase (GAUT) polypeptide.

One GAUT polypeptide is referred to herein as GAUT1. Examples of GAUT1 polypeptides are depicted at SEQ ID NO:2 (NP_{—}191672) [Arabodposis], SEQ ID NO:4 (NCBI number EEE81823.1 [Populus]), and SEQ ID NO:6 (NCBI number EEE99060.1 [Populus]).

Another GAUT polypeptide is referred to herein as GAUT2. An example of a GAUT2 polypeptide is depicted at SEQ ID NO:8 (NCBI number NP_{—}182171 [Arabidopsis]).

Another GAUT polypeptide is referred to herein as GAUT3. Examples of GAUT3 polypeptides are depicted at SEQ ID NO:10 (NCBI number NP_{—}195540 [Arabidopsis]), and SEQ ID NO:12 (NCBI number EEE76149.1 [Populus]).

Another GAUT polypeptide is referred to herein as GAUT4. Examples of GAUT4 polypeptides are depicted at SEQ ID NO:14 (NCBI number NP_{—}568688 [Arabidopsis]), SEQ ID NO:16 (NCBI number EEF09095.1 [Populus]), and SEQ ID NO:18 (NCBI number EEE92259.1 [Populus]).

Another GAUT polypeptide is referred to herein as GAUT5/6. Examples of GAUT5/6 polypeptides are depicted at SEQ ID NO: 20 (NCBI number NP_{—}850150 [Arabidopsis]), SEQ ID NO: 22 (NCBI number NP_{—}563771 [Arabidopsis]), and SEQ ID NO:24 (NCBI number EEE94624.1 [Populus]).

Another GAUT polypeptide is referred to herein as GAUT7. Examples of GAUT7 polypeptides are depicted at SEQ ID NO:26 (NCBI number NP_{—}565893 [Arabidopsis]), SEQ ID NO:28 (NCBI number EEE71925.1 [Populus]), and SEQ ID NO:30 (NCBI number EEF05462.1 [Populus]).

Another GAUT polypeptide is referred to herein as GAUT8. Examples of GAUT8 polypeptides are depicted at SEQ ID NO:32 (NCBI number NP_{—}189150 [Arabidopsis]), and SEQ ID NO:34 (NCBI number EEE81076.1 [Populus]).

Another GAUT polypeptide is referred to herein as GAUT9. Examples of GAUT9 polypeptides are depicted at SEQ ID NO:36 (NCBI number NP_{—}566170 [Arabidopsis]), and SEQ ID NO:38 (NCBI number EEF07831.1 [Populus]).

Another GAUT polypeptide is referred to herein as GAUT10. Examples of GAUT10 polypeptides are depicted at SEQ ID NO:40 (NCBI number NP_{—}565485 [Arabidopsis]), SEQ ID NO:42 (NCBI number EEE95846.1 [Populus]), and SEQ ID NO:44 (NCBI number EEF07539.1 [Populus]).

Another GAUT polypeptide is referred to herein as GAUT11. Examples of GAUT11 polypeptides are depicted at SEQ ID NO:46 (NCBI number NP_{—}564057 [Arabidopsis]), SEQ ID NO:48 (NCBI number EEF08400.1 [Populus]), and SEQ ID NO:50 (NCBI number EEE96800.1 [Populus]).

Another GAUT polypeptide is referred to herein as GAUT12. Examples of GAUT12 polypeptides are depicted at SEQ ID NO:52 (NCBI number NP_{—}200280 [Arabidopsis]), SEQ ID NO:54 (NCBI number EEE98176.1 [Populus]), and SEQ ID NO:56 (NCBI number EEE95725.1 [Populus]). Another GAUT polypeptide is referred to herein as GAUT13/14. Examples of GAUT13/14 polypeptides are depicted at SEQ ID NO:58 (NCBI number NP_{—}186753 [Arabidopsis]), SEQ ID NO:60 (NCBI number NP_{—}197051 [Arabidopsis]), SEQ ID NO:62 (NCBI number EEF04227.1 [Populus]), and SEQ ID NO:64 (NCBI number EEE85885.1 [Populus]).

Another GAUT polypeptide is referred to herein as GAUT15. Examples of GAUT15 polypeptides are depicted at SEQ ID NO:66 (NCBI number NP_{—}191438 [Arabidopsis]), and SEQ ID NO:68 (NCBI number EEE99386.1 [Populus]).

Other examples of GAUT polypeptides include those that are structurally similar the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO: 22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, and SEQ ID NO:66. A GAUT polypeptide that is structurally similar to the amino acid sequence of a polypeptide described herein has galacturonosyltransferase activity. Methods for testing whether a polypeptide has galacturonosyltransferase activity are described below.

Structural similarity of two polypeptides can be determined by aligning the residues of the two polypeptides (for example, a candidate polypeptide and any appropriate reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference polypeptide may be a polypeptide described herein. A candidate polypeptide is the polypeptide being compared to the reference polypeptide. A candidate polypeptide may be isolated, for example, from a plant, or can be produced using recombinant techniques, or chemically or enzymatically synthesized. A candidate polypeptide may be inferred from a nucleotide sequence present in the genome of a plant.

Unless modified as otherwise described herein, a pair-wise comparison analysis of amino acid sequences can be carried out using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

Thus, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate polypeptide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

GAUT polypeptides are involved in binding carbohydrates and catalyzing the synthesis of cell wall polysaccharides. GAUT polypeptides are members of the Carbohydrate-Active enZYmes (CAZy) glycosyltransferase family 8 (GT8) (Yin et al., 2010, Plant Physiol., 153:1729-1.746). The CAZy database describes the families of structurally-related catalytic and carbohydrate-binding modules (or functional domains) of enzymes that degrade, modify, or create glycosidic bonds (Cantarel et al., 2009, Nucleic Acids Res., 37:D233-238; Campbell et al., 1997, Biochem. J. 326:929-939; Coutinho et al., 2003, J. Mol. Biol. 328:307-317).

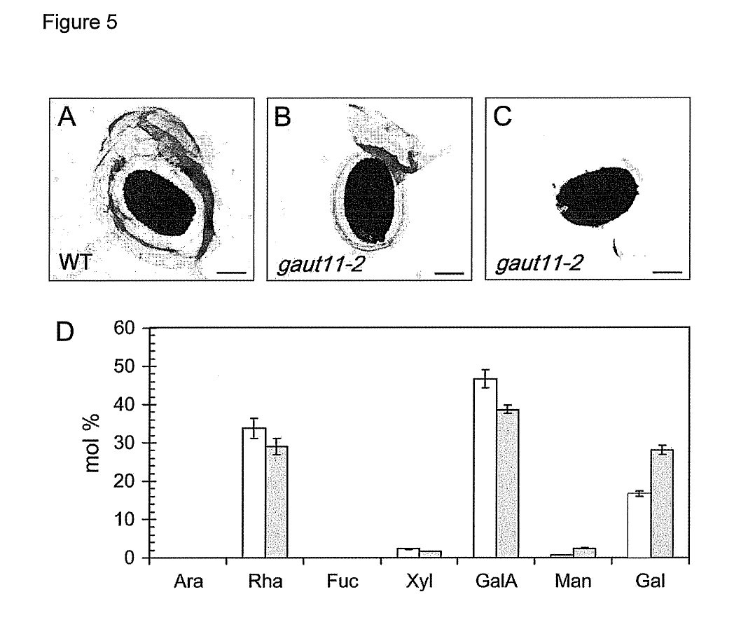

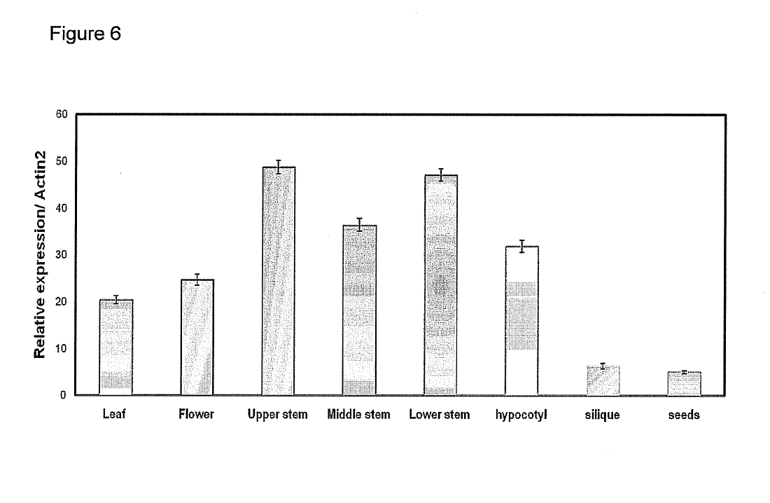

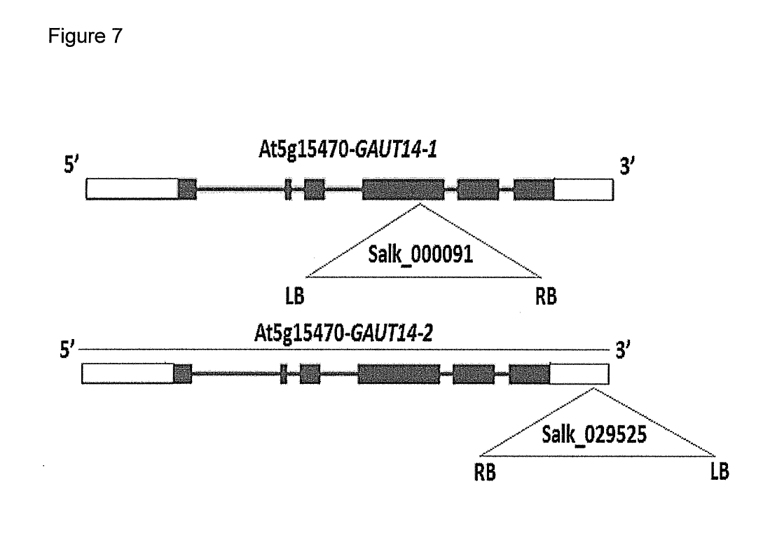

The GAUT polypeptides contain several conserved domains involved in substrate binding and catalysis. Conserved amino acid sequences are described by Yin et al. (2010, Plant Physiol., 153:1729-1746, including FIG. 5 therein) and include the putative catalytic site HXXGXXKPW (where X refers to any amino acid), DXDXVVQXD, WHXXXXXGLGY, LPXXLXXF, CXWXXXMNXXDXXXW, and RFYXPEXXP.

A GAUT polypeptide has galacturonosyltransferase activity. Whether a polypeptide has galacturonosyltransferase activity can be determined by producing a transgenic plant that has decreased expression of a candidate polypeptide and observing the phenotype of the transgenic plant. A transgenic plant deficient in the expression of one or more GAUT polypeptides may display one or more useful phenotypes as described herein. In one embodiment, decreased expression of a polypeptide having galacturonosyltransferase activity in a transgenic plant results in decreased recalcitrance. In one embodiment, decreased expression of a polypeptide having galacturonosyltransferase activity in a transgenic plant results in a plant with increased growth, such as increased height and/or increased diameter.

### Polynucleotides

Examples of polynucleotides encoding SEQ ID NO:2, SEQ ID NO:8, SEQ ID NO:10, SEQ ID NO:14, SEQ ID NO: 20, SEQ ID NO: 22, SEQ ID NO:26, SEQ ID NO:32, SEQ ID NO:36, SEQ ID NO:40, SEQ ID NO:46, SEQ ID NO:52, SEQ ID NO:58, SEQ ID NO:60, and SEQ ID NO:66 are shown at SEQ ID NO:1, SEQ ID NO: 7, SEQ ID NO: 9, SEQ ID NO:13, SEQ ID NO: 19, SEQ ID NO: 21, SEQ ID NO:25, SEQ ID NO:31, SEQ ID NO:35, SEQ ID NO:39, SEQ ID NO:45, SEQ ID NO:51, SEQ ID NO:57, SEQ ID NO:59, and SEQ ID NO:65, respectively. It should be understood that a polynucleotide encoding one of the GAUT polypeptides is not limited to a nucleotide sequence disclosed herein, but also includes the class of polynucleotides encoding the GAUT polypeptides as a result of the degeneracy of the genetic code. For example, the naturally occurring nucleotide sequence SEQ ID NO:1 is but one member of the class of nucleotide sequences encoding a polypeptide having the amino acid sequence SEQ ID NO:2. The class of nucleotide sequences encoding a selected polypeptide sequence is large but fmite, and the nucleotide sequence of each member of the class may be readily determined by one skilled in the art by reference to the standard genetic code, wherein different nucleotide triplets (codons) are known to encode the same amino acid.

While the polynucleotide sequences described herein are listed as DNA sequences, it is understood that the complements, reverse sequences, and reverse complements of the DNA sequences can be easily determined by the skilled person.

It is also understood that the sequences disclosed herein as DNA sequences can be converted from a DNA sequence to an RNA sequence by replacing each thymidine nucleotide with a uracil nucleotide.

Structural similarity of two polynucleotides can be determined by aligning the residues of the two polynucleotides (for example, a candidate polynucleotide and any appropriate reference polynucleotide described herein) to optimize the number of identical amino acids along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. A candidate polynucleotide is the polynucleotide being compared to the reference polynucleotide. A candidate polynucleotide may be isolated, for example, from a plant, or can be produced using recombinant techniques, or chemically or enzymatically synthesized. A candidate polynucleotide may be present in the genome of a plant and predicted to encode a GAUT polypeptide.

Unless modified as otherwise described herein, a pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence.

The present invention also provides methods of using GAUT polypeptides and polynucleotides encoding GAUT polypeptides. The present invention includes methods for altering expression of plant. GAUT coding regions for purposes including, but not limited to (i) investigating function of biosynthesis of pectin and ultimate effect on plant phenotype, (ii) effecting a change in plant phenotype, and (iii) using plants having an altered phenotype.

The present invention includes methods for altering the expression of any of the coding regions encoding the GAUT polypeptides disclosed herein. Thus, for example, the invention includes altering expression of a GAUT coding region present in the genome of a wild-type plant. As disclosed herein, in one embodiment a wild-type plant is a woody plant, such as a member of the species Populus.

Techniques which can be used in accordance with the present invention to alter expression of a GAUT coding region, include, but are not limited to: (i) disrupting a coding region's transcript, such as disrupting a coding region's mRNA transcript; (ii) disrupting the function of a polypeptide encoded by a coding region, (iii) disrupting the coding region itself, (iv) modifying the timing of expression of the coding region by placing it under the control of a non-native promoter, or (v) over-expression the coding region. The use of antisense RNAs, ribozymes, double-stranded RNA interference (dsRNAi), and gene knockouts are valuable techniques for discovering the functional effects of a coding region and for generating plants with a phenotype that is different from a wild-type plant of the same species.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726; Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving (degrading) the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Small, 2007, Curr. Opin. Biotechnol., 18:148-153; McGinnis, 1010, Brief. Funct. Genomics, 9(2): 111-117.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the polypeptide encoded by the coding region. For instance, mutations in a coding region and/or an operably linked regulatory region may be made by deleting, substituting, or adding a nucleotide(s).The use of T-DNA based inactiviation is discussed, for example, in Azpiroz-Leehan et al. (1997, Trends in Genetics, 13:152-156).

Over-expression of a coding region may be accomplished by cloning the coding region into an expression vector and introducing the vector into recipient cells. Alternatively, over-expression can be accomplished by introducing exogenous promoters into cells to drive expression of coding regions residing in the genome. The effect of over-expression of a given coding region on the phenotype of a plant can be evaluated by comparing plants over-expressing the coding region to control plants.

Altering expression of a GAUT coding region may be accomplished by using a portion of a polynucleotide described herein. In one embodiment, a polynucleotide for altering expression of a GAUT coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of a GAUT coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of a GAUT coding region in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a GAUT coding region in a plant cell includes substantially all of a coding region, or in some cases, an entire coding region. An antisense strand is substantially complementary, preferably, complementary, to a target coding region or a target mRNA. As used herein, the term “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode an antisense RNA molecule or a molecule useful in RNAi. Promoters useful in the invention include constitutive promoters, inducible promoters, and/or tissue preferred promoters for expression of a polynucleotide in a particular tissue or intracellular environment, examples of which are known to one of ordinary skill in the art.

Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

Another example of a regulatory region is a transcription terminator. Suitable transcription terminators are known in the art and include, for instance, a stretch of 5 consecutive thymidine nucleotides.

Thus, in one embodiment a polynucleotide that is operably linked to a regulatory sequence may be in an “antisense” orientation, the transcription of which produces a polynucleotide which can foim secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli. Preferably the vector is a plasmid. In some embodiments, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli.

A selection marker is useful in identifying and selecting transformed plant cells or plants. Examples of such markers include, but are not limited to, a neomycin phosphotransferase (nptII) gene (Potrykus et al., 1985, Mol. Gen. Genet., 199:183-188), which confers kanamycin resistance. Cells expressing the nptll gene can be selected using an appropriate antibiotic such as kanamycin or G418. Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide of the present invention in a cell, and the polynucleotide may then be isolated from the cell.

### Host Cells, Plants, and Transgenic Plants

The invention also provides host cells having altered expression of a coding region described herein. As used herein, a host cell includes the cell into which a polynucleotide described herein was introduced, and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

The present invention further provides transgenic plants having altered expression of a coding region. A transgenic plant may be homozygous or heterozygous for a modification that results in altered expression of a coding region.

The present invention also includes natural variants of plants, where the natural variants have increased or decreased expression of GAUT polypeptides. In one embodiment, GAUT expression is decreased. The change in GAUT expression is relative to the level of expression of the GAUT polypeptide in a natural population of the same species of plant. Natural populations include natural variants, and at a low level, extreme variants (Studer et al., 2011, 108:6300-6305). The level of expression of GAUT polypeptide in an extreme variant may vary from the average level of expression of the GAUT polypeptide in a natural population by at least 5%, at least 10%, at least 15%, at least 20%, or at least 25%. The average level of expression of the GAUT polypeptide in a natural population may be determined by using at least 50 randomly chosen plants of the same species as the putative extreme variant.

The plants may be angiosperms or gymnosperms. The polynucleotides described herein may be used to transform a variety of plants, both monocotyledonous (e.g grasses, corn, grains, oat, wheat, barley), dicotyledonous (e.g., Arabidopsis, tobacco, legumes, alfalfa, oaks, eucalyptus, maple, poplar, aspen, cottonwood), and Gymnosperms (e.g., Scots pine, white spruce, and larch).

The plants also include switchgrass, turfgrass, wheat, maize, rice, sugar beet, potato, tomato, lettuce, carrot, strawberry, cassava, sweet potato, geranium, soybean, and various types of woody plants. Woody plants include trees such as palm oak, pine, maple, fir, apple, fig, plum acacia, poplar, aspen, cottonwood, and willow. Woody plants also include rose and grape vines.

In one embodiment, the plants are woody plants, which are trees or shrubs whose stems live for a number of years and increase in diameter each year by the addition of woody tissue. The invention plants of significance in the commercial biomass industry such as members of the family Salicaceae, such as Populus spp. (e.g., Populus trichocarpa, Populus deltoides), pine, and Eucalyptus spp. Also included in the present invention is the wood and wood pulp derived from the plants described herein.

Transformation of a plant with a polynucleotide described herein may yield a phenotype including, but not limited to any one or more of changes in height, yield, lignin quality, lignin structure, amount of lignin, pectin structure, hemicellulose structure, glycoconjugate structure, wood composition, wood strength, cellulose polymerization, fiber dimensions, cell wall composition (such as cell wall polysaccharide content), rate of wood formation, rate of growth, increased infloresence, and leaf shape. In one embodiment a phenotype is increased height compared to a control plant. In one embodiment a phenotype is reduced recalcitrance compared to a control plant. Methods for measuring recalcitrance are routine and include, but are not limited to, measuring changes in the extractability of carbohydrates, where an increase in extractability suggests a more loosely held together wall, and thus, decreased recalcitrance. Another test for measuring changes in recalcitrance use microbes and is described below. In one embodiment a phenotype is reduced lignin compared to a control plant. Methods for measuring lignin are routine and include, but are not limited to, staining cells with phoroglucinol. A decrease in ligninfication can result in decreased recalcitrance.

Other phenotypes present in a transgenic plant described herein may include yielding biomass with reduced recalcitrance and from which sugars can be released more efficiently for use in biofuel and biomaterial production, yielding biomass which is more easily deconstructed and allows more efficient use of wall structural polymers and components, and yielding biomass that will be less costly to refine for recovery of sugars and biomaterials.

Phenotype can be assessed by any suitable means. The plants may be evaluated based on their general morphology. Transgenic plants can be observed with the naked eye, can be weighed and their height measured. The plant can be examined by isolating individual layers of plant tissue, namely phloem and cambium, which is further sectioned into meristematic cells, early expansion, late expansion, secondary wall formation, and late cell maturation. The plants also can be assessed using microscopic analysis or chemical analysis.

Microscopic analysis includes examining cell types, stage of development, and stain uptake by tissues and cells. Fiber morphology, such as fiber wall thickness may be observed using, for example, microscopic transmission ellipsometry (Ye and Sundstrom, 1977, Tappi J., 80:181). Wood strength and density in wet wood and standing trees can be determined by measuring the visible and near infrared spectral data in conjunction with multivariate analysis (Gabor, U.S. Pat. No. 6,525,319). Lumen size can be measured using scanning electron microscopy. Lignin structure and chemical properties, (such as cell wall properties) can be observed using nuclear magnetic resonance spectroscopy, chemical derivatization, mass spectrometry, diverse microscopies, colorimetric assays, glycome profiling.

The biochemical characteristic of lignin, cellulose, carbohydrates and other plant extracts can be evaluated by standard analytical methods including spectrophotometry, fluorescence spectroscopy, HPLC, mass spectroscopy, molecular beam mass spectroscopy, near infrared spectroscopy, nuclear magnetic resonance spectroscopy, and tissue staining methods.

One method that can be used to evaluate the phenotype of a transgenic plant is glycome profiling. Glycome profiling gives information about the presence of carbohydrate structures in plant cell walls, including changes in the extractability of carbohydrates from cell walls (Zhu et al., 2010, Mol. Plant, 3:818-833; Pattathil et al., 2010, Plant Physiol., 153:514-525), the latter providing information about larger scale changes in wall structure. Diverse plant glycan-directed monoclonal antibodies are available from, for instance, CarboSource Services (Athens, Ga.), and PlantProbes (Leeds, UK).

In one embodiment, a transgenic plant has changes in carbohydrates of the homogalacturonan (HG) backbone, changes in carbohydrates of the rhamnogalacturonan-1 backbone, changes in rhamnogalacturonan-1/arabinogalactan (AG), changes in xylan-2, changes in xylan-3, changes in xylan-4, changes in rhamnogalacturonan-1b changes in rhamnogalactmonan-1c, changes in AG-1, changes in AG-2, changes in AG-3, changes in AG-4, changes in non-fucosylated xyloglucan (NON-FUC XG), changes in galactomannan, changes in AG-3, or a combination thereof. The change may be an increase or a decrease of one or more of these carbohydrates in an extracted fraction compared to a control plant. In one embodiment the change is an increase of one or more of these carbohydrates in an extracted fraction compared to a control plant. Examples of solvents useful for evaluating the extractability of carbohydrates include, but are not limited to, oxalate, carbonate, KOH (e.g., 1M and 4M), and chlorite.

### Methods for Measuring Changes in Recalcitrance

Provided herein are methods for testing recalcitrance of plant biomass. The method uses microbial strains that are known to be deficient in the ability to grow on (e.g., degrade) a particular constituent of plant biomass. For instance, in one embodiment, the microbial strain Caldicellulosiruptor saccharolyticus may be used, as it is deficient in the ability to degrade structures present in pectin. When C. saccharolyticus is used, an appropriate control is C. bescii, a strain that is not deficient is the ability to degrade pectin when compared to C. saccharolyticus. C. saccharolyticus and C. bescii are available from the Deutsche Sammlung von Mikroorganismen and Zellkulturen GmbH (DSM) as strain numbers 8903 and 6725, respectively. Such an assay can be useful in comparing a transgenic plant and a control plant.

In general, the method includes growing under suitable conditions two cultures of a microbe that is deficient in the ability to degrade a constituent of plant biomass. One culture includes material obtained from a first plant, and the second culture includes material obtained from a second plant. Any material from a plant may be used, such as stem, leaves, etc. The material may be processed (pretreated) as described below. The first plant may be a transgenic plant described herein and the second plant may be a control plant. After a suitable time for replication, the growth characteristics of the microbe in the two cultures are compared. Suitable growth characteristics may include time to reach stationary phase and final cell density. A microbe that reaches stationary phase more quickly or has a greater cell density after growth in the presence of transgenic plant material when compared to the microbe grown in the presence of control plant material indicates the transgenic plant has some alteration in a constituent of plant biomass. The alteration may be a decreased amount of the constituent in the transgenic plant, or that the constituent is modified in the transgenic plant.

In one embodiment, the method includes growing under suitable conditions two cultures of C. saccharolyticus. One culture includes material obtained from a first plant, and the second culture includes material obtained from a second plant. The first plant may be a transgenic plant described herein and the second plant may be a control plant. After a suitable time for replication of the C. saccharolyticus the growth characteristics of the microbe in the two cultures is compared. If the C. saccharolyticus grown on the transgenic plant reaches stationary phase in a shorter time or achieves a higher cell density when compared to the control cell, then the assay suggests that the transgenic plant has a decreased amount of pectin or that the pectin is modified in the transgenic plant, and that the transgenic plant has reduced recalcitrance compared to the control plant.

Another method for measuring recalcitrance involves treated non-pretreated, or heat or chemical pretreated plant biomass with a specific set of enzymes, which may include one or more cellulases or hemicellulases, e.g., enzymes that degrade cellulose and hemicelluloses, respectively. The biomass may also be treated with additional enzymes that include, but are not limited to pectinases. Following treatment the material released from the non-soluble biomass is measured, for example, for reducing sugars or for specific glycosyl residue composition using standard methods (Studer et al, 2011, Proc. Natl. Acad. Sci., U.S.A., 108:6300-6305). The biomass that provides a greater amount of released sugar under identical pretreatment and enzyme treatment conditions is said to have reduced recalcitrance, i.e. is more easily deconstructed.

### Methods for Making

Transgenic plants described herein may be produced using routine methods. Methods for transformation and regeneration are known to the skilled person. Transformation of a plant cell with a polynucleotide described herein may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

Transformation techniques for dicotyledons are known in the art and include Agrobacterium-based techniques and techniques that do not require Agrobacterium. Non Agrobacterium techniques involve the uptake of exogenous genetic material directly by protoplasts or cells. This may be accomplished by PEG or electroporation mediated-uptake, particle bombardment-mediated delivery, or microinjection. In each case the transformed cells may be regenerated to whole plants using standard techniques known in the art.

Techniques for the transformation of monocotyledon species include direct gene transfer into protoplasts using PEG or electroporation techniques, particle bombardment into callus tissue or organized structures, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

### Methods of Use

Provided herein are methods for using the plants described herein. In one embodiment, the methods include producing a metabolic product. A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus the invention includes methods for processing a transgenic plant to result in a pulp, methods for hydrolyzing a pulp that contain cells from a transgenic plant, and methods for producing a metabolic product from a pulp.

There are numerous methods or combinations of methods known in the art and routinely used to process plants. The result of processing a plant is a pulp. As used herein, “pulp” refers to processed plant material. Plant material, which can be any part of a plant, may be processed by any means, including mechanical, chemical, biological, or a combination thereof Mechanical pretreatment breaks down the size of plant material. Biomass from agricultural residues is often mechanically broken up during harvesting. Other types of mechanical processing include milling or aqueous/steam processing. Chipping or grinding may be used to typically produce particles between 0.2 and 30 mm in size. Methods used for plant materials may include intense physical pretreatments such as steam explosion and other such treatments (Peterson et al., U.S. Patent Application 20090093028). The most common chemical pretreatment methods used for plant materials include dilute acid, alkaline, organic solvent, ammonia, sulfur dioxide, carbon dioxide or other chemicals to make the biomass more available to enzymes. Biological pretreatments are sometimes used in combination with chemical treatments to solubilize lignin in order to make cellulose more accessible to hydrolysis and fermentation. In one embodiment, a method for using transgenic plants described herein includes processing plant material to result in a pulp. In one embodiment, transgenic plants described herein, such as those with reduced recalcitrance and/or decreased lignification, are expected to require less processing than a control plant. The conditions described below for different types of processing are for a control plant, and the use of a plant as described herein is expected to require less severe conditions.

Steam explosion is a common method for pretreatment of plant biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes degradation of cell wall complex carbohydrates and lignin transformation. Addition of H₂SO₄, SO₂, or CO₂ to the steam explosion reaction can improve subsequent cellulose hydrolysis (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of plant biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment. Hot water, for example 140° C. or 160° C. or 180° C. can also be used as a pretreatment of plant biomass (Studer et al, 2011, Proc. Natl. Acad. Sci., U.S.A., 108:6300-6305).

Methods for hydrolyzing a pulp may include enzymatic hydrolysis. Enzymatic hydrolysis of processed biomass includes the use of cellulases. Some of the pretreatment processes described above include hydrolysis of complex carbohydrates, such as hemicellulose and cellulose, to monomer sugars. Others, such as organosolv, prepare the substrates so that they will be susceptible to hydrolysis. This hydrolysis step can in fact be part of the fermentation process if some methods, such as simultaneous saccharification and fermentation (SSF), are used. Otherwise, the pretreatment may be followed by enzymatic hydrolysis with cellulases.

A cellulase may be any enzyme involved in the degradation of the complex carbohydrates in plant cell walls to fermentable sugars, such as glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, e.g., hydrolyze cellulose, either in the acid, neutral, or alkaline pH-range.

A cellulase may be of fungal or bacterial origin, which may be obtainable or isolated from microorganisms which are known to be capable of producing cellulolytic enzymes. Useful cellulases may be produced by fermentation of the above-noted microbial strains on a nutrient medium containing suitable carbon and nitrogen sources and inorganic salts, using procedures known in the art.

Examples of cellulases suitable for use in the present invention include, but are not liminted to, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH).

The hydrolysis/fermentation of plant material may, and typically does, require addition of cellulases (e.g., cellulases available from Novozymes A/S). Typically, cellulase enzymes may be added in amounts effective from 5 to 35 filter paper units of activity per gram of substrate, or, for instance, 0.001% to 5.0% wt. of solids. The amount of cellulases appropriate for the hydrolysis may be decreased by using a transgenic plant described herein. The amount of cellulases (e.g., cellulases available from Novozymes A/S) required for hydrolysis of the pretreated plant biomass may be decreased by at least 5%, at least 10%, at least 15%, at least 20%, at least 25%, or at least 30% compared to the amount of cellulases required for hydrolysis of a control plant. This decreased need for cellulases can result in a significant decrease in costs associated with producing metabolic products from plant materials.

The steps following pretreatment, e.g., hydrolysis and fermentation, can be performed separately or simultaneously. Conventional methods used to process the plant material in accordance with the methods disclosed herein are well understood to those skilled in the art. Detailed discussion of methods and protocols for the production of ethanol from biomass are reviewed in Wyman (1999, Annu. Rev. Energy Environ., 24:189-226), Gong et al. (1999, Adv. Biochem. Engng. Biotech., 65: 207-241), Sun and Cheng (2002, Bioresource Technol., 83:1-11), and Olsson and Hahn-Hagerdal (1996, Enzyme and Microb. Technol., 18:312-331). The methods of the present invention may be implemented using any conventional biomass processing apparatus (also referred to herein as a bioreactor) configured to operate in accordance with the invention. Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Appl. Biochem. Biotechnol., 56: 141-153). Smaller scale fermentations may be conducted using, for instance, a flask.

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HIS), and direct microbial conversion (DMC). The fermentation can be carried out by batch fermentation or by fed-batch fermentation.

SHF uses separate process steps to first enzymatically hydrolyze plant material to glucose and then ferment glucose to ethanol. In SSF, the enzymatic hydrolysis of plant material and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the coferementation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

The final step may be recovery of the metabolic product. Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

Transgenic plants described herein may also be used as a feedstock for livestock. Plants with reduced recalcitrance are expected to be more easily digested by an animal and more efficiently converted into animal mass. Accordingly, the present invention includes methods for using a transgenic plant as a source for a feedstock, and includes a feedstock that has plant material from a transgenic plant as one of its components.

The present invention is illustrated by the following examples. It is to be understood that the particular examples, materials, amounts, and procedures are to be interpreted broadly in accordance with the scope and spirit of the invention as set forth herein.

### Example 1

### Methods

Sequence Alignment of GAUT Family Proteins and Phylogenetic Analysis

Protein sequences were identified by BLASTsearch of Arabidopsis thaliana (www.Arabidopsis.org/index.jsp), Oryza sativa (www.tigr.org/tdb/e2k1/osa1/), and Populus trichocarpa (http://genome.jgi-psf.org/Poptr1_{—}1/Poptr1_{—}1.home.html) genomes, using AtGAUT1 as the search probe. The GAUT protein sequences were aligned using ClustalX (Thompson et al., 1997, Nucleic Acids Res. 24, 4876-4882) and suggested protein alignment parameters (Hall, B. G. 2004, Phylogenetic Trees Made Easy: A How-To Manual, 2nd ed, (Sunderland, M A: Sinauer Associates, Inc.), pp 29-30). Phylogenetic Bayesian analysis was carried out employing MrBayes (Huelsenbeck and Ronquist, 2001, Bioinformatics. 17, 754-755; Ronquist and Huelsenbeck, 2003, Bioinformatics, 19, 1574). Full-length protein sequences were used in the analysis for all proteins except Os09g36180, whose C-terminal 404 amino acid extension was excluded.

Plant Materials and Growth Conditions

Arabidopsis thaliana var. Columbia S6000 T-DNA insertion mutant seeds were obtained from the Arabidopsis Biological Resource Center (www.biosci.ohio-state.edu/pcmb/Facilities/abrc/abrchome.htm). Arabidopsis WT and gaut mutant seeds were sown on pre-moistened soil and grown to maturity under 60% constant relative humidity with a 14/10 light/dark cycle (14 h (19° C.; 150 microEi m⁻² s⁻¹)/10 h (15° C.)). The plants were fertilized (Peters 20/20/20 with micronutrients) once a week or as needed. WT and T-DNA insert mutant seeds were sown in ‘growth sets’ of 20 plants. Walls were harvested from multiple 8-week-oldWT and PCR-genotyped mutant plants and pooled, respectively, together for wall glycosyl residue composition analysis. The following tissues were harvested for the wall analyses: the apical inflorescence excluding the young siliques; the young fully expanded leaves approximately 3 cm long; green siliques; and the top 8 cm of actively growing stem minus the inflorescence and siliques.

DNA Extraction and Mutant Genotyping

Fresh, flash-frozen leaf tissue (100-200 mg) was ground with a mortar and pestle and suspended in 0.5 ml extraction buffer (100 mM Tris-HCl pH 8.0, 100 mM EDTA pH 8.0, 250 mM NaCl, 100 lg ml⁻⁴ proteinase K and 1% (w/v) n-lauroylsarcosine) and extracted with an equal volume of phenol:chloroform:isoamyl alcohol (49:50:1, v/v). RNA was degraded by addition of 2 microliter of DNase-free RNase A (10 mg ml⁻¹) for 20 min at 37° C. The DNA was precipitated twice with 70% (v/v) ethanol and suspended in a final volume of 50 microliter. Primers used for mutant genotyping were designed by ISECT tools (http://signal.salk.edu/isects.html). The genotype of mutant plants was determined based on the ability of the LB primers to anneal and produce T-DNA-specific PCR products when combined with the appropriate GAUT gene-specific primer. Gene-specific primer pairs were similarly used to determine the presence of intact GAUT genes (see Table 1).

TABLE 1
Primer sequences used in the GAUT analyses.

| | | Primer | 5′ to 3′ primer sequence | |
|---|---|---|---|---|
| Locus | GAUT | name | (SEQ ID NO: 69-159) | Notes |
| At3g61130 | 1 | gs | ATG GCG CTA AAG CGA GGG CTA TCT | For RT- |
| | | At1g61130 | GGA (69) | PCR |
| | | F | | |
| At3g61130 | 1 | gs | TCG TTC TTG TTT TTC AAT TTT GCA ATC | For RT- |
| | | At1g61130 | (70) | PCR |
| | | R | | |
| At2g46480 | 2 | gs | ATG ACT GAT GCT TGT TGT TTG AAG | For RT- |
| | | At2g46480 | GGA | PCR |
| | | F | | |
| At2g46480 | 2 | gs | ATC AGA GAA GAG AGC GTA GTG GTA | For RT- |
| | | At2g46480 | AAG | PCR |
| | | R | | |
| At4g38270 | 3 | gs | ATG TCG GTG GAG CCA TTT TAG AGT | For RT- |
| | | At4g38270 | CAC | PCR |
| | | F | | |
| At4g38270 | 3 | gs | TTG AAG GAA GGT CAG CAT CAG AGG | For RT- |
| | | At4g38270 | TTG | PCR |
| | | R | | |
| At5g47780 | 4 | gs | ATG ATG GTG AAG CTT CGC AAT CTT | For RT- |
| | | At5g47780 | GTT | PCR |
| | | F | | |
| At5g47780 | 4 | gs | GGA GCA TAG CAC GTA GCT TCT TGA | For RT- |
| | | At5g47780 | CCA | PCR |
| | | R | | |
| At2g30575 | 5 | gs | ATG AAT CAA GTT CGT CGT TGG CAG | For RT- |
| | | At2g30575 | AGG | PCR |
| | | F | | |
| At2g30575 | 5 | gs | TGT GAA AGG CAC GGC TGA CCT TGT | For RT- |
| | | At2g30575 | ATA | PCR |
| | | R | | |
| At1g06780 | 6 | gs | ATG AAA CAA ATT CGT CGA TGG CAG | For RT- |
| | | At1g06780 | AGG | PCR |
| | | F | | |
| At1g06780 | 6 | gs | CTT CTG TGT TAT AAT TCA TGG CAC | For RT- |
| | | At1g06780 | GGA | PCR |
| | | R | | |
| At2g38650 | 7 | gs | ATG AAA GGC GGA GGC GGT GGT GGA | For RT- |
| | | At2g38650 | GGA | PCR |
| | | F | | |
| At2g38650 | 7 | gs | CTT CAC AAG TTC TCC AAG TTT CAT | For RT- |
| | | At2g38650 | CAC CA | PCR |
| | | R | | |
| At3g25140 | 8 | gs | ATG GCT AAT CAC CAC CGA CTT TTA | For RT- |
| | | At3g25140 | CGC | PCR |
| | | F | | |
| At3g25140 | 8 | gs | GTA AAG ATT CGG ATC CTC GAG CTC CC | For RT- |
| | | At3g25140 | G | PCR |
| | | R | | |
| At3g02350 | 9 | gs | ATG GGC AAC GCA TAT ATG CAG AGG | For RT- |
| | | At3g02350 | ACG | PCR |
| | | F | | |
| At3g02350 | 9 | gs | CAC CTT CAT GGC TGC GAG ATT CAT | For RT- |
| | | At3g02350 | CCG | PCR |
| | | R | | |
| At2g20810 | 10 | gs | ATG AGA AGG AGA GGA GGG GAT AGT | For RT- |
| | | At2g20810 | TTC | PCR |
| | | F | | |
| At2g20810 | 10 | gs | CCA CAA CAG AAG TAG CAA TAA TGT | For RT- |
| | | At2g20810 | TAT | PCR |
| | | R | | |
| At1g18580 | 11 | gs | ATG AGG CGG TGG CCG GTG GAT CAC | For RT- |
| | | At1g18580 | CGG | PCR |
| | | F | | |
| At1g18580 | 11 | gs | CTC ATC TGC CAG TTC ATG GCG AGA | For RT- |
| | | At1g18580 | TGG | PCR |
| | | R | | |
| At5g54690 | 12 | gs | ATG CAG TTA CAT ATA TCT CCG AGC | For RT- |
| | | At5g54690 | TTG | PCR |
| | | F | | |
| At5g54690 | 12 | gs | TAG CCA CAA CCG AAG CTG CAA GAA | For RT- |
| | | At5g54690 | TAT | PCR |
| | | R | | |
| At3g01040 | 13 | gs | ATG CAG CTT CAC ATA TCG CCT AGC | For RT- |
| | | At3g01040 | ATG | PCR |
| | | F | | |
| At3g01040 | 13 | gs | TTC TTG TCT GTG ATA ACA TGG AAG | For RT- |
| | | At3g01040 | ACA | PCR |
| | | R | | |
| At5g15470 | 14 | gs | ATG CAG CTT CAC ATA TCG CCT AGC | For RT- |
| | | At5g15470 | ATG | PCR |
| | | F | | |
| At5g15470 | 14 | gs | CAG CAG ATG AGA CCA CAA CCG ATG | For RT- |
| | | At5g15470 | CAG | PCR |
| | | R | | |
| At3g58790 | 15 | gs | ATG AAG TTT TAC ATA TCA GCG ACG | For RT- |
| | | At3g58790 | GGG AT | PCR |
| | | F | | |
| At3g58790 | 15 | gs | CGA GCC ATT GCA TTT ACA GAG TAC | For RT- |
| | | At3g58790 | TCT TC | PCR |
| | | R | | |
| | | L23alpha F | CCA TGT CTC CGG CTA AAG TTG ATA C | For RT- |
| | | | | PCR |
| | | L23alpha R | CAG CAC GAA TGT CAA CAA TGA AAA | For RT- |
| | | | CA | PCR |
| At2g46480 | 2 | 122209 F | tcagaagaagtttgaactgagttagccac | iSECT |
| | | | | tools T- |
| | | | | DNA |
| | | | | insertion |
| | | | | site |
| At2g46480 | 2 | 122209 R | atgtttaacaagcccaataaggcataatc | iSECT |
| | | | | tools T- |
| | | | | DNA |
| | | | | insertion |
| | | | | site |
| At4g38270 | 3 | 001920 F | TTTGAAAACTCAGTCATAGGGAAATA | iSECT |
| | | | | tools T- |
| | | | | DNA |
| | | | | insertion |
| | | | | site |
| At4g38270 | 3 | 001920 R | GAAGGATGATTTGCTTTGAAATAGTA | iSECT |
| | | | | tools T- |
| | | | | DNA |
| | | | | insertion |
| | | | | site |
| At4g38270 | 3 | 113167 F | Accaggttaaagccattgtagagtgaaat | iSECT |
| | | | | tools T- |
| | | | | DNA |
| | | | | insertion |
| | | | | site |
| At4g38270 | 3 | 113167 R | atgtagcactactacctgcaaatcgtc | iSECT |
| | | | | tools T- |
| | | | | DNA |
| | | | | insertion |
| | | | | site |
| At2g30575 | 5 | 050186 F | GATCATTATAACTTTGTTGCAAAAGCTGC | iSECT |
| | | | | tools T- |
| | | | | DNA |
| | | | | insertion |
| | | | | site |
| At2g30575 | 5 | 050186 R | AATGCGGAGGTACGTAGTTTAATCCAGTT | iSECT |
| | | | | tools T- |
| | | | | DNA |
| | | | | insertion |
| | | | | site |
| At2g30575 | 5 | 058223 F | taatgttgagatacagatatagtgcggcg | iSECT |
| | | | | tools T- |
| | | | | DNA |
| | | | | insertion |
| | | | | site |
| At2g30575 | 5 | 058223 R | aaaattcaaagctagctgaagtaaaagtg | iSECT |
| | | | | tools T- |
| | | | | DNA |
| | | | | insertion |
| | | | | site |
| At1g06780 | 6 | 007987 F | ttatctaagggtgaaaagaacacaagggt | iSECT |
| | | | | tools T- |
| | | | | DNA |
| | | | | insertion |
| | | | | site |
| At1g06780 | 6 | 007987 R | acattgagattgctgggtaattaagtgaa | iSECT |
| | | | | tools T- |
| | | | | DNA |
| | | | | insertion |
| | | | | site |
| At1g06780 | 6 | 056646 F | cagggaagaacaagtgattgtttca | iSECT |
| | | | | tools T- |
| | | | | DNA |
| | | | | insertion |
| | | | | site |
| At1g06780 | 6 | 056646 R | gaaatgcatgatacctttgatgaaga | iSECT |
| | | | | tools T- |
| | | | | DNA |
| | | | | insertion |
| | | | | site |
| At1g06780 | 6 | 073484 F | catagtcaacgttaacacccatttgactt | iSECT |
| | | | | tools T- |
| | | | | DNA |
| | | | | insertion |
| | | | | site |
| At1g06780 | 6 | 073484 R | ctcttaagccgattcgatacgaaaataag | iSECT |
| | | | | tools T- |
| | | | | DNA |
| | | | | insertion |
| | | | | site |
| At2g38650 | 7 | 015189 F | atatcaaggtcccaaaggggagataagt | iSECT |
| | | | | tools T- |
| | | | | DNA |
| | | | | insertion |
| | | | | site |
| At2g38650 | 7 | 015189 R | ctcaagagaagctttgatgtgtagaatcc | iSECT |
| | | | | tools T- |
| | | | | DNA |
| | | | | insertion |
| | | | | site |
| At2g38650 | 7 | 046348 F | ttcggatacatctctctgcaaaacc | iSECT |
| | | | | tools T- |
| | | | | DNA |
| | | | | insertion |
| | | | | site |
| At2g38650 | 7 | 046348 R | cttgcaccagattgaacctaaatgg | iSECT |
| | | | | tools T- |
| | | | | DNA |
| | | | | insertion |
| | | | | site |
| At3g25140 | 8 | 030075 F | gatcaaagagaagtttaatcccaaagcat | iSECT |
| | | | | tools T- |
| | | | | DNA |
| | | | | insertion |
| | | | | site |
| At3g25140 | 8 | 030075 R | taattggagtcaaaacttgagagcaagag | iSECT |
| | | | | tools T- |
| | | | | DNA |
| | | | | insertion |
| | | | | site |
| At3g25140 | 8 | 102380 F | tctcttctaatgatctaatcccacaataa | iSECT |
| | | | | tools T- |
| | | | | DNA |
| | | | | insertion |
| | | | | site |
| At3g25140 | 8 | 102380 R | ggtttgttaatcagatccgtgtaattcct | iSECT |
| | | | | tools T- |
| | | | | DNA |
| | | | | insertion |
| | | | | site |
| At3g25140 | 8 | 041919 F | tctcttctaatgatctaatcccacaataa | iSECT |
| | | | | tools T- |
| | | | | DNA |
| | | | | insertion |
| | | | | site |
| At3g25140 | 8 | 041919 R | ggtttgttaatcagatccgtgtaattcct | iSECT |
| | | | | tools T- |
| | | | | DNA |
| | | | | insertion |
| | | | | site |
| At3g02350 | 9 | 135312 F | acagcctgttgtaacaaagcccata | iSECT |
| | | | | tools T- |
| | | | | DNA |
| | | | | insertion |
| | | | | site |
| At3g02350 | 9 | 135312 R | ctcgctgtcttcaccttatccttca | iSECT |
| | | | | tools T- |
| | | | | DNA |
| | | | | insertion |
| | | | | site |
| At3g02350 | 9 | 115588 F | tctctgataatgtcattgctgtgtctgtt | iSECT |
| | | | | tools T- |
| | | | | DNA |
| | | | | insertion |
| | | | | site |
| At3g02350 | 9 | 115588 R | tcatgtttccattgtaatgaatcactcct | iSECT |
| | | | | tools T- |
| | | | | DNA |
| | | | | insertion |
| | | | | site |
| At3g02350 | 9 | 040287 F | acacagcttaaaatccagaagttgaaaga | iSECT |
| | | | | tools T- |
| | | | | DNA |
| | | | | insertion |
| | | | | site |
| At3g02350 | 9 | 040287 R | agttaaacaatggacttaccaggttctgc | iSECT |
| | | | | tools T- |
| | | | | DNA |
| | | | | insertion |
| | | | | site |
| At2g20810 | 10 | 029319 F | ctcttctttctcattctctccaaagctg | iSECT |
| | | | | tools T- |
| | | | | DNA |
| | | | | insertion |
| | | | | site |
| At2g20810 | 10 | 029319 R | atgagaaatcctcgaacttctgaacct | iSECT |
| | | | | tools T- |
| | | | | DNA |
| | | | | insertion |
| | | | | site |
| At2g20810 | 10 | 082273 F | atgggtttttaaccaatacccgaattact | iSECT |
| | | | | tools T- |
| | | | | DNA |
| | | | | insertion |
| | | | | site |
| At2g20810 | 10 | 082273 R | agcaagagcaatctgatcattaacttgac | iSECT |
| | | | | tools T- |
| | | | | DNA |
| | | | | insertion |
| | | | | site |
| At1g18580 | 11 | 104761 F | ccaaatcaaacgaaatgaaagtagacaaa | iSECT |
| | | | | tools T- |
| | | | | DNA |
| | | | | insertion |
| | | | | site |
| At1g18580 | 11 | 104761 R | cgaacattagcagttataaacactcaccc | iSECT |
| | | | | tools T- |
| | | | | DNA |
| | | | | insertion |
| | | | | site |
| At1g18580 | 11 | 148781 F | tatttcgtttgatgaggctaaaccg | iSECT |
| | | | | tools T- |
| | | | | DNA |
| | | | | insertion |
| | | | | site |
| At1g18580 | 11 | 148781 R | tttcgatcagacggttatcgatgtt | iSECT |
| | | | | tools T- |
| | | | | DNA |
| | | | | insertion |
| | | | | site |
| At5g54690 | 12 | 044387 F | ggtttgcttcttgcttccgct | iSECT |
| | | | | tools T- |
| | | | | DNA |
| | | | | insertion |
| | | | | site |
| At5g54690 | 12 | 044387 R | tttgggacattgacatgaatgga | iSECT |
| | | | | tools T- |
| | | | | DNA |
| | | | | insertion |
| | | | | site |
| At5g54690 | 12 | 014026 F | ttttagtgagaatcgaatgttttgtc | iSECT |
| | | | | tools T- |
| | | | | DNA |
| | | | | insertion |
| | | | | site |
| At5g54690 | 12 | 014026 R | cttcaacataaagccaaatcctaaa | iSECT |
| | | | | tools T- |
| | | | | DNA |
| | | | | insertion |
| | | | | site |
| At3g01040 | 13 | 122602 F | aaaaggcttgatttttcttcttctcctct | iSECT |
| | | | | tools T- |
| | | | | DNA |
| | | | | insertion |
| | | | | site |
| At3g01040 | 13 | 122602 R | ccttaacttgatagttgaacaaaatgcca | iSECT |
| | | | | tools T- |
| | | | | DNA |
| | | | | insertion |
| | | | | site |
| At5g15470 | 14 | 000091 F | TTAAGTCTCCCTGGACAACTATATCAT | iSECT |
| | | | | tools T- |
| | | | | DNA |
| | | | | insertion |
| | | | | site |
| At5g15470 | 14 | 000091 R | CAATTGTCAAGTTGGTTTCTTTTCT | iSECT |
| | | | | tools T- |
| | | | | DNA |
| | | | | insertion |
| | | | | site |
| At5g15470 | 14 | 029525 F | ttgggtccgctactgatctga | iSECT |
| | | | | tools T- |
| | | | | DNA |
| | | | | insertion |
| | | | | site |
| At5g15470 | 14 | 029525 R | gcagtgatccactacaatgggc | iSECT |
| | | | | tools T- |
| | | | | DNA |
| | | | | insertion |
| | | | | site |
| At3g58790 | 15 | 113194 F | agcactatgtgcaagtgttgagattttt | iSECT |
| | | | | tools T- |
| | | | | DNA |
| | | | | insertion |
| | | | | site |
| At3g58790 | 15 | 113194 R | tgtttttgatgaactgatagtggagatca | iSECT |
| | | | | tools T- |
| | | | | DNA |
| | | | | insertion |
| | | | | site |
| At3g58790 | 15 | 117272 F | ttttctaaagaagccaagcggacat | iSECT |
| | | | | tools T- |
| | | | | DNA |
| | | | | insertion |
| | | | | site |
| At3g58790 | 15 | 117272 R | tgttatccacagctgacaatgtttttg | iSECT |
| | | | | tools T- |
| | | | | DNA |
| | | | | insertion |
| | | | | site |
| At3g58790 | 15 | 070957 F | tggcatctatagtaatccatacgacgatt | iSECT |
| | | | | tools T- |
| | | | | DNA |
| | | | | insertion |
| | | | | site |
| At3g58790 | 15 | 070957 R | ttgaatgctatgtgcttgtcatctttaat | iSECT |
| | | | | tools T- |
| | | | | DNA |
| | | | | insertion |
| | | | | site |
| | | Left Border | TGGTTCACGTAGTGGGCCATCG | pROK T- |
| | | a F | | DNA |
| | | | | insertion |
| | | | | seq |
| | | Left Border | GCGTGGACCGCTTGCTGCAACT | pROK T- |
| | | b F | | DNA |
| | | | | insertion |
| | | | | seq |
| | | Left Border | GGTGATGGTTCACGTAGTGGGCCATCGC | pROK T- |
| | | c F | | DNA |
| | | | | insertion |
| | | | | seq |

Isolation of Cell Walls

Cell wall samples were harvested from selected tissues of multiple 8-week-old plants from WT and mutant lines (n=4). The plant tissues for cell wall extraction were weighed (100-200 mg), flash frozen in liquid N2 and ground to a fine powder. The tissues were consecutively extracted with 2 ml of 80% (v/v) ethanol, 100% ethanol, chloroform:methanol (1:1, v/v), and 100% acetone. Centrifugation in a table-top centrifuge at 6000 g for 10 min was used to pellet the sample between all extractions. The remaining pellet was immediately treated with a-amylase (Sigma, porcine Type-I) in 100 mM ammonium formate pH 6.0. The resulting pellet was washed three times with sterile water, twice with acetone, and dried in a rotary speed-vac overnight at 40° C. and weighed.

Mucilage Extraction

Mucilage was extracted from 200 Arabidopsis seeds incubated with sterile water at 60° C. over the course of 6 h as follows. Each hour during the 6-h period, the seeds were centrifuged and the supernatant was transferred to a sterile tube. The combined supernatants were lyophilized and re-suspended in 600 microliter of sterile water. Phenol-sulfuric (Dubois et al., 1956, Anal. Chem. 28, 350-356) and m-hydroxybiphenyl (Blumenkrantz and Asboe-Hansen, 1973, Anal. Biochem. 54, 484-489) assays, to quantify total sugars and uronic acids, respectively, were carried out using 100 microliter of the mucilage extracts. Duplicate 200 microliter aliquots of the mucilage extract were used for glycosyl residue composition analyses. To analyze the seed coat material remaining after extraction, the water-extracted seeds were aliquoted in water to glass tubes and 20 microgram of inositol was added. The seeds were lyophilized to dryness and used for glycosyl residue composition analyses.

TMS GC-MS Glycosyl Residue Composition

The cell walls were aliquoted (1-3 mg) as acetone suspensions to individual tubes and allowed to air dry. Inositol (20 microgram) was added to each tube and the samples were lyophilized and analyzed for glycosyl residue composition by combined gas chromatography-mass spectrometry (GC-MS) of the per-O-trimethylsilyl (TMS) derivatives of the monosaccharide methyl glycosides produced from the sample by acid methanolysis basically as described by York et al. (1985, Methods Enzymol. 118, 3-40). The dry samples were hydrolyzed for 18 h at 80° C. in 1 M methanolic-HCl. The samples were cooled and evaporated under a stream of dry air and further dried two additional times with anhydrous methanol. The walls were derivatized with 200 mircrol of TriSil Reagent (Pierce-Endogen, Rockford, Ill., USA) and heated to 80° C. for 20 min. The cooled samples were evaporated under a stream of dry air, re-suspended in 3 ml of hexane, and filtered through packed glass wool. The dried samples were re-suspended in 150 microliter of hexane and 1 microliter of sample was injected onto an HP 5890 gas chromatograph interfaced to a 5970 MSD using a Supelco DB1 fused silica capillary column.

Statistical Analyses

The variance ratio test (α=0.05) was used to compare the variances of standards and samples. ANOVA analyses, standard deviation, variance, t, and the mean of sample were calculated using SAS 9.1.3 software (SAS Institute Inc., Cary, N.C., USA). Significant differences between WT and mutant compositions were determined with ta(2)=0.1 (90% confidence), but was set to 0.05 (95% confidence) for all other analyses. The appropriate sample size was predicted using equation 7.7, p. 105 of Biostatistical Analysis, 4th edn (Zar, 1999, Biostatistical Analysis, 4th edn (Englewood Cliffs, N.J.: Prentice Hall) (Table 2).

TABLE 2
Determination of the number of replicate TMS GC-MS samples
required or 90% or greater statistical confidence.

| sam- | GalA | mean | d = | n at | mean | d = | n at |
|---|---|---|---|---|---|---|---|
| ple^{a} | mol % | of 3 | 15%^{b} | t_{α(2)} = 0.1^{c} | of 4 | 15%^{b} | t_{α(2)} = 0.1^{c} |
| 1 | 22.69 | 20.98 | 3.15 | 4.82 | 20.25 | 3.04 | 2.80 |
| 2 | 21.98 | | | | | | |
| 3 | 18.28 | | | | | | |
| 4 | 18.05 | 16.32 | 2.45 | 3.24 | | | |
| 5 | 15.61 | | | | 18.47 | 2.77 | 0.90 |
| 6 | 15.29 | | | | | | |
| 7 | 22.35 | 22.10 | 3.31 | 1.45 | | | |
| 8 | 20.62 | | | | | | |
| 9 | 23.32 | | | | 19.56 | 2.93 | 3.17 |
| 10 | 15.40 | 18.30 | 2.74 | 7.65 | | | |
| 11 | 20.42 | | | | | | |
| 12 | 19.08 | | | | | | |
| 13 | 16.92 | 16.54 | 2.48 | 0.20 | 16.44 | 2.47 | 1.47 |
| 14 | 16.16 | | | | | | |
| 15 | 16.56 | | | | | | |
| 16 | 16.14 | 18.29 | 2.74 | 32.68 | | | |
| 17 | 24.40 | | | | | | |
| 18 | 14.33 | | | | | | |
| mean | 18.76 | | | | | | |

^{a}The arbitrarily assigned sample number for each independent replicate is listed with the corresponding GalA mole % composition used for the determination of the minimum number of replicates necessary for a statistical confidence of 90%. The data shown are from pooled walls of 10 week old inflorescence samples, although comparable variation was also obtained from leaf, silique, stem and inflorescence tissue samples from 8 week old plants.
^{b}‘d’ refers to a margin of difference from the mean of 15%. Analysis of WT walls showed that natural variation was within 15% of the mean. Variation greater than 15% was indicative of mutation-associated changes in wall composition. The equation used to calculate ‘d’ is: Sample size = n = 2(S²₁ * t²)/d² where n = sample size, d = [Xₐᵥₑ − (t − se)] = difference from mean, S² = (Xₐᵥₑ − Xᵢ)² = sum of squares and α = 0.05 for a 2 tailed analysis. X is the value of the sample in whatever units used and se = standard error.
^{c}‘n’ = the number of replicates necessary to obtain a 90% confidence level in a two tailed analysis (t_{α(2)} = t_{0.1(2)}). For example, if n > the actual number of replicates used in the analysis, then it is false that a 15% difference (d) can be detected with 90% confidence. In this analysis, when 3 replicates were used, n is greater than 3 in four out of six cases, which means that a 15% difference (d) was detected with 90% confidence in only 2 out of 6 experiments. Conversely, when 4 replicates were used, n was less than 4 in all experiments and thus a 15% difference was detected with 90% confidence in all experiments.

RNA Extraction and RT-PCR

Total RNA was extracted from 0.5 g of stem, inflorescence, silique, and leaf tissue from 8-week-old plants. The tissues were homogenized in 10 ml of Homogenization Buffer (2% (w/v) SDS in 50 mM Tris-HCl pH 7.8 and 40% water-saturated phenol) and shaken for 15 min at 25° C. Tissue samples were centrifuged for 10 min at 8000 g and 4° C., and the supernatant removed to a clean tube. The samples were extracted two times with phenol:chloroform:isoamyl alcohol (25:24:01, v/v) and the aqueous phases were pooled. RNA was precipitated overnight with 0.1 vol. of 3 M sodium acetate and 2.5 vol. of cold ethanol. The samples were DNase-treated with RQ1 RNase-Free DNase (Promega, Madison, Wis., USA) according to the manufacturer's instructions.

RT-PCR products were generated using primer sequences unique to each of the 15 GAUT genes (Table 2). Each GAUT gene primer set was designed to span at least one intron such that unique PCR products were produced from RNA for each GAUT gene. Control RT reactions were carried out alongside GAUT-specific reactions, utilizing primers designed to the small ribosomal protein L23 alpha, wherein the primers do not produce a product in genomic DNA (Volkov et al., 2003, J. Exp. Bot., 54, 2343-2349). Qualitative RT-PCR was carried out using 5 lg of total RNA in a 20-microliter RT first-strand synthesis reaction that contained oligo(dT) primers. The RT first-strand reaction (2 microliter) was added to a PCR reaction mix containing the respective GAUT gene-specific primers and amplified for 30 cycles. Semi-quantitative RT-PCR was done using 2 microgram of total RNA in a 20-microliter RT first-strand synthesis reaction containing oligo(dT) primers. An aliquot (1.5 microliter) of the RT first-strand reaction was amplified through 26 cycles of PCR using GAUT genespecific primers. The PCR parameters were: Step 1: 95° C. for 5 min; Step 2: 95° C. for 0.5 min; Step 3: 55° C. for 0.5 min; Step 4: 72° C. for 1.5 min; Step 5: Return to step 2 (29 or 25) times; Step 6: 72° C. for 2 min; and Step 7: 4° C. forever.

TABLE 3
The Arabidopsis GAUT Family and T-DNA Insertion Seed Lines.

| Locus | Gene | Clade^{a} | I/S^{b} | SALK | Mutant Name | L^{c} | KO/KD/W^{d} |
|---|---|---|---|---|---|---|---|
| At3g61130 | GAUT1 | A-1 | 100/100 | | | | Not available |
| At2g46480 | GAUT2 | A-1 | 65/78 | 122209 | gaut2-1 | P | Not detected |
| At4g38270 | GAUT3 | A-1 | 68/84 | 001920 | gaut3-1 | I | KO |
| | | | | 113167 | gaut3-2 | 5′ | KD |
| At5g47780 | GAUT4 | A-2 | 66/83 | 034472 | gaut4-1 | 5′ | Not recovered |
| | | | | 001026 | gaut4-2 | 5′ | Not recovered |
| At2g30575 | GAUT5 | A-3 | 45/67 | 050186 | gaut5-1 | E | KO |
| | | | | 058223 | gaut5-2 | P | KD |
| At1g06780 | GAUT6 | A-3 | 46/64 | 007987 | gaut6-1 | E | KO |
| | | | | 056646 | gaut6-2 | E | KO |
| | | | | 073484 | gaut6-3 | 5′ | KD |
| At2g38650 | GAUT7 | A-4 | 36/59 | 015189 | gaut7-1 | E | KD |
| | | | | 046348 | gaut7-2 | P | KD |
| At3g25140 | GAUT8 | B-1 | 58/77 | 030075 | gaut8-1 | 3′ | KD |
| | | | | 039214 | gaut8-2 | E | HM lethal |
| | | | | 041919 | gaut8-3 | I | HM lethal |
| | | | | 102380 | gaut8-4 | I | HM lethal |
| At3g02350 | GAUT9 | B-1 | 57/76 | 135312 | gaut9-1 | E | W |
| | | | | 115588 | gaut9-2 | E | W |
| | | | | 040287 | gaut9-3 | E | KD |
| At2g20810 | GAUT10 | B-2 | 50/72 | 029319 | gaut10-1 | E | KO |
| | | | | 082273 | gaut10-2 | E | KD |
| At1g18580 | GAUT11 | B-2 | 51/71 | 104761 | gaut11-1 | 5′ | KD |
| | | | | 148781 | gaut11-2 | 3′ | KD |
| At5g54690 | GAUT12 | C | 40/61 | 044387 | gaut12-1 | I | KO |
| | | | | 014026 | gaut12-2 | E | KO |
| | | | | 038620 | gaut12-5 | P | HM lethal |
| At3g01040 | GAUT13 | C | 43/62 | 122602 | gaut13-1 | E | W |
| At5g15470 | GAUT14 | C | 43/62 | 000091 | gaut14-1 | E | KO |
| | | | | 029525 | gaut14-2 | 3′ | KO |
| At3g58790 | GAUT15 | C | 37/56 | 113194 | gaut15-1 | I | W |
| | | | | 117272 | gaut15-2 | P | W |
| | | | | 070957 | gaut15-3 | I | KO |

^{a}GAUT clades based on phylogenetic analysis (Sterling et al., 2006, PNAS USA, 103, 5236-5241).
^{b}The amino acid sequence identity and similarity (I/S) of each GAUT gene to GAUT1 (Sterling et al., 2006, PNAS USA, 103, 5236-5241).
^{c}The tentative location of the T-DNA insertion site is in one of the following gene structures; exon (E), 5′ untranslated region (5′), intron (I), promoter (P), or 3′ untranslated region (3′).
^{d}Transcript levels of GAUT T-DNA insertion mutant lines: Knockout, KO; Knockdown, KD; WT-like, W. Transcript for GAUT2 was not detectable in WT; therefore, the status of the mutant transcript was not able to be determined.

Mutant transcript levels were assessed as follows: knockouts (KO) were defined as mutants with RT-PCR reactions that yielded no detectable PCR product using gene-specific primers. Knockdown (KD) mutants were those that yielded a PCR product with significantly decreased intensity compared to the WT.

### Results

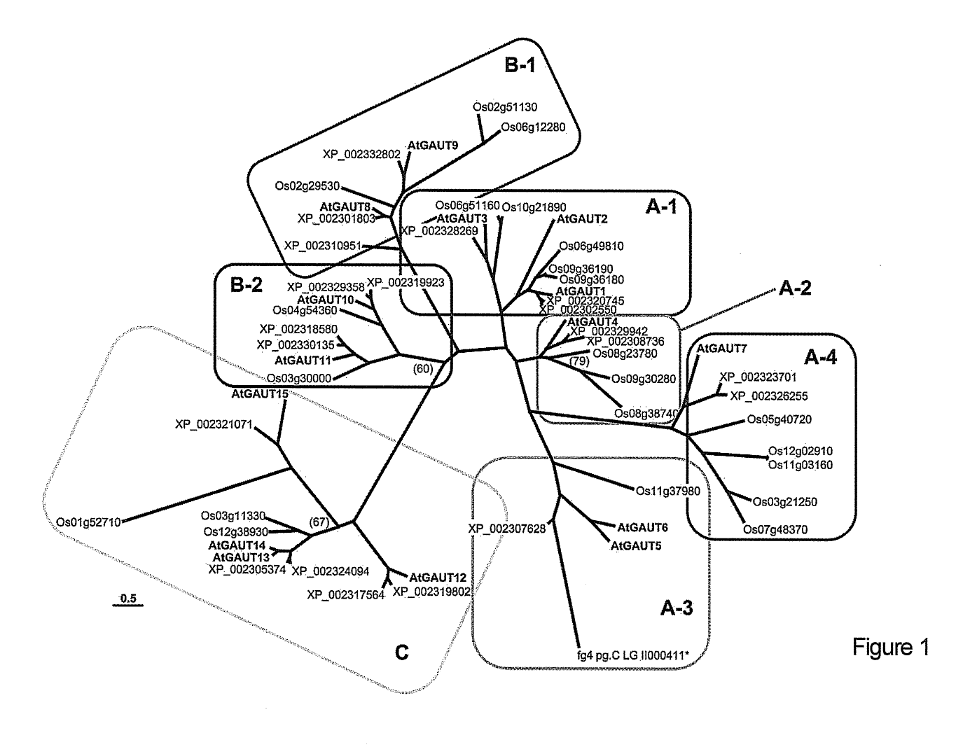

The GAUT Family of Arabidopsis, Poplar, and Rice The Arabidopsis GAUT1-related gene family encodes 15 GAUT and 10 GATL proteins with 56-84 and 42-53% amino acid sequence similarity, respectively, to GAUT1 (Sterling et al., 2006, PNAS USA, 103, 5236-5241). Previous phylogenetic analyses of the Arabidopsis GAUT1-related gene family resulted in the designation of three GAUT clades, clades A through C, and one GATL clade (Sterling et al., 2006, PNAS USA, 103, 5236-5241). The GATL clade, which consists of genes that cluster tightly and somewhat independently of the GAUT genes, was not included in the study reported here. It was previously determined that some Arabidopsis GAUT genes had conserved orthologs among species of both vascular and non-vascular plants (Sterling et al., 2006, PNAS USA, 103, 5236-5241). The genomes of rice (Oryza sativa) and poplar (Populus trichocarpa) have now been sequenced and a BLAST search of Arabidopsis GAUT motifs against the poplar and rice genomes revealed GAUT1-related gene families of 21 members in poplar and 22 members in rice (FIG. 1). Due to a recent genome duplication event in Populus (Tuskan et al. 2006, Science. 313, 1596-1604), there are one to two apparent poplar orthologs for each Arabidopsis GAUT. A similar distribution of GAUTs in poplar and Arabidopsis is observed, except for the absence of a GAUT2 ortholog in poplar. In contrast, rice has major distinctions from Arabidopsis and poplar in the distribution of GAUT gene orthologs. Rice does not have apparent orthologs of GAUT2 or GAUT12. In addition, there are multiple apparent isoforms of GAUTs 1, 4, 7, and 9, suggesting an expansion of the role of these GAUT genes in rice.

The rice and poplar genes included in this comparative phylogenetic analysis resolved the GAUT genes into seven clades. In order to preserve previous clade identity between the original three Arabidopsis clades (Sterling et al., 2006, PNAS USA, 103, 5236-5241) and the more finely resolved seven clades presented here, the following clade identities are assigned. Arabidopsis GAUT clade A is subdivided into clades A-1, A-2, A-3, and A-4; GAUT clade B is subdivided into clades B-1 and B-2; and GAUT clade C remains undivided. The corresponding GAUTs in each clade are: A-1 (1 to 3); A-2 (4), A-3 (5 and 6) and A-4 (7); B-1 (8 and 9), B-2 (10 and 11) and C (12 to 15).

GAUT Gene Transcript Expression in Arabidopsis Tissues

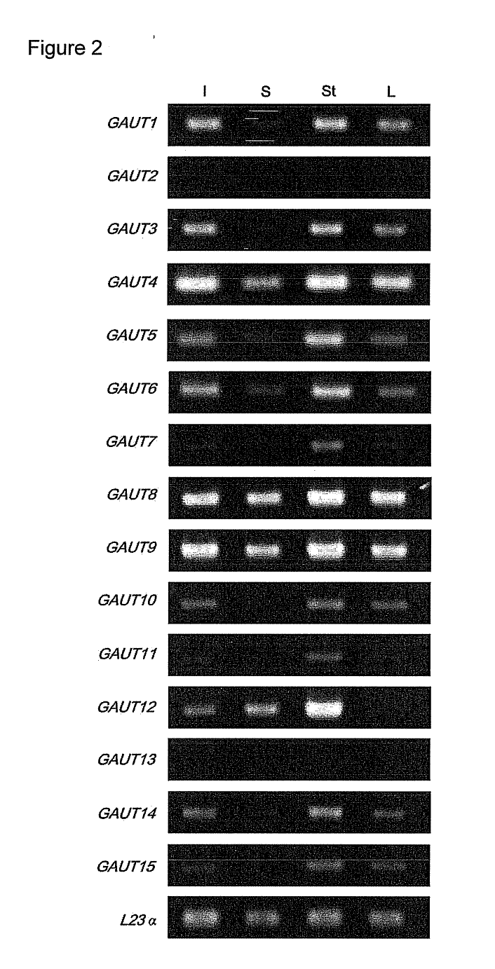

Available transcript expression of AtGAUTs compiled from the Whole Genome Array, Massively Parallel Signature Sequence, and Genevestigator bioinformatic databases (Table 4) was used to select tissues used for the cell wall analyses reported here. In addition, total RNA from 8-week-old Arabidopsis WT inflorescence, silique, stem, and leaf tissues was used for qualitative and semi-quantitative RT-PCR using GAUT genespecific primers. PCR products corresponding to the transcripts of 14 GAUT genes, excluding GAUT2, were detected in the WT inflorescence, leaf, stem, silique, and root tissues tested. GAUT2 may be expressed at a very low level or at different stages of development that have not yet been tested (FIG. 2). Qualitative RT-PCR results partially agree with the published transcript expression data (see Table 4). In several instances, we detected GAUT transcript in tissues where it had not been previously reported. The data available from the Whole Genome Analysis (Yamada et al., 2003, Science. 302, 842-847) did not detect GAUT5, while the Massively Parallel Signature Sequence data did not indicate detection of GAUTs 7, 10, 11, and 12 in leaf, GAUTs 1, 3, and 7 in stem, and GAUTs 1, 3, 4, 8, 9, 10, 13, and 15 in silique (Meyers et al., 2004, Plant Physiol., 135, 801-813). Overall, the data supplied by Whole Genome Analysis and Massively Parallel Signature Sequences under-reported GAUT gene transcript expression. The relative transcript expression of the GAUT genes, however, more closely agrees with that reported by Genevestigator (Zimmermann et al., 2004, Plant Physiol. 136, 2621-2632). Genevestigator does not list a probe for GAUT5, and therefore has no expression data for this gene, while the MPSS database reports low to moderate expression of GAUT5, in agreement with the result reported here.

TABLE 4
Bioinformatic Arabidopsis GAUT Gene Transcript Expression Data.

| Locus | | | | | | | | | | | |
|---|---|---|---|---|---|---|---|---|---|---|---|
| potential^{d} | Gene^{a} | WGA^{b} | INF^{c} | LEF | LES | ROF | SIF | SIS | CAF | CAS | Expression |
| At3g61130 | GAUT1 | + | 114 | 48 | 46 | 42 | 22 | 25 | 18 | 0 | 14 093 |
| At2g46480 | GAUT2 | − | 0 | 0 | 0 | 0 | 0 | 0 | 0 | 0 | 1493 |
| At4g38270 | GAUT3 | + | 0 | 11 | 12 | 2 | 13 | 58 | 31 | 50 | 6851 |
| At5g47780 | GAUT4 | + | 87 | 161 | 0 | 142 | 154 | 0 | 152 | 0 | 18 061 |
| At2g30575 | GAUT5 | − | 11 | 19 | 1 | 14 | 7 | 18 | 5 | 20 | — |
| At1g06780 | GAUT6 | + | 0 | 4 0 | 0 | 0 | 0 | 0 | 0 | 11 | 224 |
| At2g38650 | GAUT7 | + | 68 | 69 | 111 | 62 | 40 | 218 | 53 | 236 | 7126 |
| At3g25140 | GAUT8 | + | 405 | 125 | 72 | 230 | 285 | 664 | 117 | 329 | 27 875 |
| At3g02350 | GAUT9 | + | 74 | 78 | 28 | 450 | 249 | 106 | 93 | 69 | 15 384 |
| At2g20810 | GAUT10 | + | 39 | 29 | 50 | 42 | 13 | 0 | 42 | 0 | 7087 |
| At1g18580 | GAUT11 | + | 19 | 1 | 5 | 22 | 29 | 38 | 17 | 26 | 12 6915 |
| At5g54690 | GAUT12 | + | 44 | 5 | 2 | 19 | 37 | 3 | 0 | 0 | 12 028 |
| At3g01040 | GAUT13 | + | 24 | 11 | 8 | 58 | 4 | 1 | 22 | 10 | 9670 |
| At5g15470 | GAUT14 | + | 5 | 14 | 15 | 25 | 4 | 46 | 3 | 9 | 5386 |
| At3g58790 | GAUT15 | + | 0 | 0 | 0 | 0 | 16 | 0 | 4 | 12 | 6717 |

^{a}GAUT gene designation (Sterling et al., 2006, PNAS USA, 103, 5236-5241)
^{b}Expression of GAUT gene transcript was detected (+) or not (−) according to the Whole Genome Analysis (WGA) of Arabidopsis (Yamada et al., 2003, Science. 302, 842-847).
^{c}Relative expression of the designated GAUT gene transcript in different tissues, available through the Massively Parallel Signature Sequences (MPSS) website (http://mpss.udel.edu/at/) (Meyers et al., 2004, Plant Physiol., 135, 801-813): INF (Inflorescence-mixed stage, immature buds, classic MPSS), LEF (Leaves-21 d, untreated, classic MPSS), LES (Leaves-21 d, untreated), ROF (Root-21 d, untreated, classic MPSS), SIF Silique-24-48 h post-fertilization, classic MPSS), SIS (Silique-24-48 h post-fertilization, signature MPSS), CAF (Callus-actively growing, classic MPSS), CAS (Callus-actively growing, signature MPSS).
^{d}GENEVESTIGATOR Expression Potential is the average of the top 1% signal value of a probe for the designated GAUT gene across all tissue expression arrays (Zimmermann et al., 2004, Plant Physiol. 136, 2621-2632).

In general, RT-PCR indicated that relative transcript expression in Arabidopsis was highest for GAUTs 1, 4, 8, 9, and 12, moderate for GAUTs 3, 5, 6, 10, 14, and 15, and low for GAUTs 2, 7, 11, and 13. It should be noted that RT-PCR of GAUT7 repeatedly produced two bands, one of the expected size and a minor band of a smaller size. Whether the smaller band represents a splice variant has not been investigated. The RT-PCR data indicated that the GAUT genes were expressed at some level in all tissues tested; therefore, inflorescence, silique, leaf, and stems were used for the chemical and biochemical studies of the GAUT mutants.

Isolation of Homozygous Mutants of 13 of the 15 GAUT Genes

Twenty-six Arabidopsis homozygous T-DNA insertion seed lines in 13 distinct GAUT genes were isolated from mutagenized seed obtained from the SALK Institute (http://signal.salk.edu/cgi-bin/tdnaexpress) through the Arabidopsis Biological Resource Center (Alonso et al., 2003, Science. 301, 653-657). Mutant seed lines were preferentially selected with the T-DNA insertion site in an exon, 5′ UTR, or intron of the GAUT gene, if such lines were available. SALK insertion seed lines of GAUT1 were not available and neither homozygous nor heterozygous mutants were recovered from the SALK insertion seed lines for GAUT4. RT-PCR of total RNA isolated from homozygous gaut mutant lines identified 10 knockout mutants and 10 knockdown mutants (Table 3).

Growth Phenotypes of gaut Mutants

The gaut mutants plants were initially inspected visually for obvious growth phenotypes, such as dwarfing and/or organ malformation, compared to WT plants. Major abnormalities were not observed in plant growth or morphology for most gaut mutants isolated in this study, with the exception of gaut8 and gaut12. The presence of subtle growth phenotypes may require more sensitive methods than those applied here. Indeed multiple stem elongation phenotypes are observed with multiple gaut mutants. Functional redundancy among the GAUT proteins may contribute to the lack of severe phenotypes observed among gaut mutants. Estimates put forth by Østergaard and Yanofsky (2004, Plant J. 39, 682-696) predict that mutations in only approximately 10% of genes may result in detectable mutant phenotypes due to gene redundancy among large gene families in higher organisms. Thus far, two out of 13 GAUT genes (;15%) have yielded mutants with severe growth phenotypes, which is in line with the predicted outcome (Østergaard and Yanofsky, 2004, Plant J. 39, 682-696).

Previously analyzed qua1-1 insertion mutants (insertion in the 5#UTR) had severe dwarfmg, sterility, and bumpy epidermal surfaces as a result of reduced cell adhesion (Bouton et al., 2002, Plant Cell, 14, 2577-2590). Mutants allelic to qua1-1 (gaut8-2, gaut8-3, and gaut8-4) produced only heterozygous and WT progeny, suggesting an embryo-lethal phenotype. A single homozygous mutant was isolated, gaut8-1, with a predicted insertion in the 3#UTR that did not show the expected qua1-1 phenotype and was experimentally determined to have detectable GAUT8 transcript by RT-PCR, which may account for the WT like phenotype of these plants.

The irx8-1/gaut12-1 and irx8-5/gaut12-2 mutant plants were severely dwarfed and sterile, which necessitated recovery of homozygous plants from the progeny of heterozygous parental plants, as previously reported (Persson et al., 2007, Plant Cell. 19, 237-255). The phenotype of irx8-1/gaut12-1 and irx8-5/gaut12-2 was recognized in plants at least 4 weeks old. Such plants were small and with darkened leaves compared to WT. Surprisingly, the gaut12-5 promoter mutant (SALK_{—}038620) did not produce homozygous progeny. In addition, gaut12-5 heterozygous mutants were dwarfed compared to WT, and more severely dwarfed compared to the irx8-1/gaut12-1 or irx8-5/gaut12-2 heterozygotes. RT-PCR of RNA from homozygous irx8-1/gaut12-1 and irx8-5/gaut12-2 plants did not yield PCR products using 5#- and 3#-end coding region-specific primers, showing that the full-length GAUT12 transcript was not produced. Because of the lethal phenotype, only heterozygous gaut12-5 was obtained and therefore was not included in our analyses of gaut homozygous mutants.

Strategy to Identify Glycosyl Residue Composition Differences between gaut Mutant and WT Walls

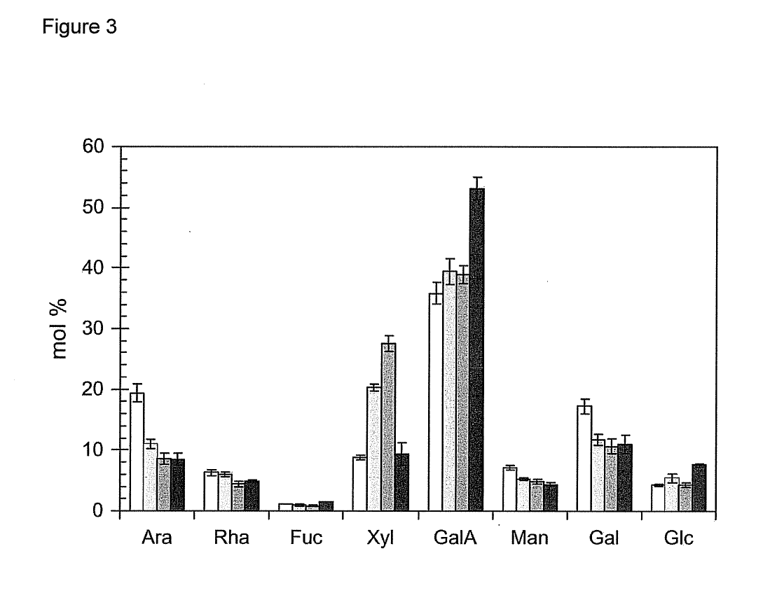

Gas chromatography-mass spectrometry (GC-MS) has been used to detect the changes in glycosyl residue composition in cell walls arising from mutations in cell wall-related genes (Reiter et al., 1997, Plant J. 12, 335-345). Analysis of wall glycosyl residue composition by GC-MS of trimethylsilyl (TMS) derivatives allows detection of acidic and neutral sugars in a single analysis (Doco et al., 2001), in contrast to composition analysis by formation of alditol acetate derivatives that detects neutral but not acidic sugars (Reiter et al., 1997, Plant J. 12, 335-345). Since uronic acids make up the largest proportion of glycosyl residues in the non-cellulosic wall polysaccharides of WT Arabidopsis tissues (FIG. 3), the TMS method was chosen to analyze gaut mutant walls. A statistical assessment of the TMS method showed that at least four independent TMS analyses per wall sample are necessary to detect a 15% difference between the glycosyl residue composition of different wall samples with 90% or greater statistical confidence (Table 2). The mutant glycosyl residue composition results were normalized to the composition of WT plants grown in the same experiment, in order to minimize the variability observed in the glycosyl residue compositions of plants grown in different experiments. Thus, for example, rhamnosyl compositions would be normalized according to the following foiinula:
Normalized Rha=[(mutant mol % Rha/WT mol % Rha)×100].

Normalization of mutant glycosyl residue composition to WT controls allowed mutant wall composition phenotypes to be compared between experiments. The tissues chosen for the cell wall analyses of each specific gaut mutant were based on transcript expression of the corresponding GAUTs in WT tissues according to the Whole Genome Array (Yamada et al., 2003, Science. 302, 842-847) and Massively Parallel Signature Sequences (Meyers et al., 2004, Plant Physiol., 135, 801-813) databases (see Table 4). To identify gaut mutant wall glycosyl residue compositions that were statistically different from those of WT walls, the normalized compositions were evaluated by ANOVA procedures (ta(2)=0.1). As an extra measure of stringency, a 15% point or greater departure from the normalized WT mean, in addition to a statistically different outcome by ANOVA, was required for declaration of a real difference from WT.

Wall Glycosyl Residue Composition is Altered in Multiple gaut Gene Mutants

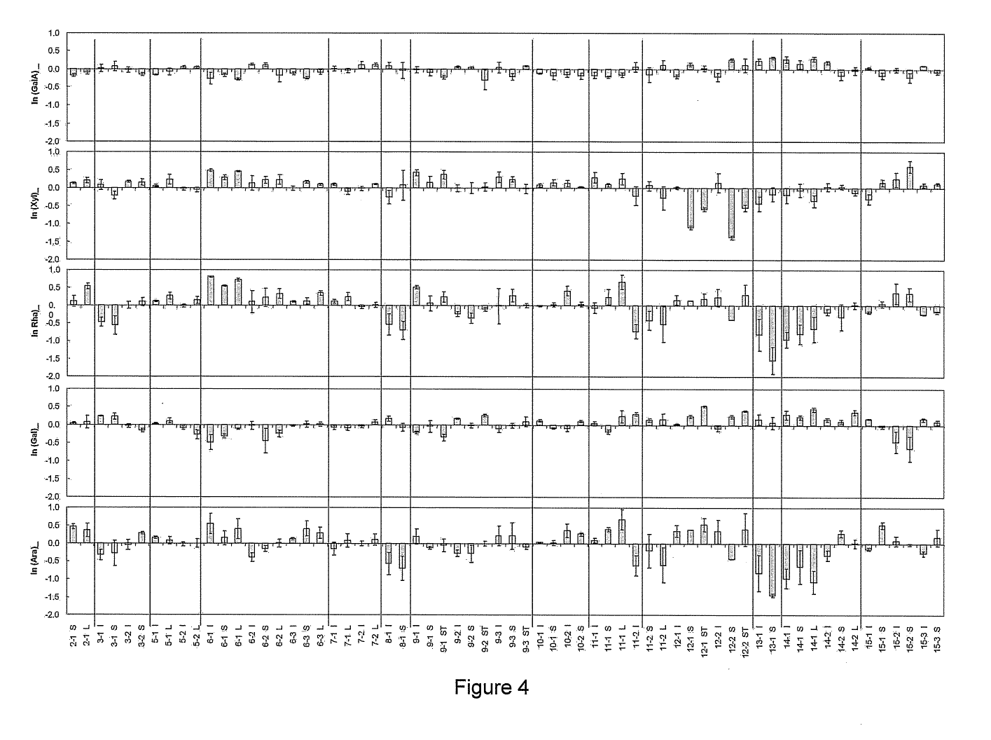

TMS glycosyl residue composition analyses of walls from two or more tissues of WTand mutant lines, representing 13 GAUT genes, revealed that specific gaut mutants have unique wall composition changes, which include increases and decreases in GalA, as well as significant changes in other glycosyl residues (Table 5). The wall glycosyl residue compositions that were statistically different in the gaut mutants compared to WT are shown in bold italics in Table 5. Reproducible mutant phenotypes were identified by comparing the natural log transformed data for all mutants that had statistically different mol % GalA, Xyl, Rha, Gal, and Ara levels compared to WT in at least two mutant alleles of the same gene or in at least two tissues of the same mutant allele (FIG. 4).

TABLE 5
Percent Cell Wall Glycosyl Residue Composition of
Arabidopsis gaut Mutants Compared to Wild-Type.^{a}
(mutant mol %/WT mol %*100)

| Mutant | Tissue^{b} | Ara | Rha | Fuc | Xyl | GalUA | Man | Gal | Glc |
|---|---|---|---|---|---|---|---|---|---|
| gaut2-1 | S | [symbol] | 116 | 108 | 114 | [symbol] | 103 | 103 | [symbol] |
| | L | 152 | [symbol] | 98 | [symbol] | 91 | 89 | 112 | 123 |
| gaut3-1 | I | 74 | [symbol] | [symbol] | 111 | 104 | 108 | [symbol] | [symbol] |
| | S | 90 | 62 | 72 | [symbol] | 110 | 104 | 126 | [symbol] |
| gaut3-2 | I | 99 | 102 | 181 | [symbol] | 99 | 89 | 97 | [symbol] |
| | S | 132 | 112 | 109 | 118 | 87 | 98 | 86 | [symbol] |
| gaut5-1 | I | 117 | 112 | 110 | 105 | 85 | [symbol] | 102 | 73 |
| | S | 109 | 132 | 117 | 130 | 94 | [symbol] | 112 | [symbol] |
| gaut5-2 | I | 98 | 99 | 97 | 97 | 106 | 103 | 93 | 97 |
| | S | 102 | 118 | 43 | 95 | 105 | [symbol] | 78 | [symbol] |
| gaut6-1 | I | 193 | [symbol] | 154 | [symbol] | 80 | 162 | [symbol] | 128 |
| | S | 123 | [symbol] | [symbol] | [symbol] | [symbol] | 141 | [symbol] | 153 |
| | L | 168 | [symbol] | [symbol] | [symbol] | 75 | 167 | 89 | 107 |
| gaut6-2 | I | [symbol] | 126 | 95 | 122 | 114 | 133 | 99 | [symbol] |
| | S | 87 | 137 | 108 | 126 | 112 | 150 | 73 | [symbol] |
| | L | 103 | [symbol] | 115 | 129 | 87 | 131 | 79 | 153 |
| gaut6-3 | I | 113 | 111 | 102 | 100 | 88 | 111 | 98 | 92 |
| | S | 161 | 114 | [symbol] | [symbol] | [symbol] | 104 | 103 | 86 |
| | L | 139 | [symbol] | 106 | 109 | 92 | 102 | 102 | 112 |
| gaut7-1 | I | 91 | 113 | 104 | 110 | 102 | 89 | 93 | 126 |
| | L | 114 | 130 | 117 | 90 | 96 | 107 | 93 | 114 |
| gaut7-2 | I | 100 | 96 | 87 | 98 | 114 | 89 | 96 | 105 |
| | L | 112 | 102 | 100 | 110 | 113 | 102 | 108 | 51 |
| gaut8-1 | I | [symbol] | [symbol] | [symbol] | 81 | 111 | 106 | 119 | [symbol] |
| | S | 59 | [symbol] | [symbol] | 137 | 102 | 102 | 95 | 111 |
| gaut9-1 | I | 130 | [symbol] | [symbol] | [symbol] | 99 | 159 | [symbol] | [symbol] |
| | S | 89 | 113 | 118 | 122 | 92 | [symbol] | 99 | 136 |
| | ST | 101 | 131 | [symbol] | [symbol] | [symbol] | 146 | [symbol] | 127 |
| gaut9-2 | I | [symbol] | [symbol] | [symbol] | 100 | 106 | 100 | [symbol] | 99 |
| | S | 82 | 72 | [symbol] | 103 | 104 | 282 | 99 | 85 |
| | ST | 100 | 90 | 96 | 105 | 81 | 58 | [symbol] | 106 |
| gaut9-3 | I | 139 | 130 | 151 | [symbol] | 108 | 102 | 91 | 114 |
| | S | 147 | 137 | [symbol] | [symbol] | [symbol] | 100 | 98 | 112 |
| | ST | 100 | 100 | 100 | 100 | 100 | 100 | 100 | 100 |
| gaut10-1 | I | 103 | 98 | [symbol] | 107 | 89 | 120 | 112 | 86 |
| | S | 103 | 103 | 110 | 116 | [symbol] | 113 | 92 | 108 |
| gaut10-2 | I | 152 | [symbol] | 128 | 115 | 87 | 94 | 92 | [symbol] |
| | S | [symbol] | 104 | 85 | 103 | [symbol] | 85 | 110 | [symbol] |
| gaut11-1 | I | 110 | 96 | 99 | 137 | 85 | 100 | 105 | 146 |
| | S | [symbol] | 135 | 125 | 109 | [symbol] | 86 | [symbol] | 117 |
| | L | 222 | [symbol] | 128 | 133 | 86 | 90 | 131 | 124 |
| gaut11-2 | I | [symbol] | [symbol] | [symbol] | 86 | 108 | 99 | [symbol] | 125 |
| | S | 110 | 73 | 76 | 108 | 91 | 112 | 114 | 95 |
| | L | 75 | 83 | [symbol] | 88 | 115 | 95 | [symbol] | 83 |
| gaut12-1 | I | 148 | 120 | 97 | 101 | [symbol] | 89 | 102 | 142 |
| | S | 147 | 115 | 112 | [symbol] | 114 | 100 | [symbol] | 121 |
| | ST | 179 | 124 | 130 | [symbol] | 103 | 66 | [symbol] | 132 |
| gaut12-2 | I | 163 | 137 | 105 | 130 | 82 | 80 | 91 | 115 |
| | S | 65 | 67 | [symbol] | [symbol] | [symbol] | 102 | [symbol] | 169 |
| | ST | 198 | 154 | 126 | [symbol] | 117 | 60 | 148 | 109 |
| gaut13-1 | I | 62 | [symbol] | [symbol] | [symbol] | [symbol] | 111 | 120 | 123 |
| | S | [symbol] | [symbol] | 117 | 89 | [symbol] | 99 | 110 | [symbol] |
| gaut14-1 | I | [symbol] | [symbol] | [symbol] | 88 | [symbol] | [symbol] | 135 | 113 |
| | S | 70 | [symbol] | [symbol] | 98 | 117 | 110 | [symbol] | 97 |
| | L | [symbol] | [symbol] | [symbol] | [symbol] | [symbol] | 81 | [symbol] | 78 |
| gaut14-2 | I | [symbol] | 84 | [symbol] | 105 | [symbol] | 90 | [symbol] | [symbol] |
| | S | 136 | 86 | 204 | 104 | 86 | 121 | 111 | 64 |
| | L | 102 | 102 | 61 | 88 | 98 | [symbol] | [symbol] | 98 |
| gaut15-1 | I | 87 | [symbol] | 89 | [symbol] | 104 | 105 | [symbol] | [symbol] |
| | S | [symbol] | 107 | 117 | 118 | 85 | 86 | 96 | [symbol] |
| gaut15-2 | I | 111 | [symbol] | 67 | 134 | 99 | 213 | 72 | [symbol] |
| | S | 98 | 147 | 90 | [symbol] | [symbol] | 156 | 60 | 112 |
| gaut15-3 | I | [symbol] | [symbol] | [symbol] | 109 | 111 | 109 | [symbol] | 98 |
| | S | 130 | [symbol] | 95 | 112 | 93 | 103 | 109 | 84 |

^{a}Data represent four independent TMS GC-MS reactions from four independent wall extractions. Residues are abbreviated according to FIG. 3. SALK T-DNA seed lines were unavailable for gaut1 and were unable to be isolated from SALK seed received for gaut4.
^{b}The walls used for glycosyl residue analysis were harvested from inflorescence (I), silique (S), leaf (L), and stem (ST).
^{c}Bold highlighted italicized values indicate mutant glycosyl residue compositions that were statistically and ±15% different from the WT mean.

Eight gaut mutants had statistically different mol% levels of GalA, Xyl, Rha, Gal, or Ara in at least two mutant alleles of the same gene or in at least two tissues of the same mutant allele compared to WT, resulting in distinguishable patterns of glycosyl residue composition changes in the walls of gaut mutants (summarized in Table 6). The silique tissues of gaut6-1 and gaut6-3 were consistently reduced in GalA, increased in Xyl, Rha, and Fuc, and similar to WT in Gal and Ara wall composition. Viable gaut8 homozygous knockout mutants were not isolatable, and, therefore, the wall composition of qua1-1 is used to establish a phenotype grouping for gaut8 mutants. The leaves of qua1-1 that were previously analyzed (Bouton et al., 2002, Plant Cell, 14, 2577-2590) were decreased in GalA and Xyl, but were not changed in Rha or other sugars. The gaut9-1 stems were reduced in wall GalA and increased in Xyl and Fuc. The gaut10-1, gaut10-2, and gaut11-1 were consistently reduced in silique GalA only. The irx8-1/gaut12-1 and irx8-5/gaut12-2 mutant stems were severely reduced in Xyl, coincident with elevated Ara, Rha, and Gal content. The gaut12-1 and gaut12-2 are analogous to irx8-1 and irx8-5, and, consequently, show similar stem glycosyl residue composition as previously reported (Brown et al., 2005, Plant Cell. 17, 2281-2295; Pena et al., 2007, Plant Cell., 19, 549-563; Persson et al., 2007, Plant Cell. 19, 237-255). Gaut13-1, gaut14-1, and gaut14-2 had increased GalA and Gal and reduced Xyl, Rha, Ara, and Fuc, with greater mol% changes in gaut14-1 (T-DNA insertion in an exon) than gaut14-2 (T-DNA insertion in the 3′ region). There were also some changes in Fuc, Man, and Glc in walls of several gaut mutants. For example, increased Fuc was observed in gaut6-1, gaut6-2, gaut6-3, gaut9-1, gaut9-2, and gaut9-3; decreased Fuc in gaut8-1, gaut11-2, gaut14-1, and gaut14-2; increased Man in gaut5-1 and gaut5-2; increased Glc in gaut3-1, gaut3-2, and gaut6-2; and decreased Glc in mutants of gaut5-1, gaut5-2, and gaut10-2. Few significant changes were found in the walls of gauts 2, 3, 5, 7, and 15, and those that did occur were not consistent between two or more mutants or in more than one tissue of a single mutant.

TABLE 6
Phenotypic Grouping of gaut Mutants.^{a}

| gaut | GalA | Xyl | Rha | Gal | Ara |
|---|---|---|---|---|---|
| 6 | Down | Up | Up | Down | No change |
| 8^{b} | Down | Down | No change | No change | No change |
| 9 | Down | Up | Variable | Variable | No change |
| 10 | Down | No change | No change | No change | No change |
| 11 | Down | No change | Variable | Variable | Variable |
| 12 | Up^{c} | Down | No change | Up | No change |
| 13 | Up | Down | Down | Up | Down |
| 14 | Up | Down | Down | Up | Down |

^{a}Changes in the relative amount of the designated glycosyl residues compared to WT.
^{b}Due to the lethality of gaut8 homozygous mutants, the qual-1 leaf compositions were used for the phenotypic grouping of gaut8 (Bouton et al., 2002, Plant Cell, 14, 2577-2590).
^{c}The GalA composition of gaut12 stems and siliques was increased, but was reduced in inflorescences.

Survey of Seed Mucilage Reveals GAUT11 Involved in Mucilage Extrusion

The seeds of myxospermous species, such as Arabidopsis, extrude mucilage from the seed coat epidermal cells when hydrated to protect against desiccation and to aid in seed dispersal. The mucilage of WT and gaut mutant seeds was investigated by ruthenium red staining as a facile method to determine whether specific GAUT genes are involved in mucilage polysaccharide extrusion or synthesis. The mucilage extruded from Arabidopsis seeds is enriched in the pectic polysaccharide RG-I, which efficiently binds ruthenium red stain due to the negative charge on the GalA residues in mucilage. This method has been successfully employed to identify mucilage or testa polysaccharide biosynthesis mutants (Western et al., 2001). The seed mucilage was evaluated by observing the staining intensity of mucilage and measuring the mucilage thickness under a dissecting microscope after application of aqueous 0.05% ruthenium red to the seeds of WT and the 26 gaut mutant lines. A single mutant (gaut11-2) was identified that displayed a reproducible reduced mucilage thickness phenotype compared to WT seed mucilage thickness.

Ruthenium red staining of WT and gaut11-2 seeds (FIG. 5A-5C) revealed that; 68% of gaut11-2 seeds had little extruded mucilage, while the remaining gaut11-2 seeds (˜32%) had reduced thickness of the mucilage layer to approximately half that of WT. Samples of WT and gaut11-2 seed were tested three separate times independently, with similar results obtained in seed derived from different parental plants (Table 7). Analysis of the uronic acid content of the hot water-extracted mucilage (WEM) of gaut11-2 and WT seed indicated that WEM of WT had 59 microgram uronic acid per 200 extracted seeds, while gaut11-2 mucilage had 48 microgram uronic acid per 200 extracted seeds (Table 6). The total carbohydrate extracted, as detected by a phenol sulfuric acid assay, was similar for WT and gaut11-2 WEM. This suggests that even though very little mucilage was observed by ruthenium red staining, a similar amount of carbohydrate was able to be extracted over several hours, but that the uronic acid content of that mucilage was reduced by 19%. The gaut11-2 WEM was subjected to glycosyl residue composition analysis (FIG. 5) and found to have statistically significant reductions in GalA and Xyl content and increases in Man and Gal content, as determined by ANOVA (t_{α2}=0.05). The glycosyl residue composition of residual gaut11-2 seed material that represents the remaining mucilage, some testa wall, and possibly some storage polysaccharide was also reduced in GalA (69%) and Gal (68%) and increased in Ara (110%), Man (128%), and Glc (138%) compared to WT.

TABLE 7
WT and gaut11.2 Mucilage Expansion and Uronic Acid Content.

| | Mucilage (% seeds)^{a} | | UA (ug UA/200 seeds)^{b} | |
|---|---|---|---|---|
| Experiment | WT | gaut11-2 | WT | gaut11-2 |
| Experiment # 1 | 92 | 16 | 59 | 46 |
| Experiment # 2 | 100 | 41 | 58 | 46 |
| Experiment # 3 | 87 | 39 | 56 | 45 |
| Experiment # 4 | | | 61 | 48 |
| Experiment # 5 | | | 58 | 53 |
| Average | 93.0 ± 7 | 31.8 ± 14 | 58.8 ± 2 | 47.8 ± 3 |
| | P = 2.3⁻³ | | P = 2.2⁻⁴ | |

^{a}The data are the average (%) seeds with expanded mucilage after staining with aqueous ruthenium red.
^{b}The data are the uronic acid content of hot water-extracted mucilage per 200 seeds of WT and gaut11-2 as assayed by the m-hydroxylbiphenyl reagent assay.

Newly Resolved GAUT Gene Clades in Arabidopsis, Poplar, and Rice

The relatedness of GAUT genes has been re-evaluated based on the analysis of phylogenetic relationships of Arabidopsis, poplar, and rice GAUT genes. This comparative phylogenetic analysis distinguished seven GAUT clades (FIG. 1), instead of three, as previously proposed by Sterling et al. (2006, PNAS USA, 103, 5236-5241). The previous Arabidopsis GAUT clade A that included AtGAUT1-GAUT7 has been subdivided into four clades; GAUT clade A-1 (AtGAUT1 through 3), GAUT clade A-2 (AtGAUT4), clade A-3 (AtGAUT5 and AtGAUT6), and GAUT clade (AtGAUT7). The former Arabidopsis clade B has been subdivided into GAUTclade B-1 (AtGAUT8 and AtGAUT9) and GAUT clade B-2 (AtGAUT10 and AtGAUT11). The former Arabidopsis GAUTclade C has not been subdivided and contains AtGAUT12 through AtGAUT15.

GAUT2 does not appear to have a direct ortholog in either rice or poplar. It is possible that GAUT2 may not be a complete copy of a GAUT1 duplication event, based on a shorter N-terminus compared to GAUTs 1-7; however, its length is comparable to the other GAUTs. GAUT2 also does not have detectable transcript in the tissues tested and GAUT2 T-DNA insertion mutants did not have reproducible phenotypes. These data, combined with the phylogenetic analysis of GAUT2, support the hypothesis that GAUT2 may be a nonfunctional truncated homolog. It cannot be ruled out, however, that GAUT2 may have a very low abundance transcript and a unique function in Arabidopsis alone, although this seems unlikely based on the current data.

The Arabidopsis and poplar genomes have one (At2g38650) and two (XP_{—}002323701, XP_{—}002326255) copies of GAUT7, respectively, while the rice genome contains five GAUT7-like sequences. There is considerable evidence that the AtGAUT7 protein resides in a complex with AtGAUT1, a complex that has homogalacturonan a1,4-GalAT activity. GalAT activity was detected in immunoprecipitates from HEK cells transiently transfected with GAUT1, but not in HEK cells transiently transfected with GAUT7 (Sterling et al., 2006, PNAS USA, 103, 5236-5241). Based on these data, GAUT7 may be expressed in an inactive state with limited activity itself or may function as an ancillary protein necessary for GAUT1-associated GalAT activity. Whatever the role of GAUT7, its function appears to be dramatically expanded in rice. Because the role of GAUT7 in wall polysaccharide biosynthesis is currently unknown, the underlying biological reason for five copies of GAUT7 in rice remains to be determined.

Poplar and rice each have putative orthologs of GAUT9: XP_{—}002332802 (poplar), Os06g12280 (rice), and Os02g51130 (rice). Poplar also has at least one putative ortholog of GAUT8 (XP_{—}002301803). There is not an obvious ortholog of GAUT8 in rice, although there is one rice gene (Os02g29530) positioned between GAUT8 and GAUT9. Phylogenic analyses using additional sequenced plant genomes may clarify the relatedness of the latter gene to GAUT8 and GAUT9.

GAUT12 has two poplar orthologs but no orthologs in rice (FIG. 1). GAUT12 has been linked xylan synthesis. The putative functions that have been hypothesized for GAUT12 include an a1,4-GalAT that adds GalA into a primer or cap for xylan synthesis or as a novel linkage in xylan or pectic polysaccharides (Brown et al., 2005, Plant Cell. 17, 2281-2295; Pena et al., 2007, Plant Cell., 19, 549-563; Persson et al., 2007, Plant Cell. 19, 237-255). GAUT12 has been shown to be essential for normal growth and more specifically for the synthesis of secondary wall glucuronoxylan and/or wall HG synthesis. Rice does not have an apparent homolog of GAUT12, and appears to produce secondary wall xylan and glucuronoarabinoxylan, but not 4-O-methylglucuronoxylan (Ebringerova and Heinze, 1999, Macromol. Rapid Commun. 21, 542-556). Thus, GAUT12 may have a specialized function in glucuronoxylan synthesis of dicot plants. GAUT12transcript has been shown to be localized closely with glucuronoxylan-rich vascular tissues, suggesting that GAUT12 has a specialized role in the synthesis of secondary wall glucuronoxylan of dicot walls (Persson et al., 2007, Plant Cell. 19, 237-255). GAUT12 has an expression profile distinct from that of other GAUT genes according to semi-quantitative RT-PCR; it is much more highly expressed in stem than in other tissues compared to other GAUT transcripts. The unique transcript expressionprofile, role in secondarywall 4-O-methylglucuronoxylan synthesis, and exclusivity among the dicot species suggest that GAUT12 has undergone a differentiation that has rendered it essential in dicots and nonessential in monocots.

GAUT Gene Transcripts are Expressed Ubiquitously in Arabidopsis Tissues

The transcript expression of GAUT8 and GAUT12 has been associated with vascular tissues in Arabidopsis stem (Orfila et al., 2005, Planta. 222, 613-622; Persson et al., 2007, Plant Cell. 19, 237-255). The GAUT12 results described here agree with previous analyses of GAUT12/IRX8 gene expression by RT-PCR analysis (Persson et al., 2007, Plant Cell. 19, 237-255) and GAUT8 RT-PCR data agree with reports of QUA1 expression (by Northern blot) in ‘Flowers II’ and ‘Rosette Leaves II’ RNA, but do not agree with the low transcript expression reported in ‘Stems II’ by Bouton and colleagues (2002, Plant Cell, 14, 2577-2590). We report high relative expression of GAUT8 in stems. In situ PCR of QUA1/GAUT8 in WT stems (Orfila et al., 2005, Planta. 222, 613-622), however, did reveal prominent expression in that tissue, which is more closely aligned with our results. The detectable expression of all of the GAUT genes in all of the tissues tested correlates with a function in wall biosynthesis, as this is a process required by all plant cells. GUS reporter gene studies have shown that QUA2, a putative pectinmethyltransferase involved in pectin biosynthesis, also has ubiquitous expression (Mouille et al., 2007, Plant J. 50, 605-614).

The Wall Compositions of Multiple gaut Mutants are Altered Compared to WT

Analysis of the walls of gaut mutants using the TMS method (Doco et al., 2001, Carbohydr. Polym., 46, 249-259) allowed the GalA content of the walls to be quantified. An accurate quantification of wall GalA content is important when attempting to identify mutants of putative pectin biosynthesis genes, because GalA is a major component of the pectic polysaccharides (Ridley et al., 2001, Phytochemistry, 57, 929-967). Mutants of GAUTs 6, 9, 10, and 11 had statistically significant reductions in GalA content in more than one mutant sampling. Two other gaut mutants, gaut13 and gaut14, had statistically significant increased wall GalA content. The wall compositional phenotypes of the gaut mutants are discussed below.

The wall glycosyl residue composition phenotype of gaut6 provides compelling evidence that GAUT6 is a putative pectin biosynthetic GalAT. GAUT6 has 64% amino acid similarity to GAUT1 and gaut6 has reduced wall GalA that coincides with higher levels of Xyl and Rha wall compositions. It is possible that the increased Xyl and Rha content signifies the compensatory reinforcement of the wall by xylans and an apparent enrichment of RG-I in proportion to reduced HG polymers. Further work is necessary to test this hypothesis; however, preliminary results are in agreement with this hypothesis (Caffall, K. H., Ph.D. thesis, University of Georgia, 2008).

GAUTs 8, 9, 10 and 11 have been placed in two separate subclades (B-1 and B-2). However, all mutants in the two B clades show marked reductions in wall GalA content. Qual-1 mutant plants have walls with both reduced GalA and Xyl, and microsomal membrane protein preparations from qual-1 stems had reduced GalAT and xylan synthase activity compared to WT (Orfila et al., 2005, Planta. 222, 613-622; Brown et al., 2007, Plant J., 52, 1154-1168). The QUAl cumulative experimental evidence argues in favor of a putative pectin biosynthetic GalAT, based on the significant reduction in homogalacturonan and the strong defect in cell adhesion (Bouton et al., 2002, Plant Cell, 14, 2577-2590; Leboeuf et al., 2005, J. Exp. Bot., 56, 3171-3182). Deficiencies in cell adhesion have been associated with changes in pectin synthesis (Iwai et al., 2002, PNAS USA. 99, 16319-16324) and pectin localization (Shevell et al., 2000, Plant Cell. 12, 2047-2059). In addition, the transcript expression of a pair of Golgi-localized putative pectinmethyltranserfases is strongly correlated with QUA1/GAUT8 expression, as well as with the expression of GAUT9 and GAUT1 (Mouille et al., 2007, Plant J. 50, 605-614). The gaut9, gaut10, and gaut11 mutant plants did not have any obvious physical growth or cell adhesion defects, but the wall compositional phenotypes of these gaut plants, and the high amino acid similarity with QUA1/GAUT8, suggest that these GAUTs are putative pectin biosynthetic GalATs. The mutant alleles of GAUT9, GAUT10, and GAUT11 have reduced wall GalA content but were not decreased in Xyl, which has been observed in some mutants thought to be involved in xylan synthesis (Brown et al., 2007, Plant J., 52, 1154-1168; Lee et al., 2007; Pena et al., 2007, Plant Cell., 19, 549-563; Persson et al., 2007, Plant Cell. 19, 237-255). Based on the evidence, a role for the genes in GAUT clades A as well as a role for the genes in clade B and C in pectin biosynthesis is proposed.

In contrast to QUA1/GAUT8, IRX8/GAUT12 is believed to function in glucuronoxylan synthesis essential for secondary wall function. The irx8-1/gaut12-1 and irx8-5/gaut12-2 mutant plants have reduced Xyl content with increases in the GalA content in stem and silique walls, consistent with previous reports and consistent with the proposed function of IRX8/ GAUT12 in the synthesis of an oligosaccharide essential for xylan synthesis. Mutants of IRX8/GAUT12 and other putative xylan biosynthetic genes, IRX7, IRX8, IRX9, IRX14, and PARVUS, have similar wall compositional phenotypes (Pena et al., 2007, Plant Cell., 19, 549-563; Persson et al., 2007, Plant Cell. 19, 237-255). IRX8/GAUT12 may play a specialized role, among the GAUTs, in secondary wall synthesis and vascularization in dicot species (Brown et al., 2007, Plant J., 52, 1154-1168). Xylans are abundant in stem and silique tissues, where the Xyl compositional phenotype is observed; however, reductions in Xyl are not observed in inflorescence where IRX8/GAUT12 is also expressed. In inflorescences, irx8/gautl2 mutants show a reduction in GalA to 82% that of WT. Thus, the changes brought about by the lesion in GAUT12 additionally impact the pectin component of the wall. The underlying causes for the reduced GalA content in the inflorescence may be of significance to understand how pectin and xylan synthesis are regulated and connected.

The walls of gaut13 and gaut14 have increased GalA and Gal content and reduced Xyl and Rha content compared to WT. It seems unlikely that a mutant showing an increased wall GalA phenotype is involved in the synthesis of HG. However, reduced Rha, primarily a component of RG-I, may lead to walls enriched in HG, driving up GalA content. A Gal containing wall component is increased in the walls of gaut13 and gaut14 (and also gaut12). Pectic galactans have been associated with wall strengthening (McCartney et al., 2000) and are also increased in irx8/gaut12 walls (Persson et al., 2007, Plant Cell. 19, 237-255). A galactan in gaut13 and gaut14 may be up-regulated in response to wall weakening in a similar manner. GAUT13 and GAUT14 are very closely related to GAUT12, which would also suggest that the Xyl containing polysaccharide that is reduced in mutants of these genes is also a xylan and that GAUT13 and GAUT14 share overlapping function with GAUT12. Based on the strong transcript expression of GAUT12, most notably in the stem tissues of 8-week-old Arabidopsis plants, it is conceivable that gaut13 or gaut14, which have WT-like growth phenotypes, may be partially rescued by existing GAUT12 expression, if function is shared between GAUT12, GAUT13, and GAUT14, thus resulting in mild or undetectable growth phenotypes.

GAUT11 Effects Mucilage Extrusion

The composition and linkage analysis of gaut11-2 mucilage suggests a minor reduction in RG-I-like extractable polysaccharides. The gaut11-2 mutant has reduced mucilage expansion and reduced GalA content of extracted mucilage and testa, suggesting a role in the synthesis of mucilage polysaccharides. The gaut11-2 mutant has reduced GalA in silique walls, while gaut11-1 has reduced GalA in inflorescence, silique, and leaf walls. The gaut11-1 seeds, however, did not appear to have inhibited mucilage expansion. The predicted insertion site location of the T-DNA insertion present in gaut11-2 is in the 3#UTR, a location that may alter the targeting or regulation of GAUT11 expression rather than knocking out function (Lai, 2002) and account for the difference in phenotype between gaut11-1 and gaut11-2. The visible phenotype of gaut11-1 is similar in character to the mucilage modified (mum) mutants (Western et al., 2001, 2004). Three types of mum mutants have been described: mutants of pectin modification (mum2 and mum1), mutants affecting cytoplasmic rearrangement (transparent testa glabra-1; ttg1, glabra-2; g12), and mutants of mucilage biosynthesis (mum3, mum5, and mum4) (Western et al., 2001). Preliminary data suggest a role for GAUT11 in wall modification or biosynthesis based on the reduction in GalA in the extractable mucilage and based on the observation that the majority of the polysaccharides may be extracted over time, but are inefficiently released from the seed epidermal cells. It is known that unbranched RG-I, or reductions in intact RG-I, may lead to increased Ca2+cross-linking of HG in the wall (Jones et al., 2003, PNAS USA, 100, 11783-11788), and thus inhibit expansion and release of mucilage by hydration. Additionally, accumulation of less RG-I in the epidermal cells of the seed coat may prevent extrusion of the mucilage by reducing the internal pressure that is required to break through the epidermal cell wall necessary to release mucilage (Western et al., 2000, Plant Physiol., 122, 345-355).

Lethality of gaut Mutants: Something Lost, Something Gained

GAUT1 is an HG-GalAT. GAUT1 was the most abundant glycosyltransferase isolated from Arabidopsis suspension culture microsomal membrane fractions (Sterling et al., 2006, PNAS USA, 103, 5236-5241). In addition, GAUT1 and GAUT4 are expressed highly in the tissues of 8-week-old plants according to semi-quantitative RT-PCR and to the GENEVESTIGATOR and MPSS databases (FIG. 2 and Table 1) (Meyers et al., 2004, Plant Physiol., 135, 801-813; Zimmermann et al., 2004, Plant Physiol. 136, 2621-2632). Proteins that share high amino acid similarity often have a similar function and it is likely that GAUT4 (83% amino acid similarity to GAUT1) also has a function in synthesizing HG in the walls of Arabidopsis similar to that of GAUT1. The lack of recoverable mutants for GAUT1 and GAUT4 may speak to the importance of these genes in plant growth and development. Indeed, a gautl SAIL mutant yielded only heterozygous and WT progeny; homozygotes were not obtained. More vigorous attempts to isolate and characterize GAUT1 and GAUT4 and their respective mutants will undoubtedly aid in the clarification of their roles in pectin and wall biosynthesis. A degree of lethality has also been demonstrated in gaut8 and gaut12 mutants, both in this report and elsewhere (Bouton et al., 2002, Plant Cell, 14, 2577-2590; Persson et al., 2007, Plant Cell. 19, 237-255). Qual-1, irx8-1, and irx8-5 mutants are severely dwarfed and semi-sterile (Brown et al., 2005, Plant Cell. 17, 2281-2295; Orfila et al., 2005, Planta. 222, 613-622).

The data presented establish the foundation for multiple hypotheses regarding GAUT gene function. The rigorous testing of these hypotheses is expected to lead to the identification of additional genes involved in specific pectin and wall biosynthetic pathways. The wall compositional phenotypes support the proposition that (1) GAUT proteins play a role in wall biosynthesis, (2) GAUTs 6, 9,10, and 11, which have the highest amino acid similarity to GAUT1, have putative functions in pectin biosynthesis, and (3) GAUTs 13 and 14 are likely to have putative functions in xylan biosynthesis like GAUT12, or in pectin RG-I biosynthesis. The mutant wall composition phenotypes presented here are not sufficient to prove GAUT function, but serve to support hypotheses regarding GAUT function. The data demonstrate that mutants corresponding to more than half of the gaut mutants have significantly altered wall polysaccharides and strongly support a role for the family in pectin and/or xylan synthesis and function. Potential gene redundancy could explain the lack of wall phenotypic changes in some of the gaut mutants, and the generation of double mutants might uncover phenotypes masked by such potential redundancy.

### Example 2

### Materials and Methods

Plant Materials and Growth Conditions. Two independent T-DNA insertion lines (00091 and 02925) in GAUT14 were obtained from the Arabidopsis Biological Resource Center (www.biosci.ohio-state.edu/pcmb/Facilities/abrc/abrchome.htm). Arabidopsis WT (Arabidopsis thaliana var. Columbia S6000) and gaut14 mutant seeds were sown on pre-moistened soil in a growth chamber with 60% constant relative humidity with a photoperiod 14/10 light/dark cycle (14 h 19° C. and 10 h 19° C.) and fertilized as described (Example 1). The 7-weeks old WT and PCR-genotyped mutant plants were harvested used for glycome profiling and as a carbon source for bacterial growth analyses.

DNA Extraction, mutant genotyping and identification of two T-DNA insertion lines in GAUT14. Approximately 100 mg of leaf tissue was ground with a mortar and pestle to fine powder. The ground leaf tissue was suspended in 0.5 ml extraction buffer (100 mM EDTA pH 8.0, 100 mM Tris-HCl pH 8.0, 250 mM NaCl, 100 μg ml⁻¹ proteinase K and 1% (w/v) n-lauroylsarcosine). The suspension was extracted with an equal volume of phenoLchloroformn:isoamyl alcohol (49:50:1, v/v). DNase-free RNase A (1 μl) was used to degrade RNA for 20 min at 37° C. and the DNA was precipitated twice with 70% (v/v) ethanol.

The genotype of gaut14 mutant plants was determined by the appropriate GAUT14 gene-specific primer with T-DNA-specific primers based on the ability of the LB primers to anneal.

The GAUT14 gene-specific primer pairs used for genotyping were AtGAUT14 (forward, 5′-ATGCAGCTTCACATATCGCCTAGCATG (SEQ ID NO:160)′; reverse, 5′-CAGCAGATGAGACCACAACCGATGCAG (SEQ ID NO:161)). Following T-DNA-specific primer pairs were used for genotyping like gaut14-1 (forward, 5′-TTAAGTCTCCCTGGACAACTATATCAT (SEQ ID NO:162); reverse, 5′-CAATTGTCAAGTTGGTTTCTTTTCT(SEQ ID NO:163)), gaut14-2 (forward, 5′-TTGGGTCCGCTACTGATCTGA (SEQ ID NO:164); reverse 5′-GCAGTGATCCACTACAATGGGC (SEQ ID NO:165)). Homozygous lines were identified by PCR for further characterization of the gaut14 mutants. The two mutant lines are designated gaut14-1 and gaut14-2.

Quantitative Real-Time PCR. For expression analysis wild type, Arabidopsis leaf, flower, upper stem, middle stem, lower stem, hypocotyls, silique and seeds were harvested and frozen immediately in liquid nitrogen and stored at −80° C. until use. All the tissues were ground to a fine powder using N₂(l) in a chilled mortar and pestle. Total RNA was extracted using the RNeasy Plant Mini Kit (Qiagen) followed by DNAse (DNA-free kit, Ambion) treatment to remove genomic DNA contamination. First strand cDNA synthesis was performed using 1 μg of total RNA with a blend of oligo (dT) and random primers in the iScript™cDNA Synthesis Kit (Bio-Rad, Hercules, Calif., USA) according to the manufacturer's instructions. The primers used to amplify the GAUT14 transcripts of the above tissues were as follows: AtGAUT14 (forward, 5′-CAAGGCAGTCTGCAGATATTAC (SEQ ID NO:166); reverse, 5′-CTTATGCAACCTTCCCTTCG (SEQ ID NO:167)), with two primers (forward, 5′-AGTGTCTGGATCGGTGGTTC (SEQ ID NO:168); reverse, 5′-ATCATACTCGGCCTTGGAGA (SEQ ID NO:169)) to amplify the actin2 transcript were also designed as an internal standard for quantification. PCR reactions were performed in a 96-well plate with a Bio-Rad iCycler MyiQ Real-Time PCR Detection System. Detection of products was by binding of the fluorescent DNA dye SYBR Green (iQ SYBR Green Supermix) to the PCR products. All assays were carried out in triplicate, and one-set of no-template controls was included per gene amplification. A PCR reaction contained a total volume of 25 μl with appropriate cDNA, SYBR Green, and both forward and reverse primers. Thermal cycling conditions were as follows: initial activation step 3 min at 95° C., followed by 15 s at 95° C., 30 s at 55° C., 30s at 72° C. for 45 cycles, 1 min 95° C., 1 min 55° C., a melting curve program (80 cycles, 10 s each of 0.5° C. elevations starting at 55° C.) and a cooling step to 4° C. The presence of one product per gene was confirmed by analysis of the disassociation curves. The iCycler MyiQ software 1.0 (Bio-Rad, Hercules, Calif., USA) was used to calculate the first significant fluorescence signal above noise, the threshold cycle (Ct). The PCR efficiencies (E) of each amplicon were determined by using pooled cDNA originating from the assayed tissues in 4-fold serial dilutions and the calculation was performed in the iCycler MyiQ software 1.0 (Bio-Rad). The relative transcript levels (RTL) was calculated as follows: 100 000×E^{CT Control}/E^{CT Target}, thus normalizing target gene expression to the control gene expression.

Isolation of cell wall, cell wall (AIR) fractionation and ELISA assay. The walls from leaves and stem of WT and two gaut14 mutants were sequentially extracted from frozen ground tissue with 80% ethanol, 100 ml ethanol, chloroform:methanol (1:1) (Example 1) and the resulting AIR (alcohol insoluble residue) was washed with acetone. The cell walls (AIR) were then de-starched with alpha amylase (Sigma) in 50 mM ammonium formate, pH 6.5, for 24 hrs. In the next step the AIR walls were sequentially fractionated enzymatically and chemically. The enzyme treatments were carried out in ammonium formate, pH 6.0 for 24 hours at room temperature with Aspergillus niger EPG and Aspergillus niger PME. The walls were then sequentially extracted with 50 mM sodium carbonate (pH 10.0) and then with 1M KOH and 4M KOH. Each fraction was neutralized (if necessary), dialyzed and lyophilized for analysis. The extracted cell walls were dissolved in deionized water (0.2 mg/mL) and the total amount of sugar measured. Equal amounts of sugar (500 ng) were applied to the wells of ELISA plates (Costar 3598) and a series of 152 monoclonal antibodies directed against plant cell wall carbohydrate epitopes were used for this analysis. The data are presented as a heat map on a hierarchical clustering (Pattathil et al., 2010, Plant Physiol., 153:514-525).

Microorganisms and bacteria growth medium in WT and gaut14-1 and gaut14-2 mutants in Arabidopsis.

Microorganisms: Caldicellulosiruptor bescii DSM 6725 (former Anaerocellum thermophilum DSM 6725) was obtained from the DSMZ (http://www.dsmz.de/index.htm). Caldicellulosiruptor saccharolyticus DSM 8903 was a gift from Robert Kelly of North Carolina State University.

Growth medium. C. bescii DSM 6725 and C. saccharolyticus DSM 8903 were grown in the 516 medium (Svetlichnyi et al., 1990, Microbiology (Translation of Mikrobiologia) 59:598-604) except that vitamin and trace mineral solutions were modified as follows. The minerals solution contained per liter: NH₄Cl0.33 g, KH₂PO₄ 0.33 g, KCl 0.33 g, MgCl₂×6 H₂O 0.33 g, CaCl₂×2 H₂O 0.33 g, yeast extract 0.5 g, resazurin 0.5 mg, vitamin solution 5 ml, trace minerals solution 1 ml. The vitamin solution contained (mg/l): biotin 4, folic acid 4, pyridoxine-HCl 20, thiamine-HCl 10, riboflavin 10, nicotinic acid 10, calcium panthotenate 10, vitamin B₁₂ 0.2, p-aminobenzoic acid 10, lipoic acid 10. The trace minerals solution contained (g/l) FeCl₃ 2, ZnCl₂ 0.05, MnCl₂×4H₂O 0.05, H₃BO₃ 0.05, CoC₂×6H₂O 0.05, CuCl₂×2H₂O 0.03, NiCl₂×6H₂O 0.05, Na₄EDTA (tetrasodium salt) 0.5, (NH₄)₂MoO₄ 0.05, AlK(SO₄)₂.12H₂O 0.05. The medium was prepared anaerobically under a N₂/CO₂ (80:20) atmosphere, NaHCO₃ (1 g/l) was added and it was reduced using (per liter) 0.5 g cysteine and 0.5g N₂S. Finally, 1 ml/L of 1M potassium phosphate buffer (pH 7.2) was added. The final pH was 7.2. The medium was filter-sterilized using a 0.22 micron pore size sterile filter (Millipore Filter. Corp., Bedford, Mass.). Arabidopsis (wild type and two gaut14 mutants) dried stems were used as a growth substrate at a final concentration of 0.5% (wt/vol). The dried intact biomass was added directly to each bottle. Growth was at 78° C. (A. thermophilum) or at 71° C. (C. saccharolyticus) as static cultures in 50 ml serum bottles with 20 ml medium with shaking (150 rpm) for 24 hours. The culture media containing the insoluble substrates without inoculation were used as controls. All growth experiments were run in triplicate. Cell density was monitored by cell count using phase-contrast microscope with 40× magnification and expressed as cells per ml. Samples of growing cultures were taken each three hours and cell count was done immediately.

### Results

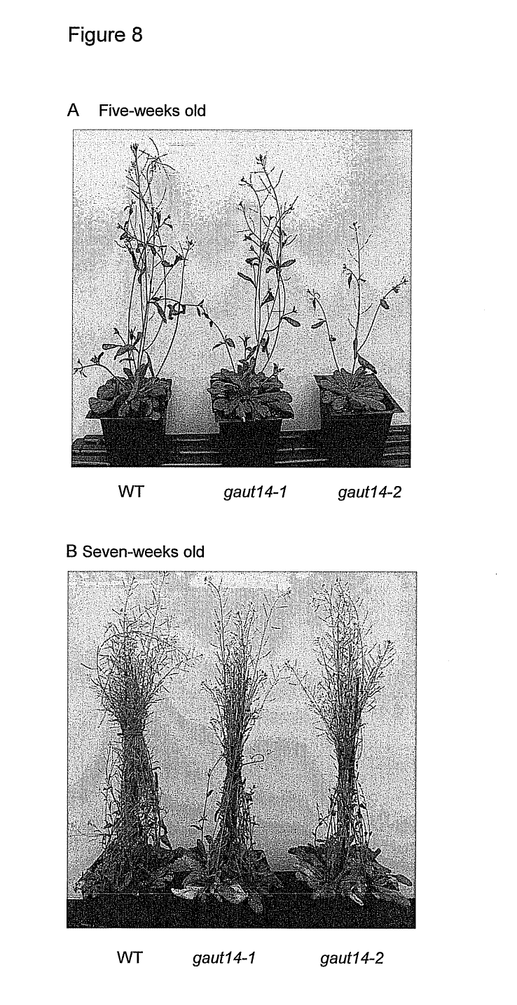

Endogenous expression of GAUT14 in Arabidopsis. The level of GAUT14 transcripts in various WT tissues was investigated using qRT PCR as described in the materials and methods. Acting used as a control. GAUT14 mRNA was detected in stem, leaf, flower, hypocotyl, silique and seeds in all major tissues, suggesting a role in plant growth and development (FIG. 8). However, transcript expression was more prominent in upper and lower stem in Arabidopsis.

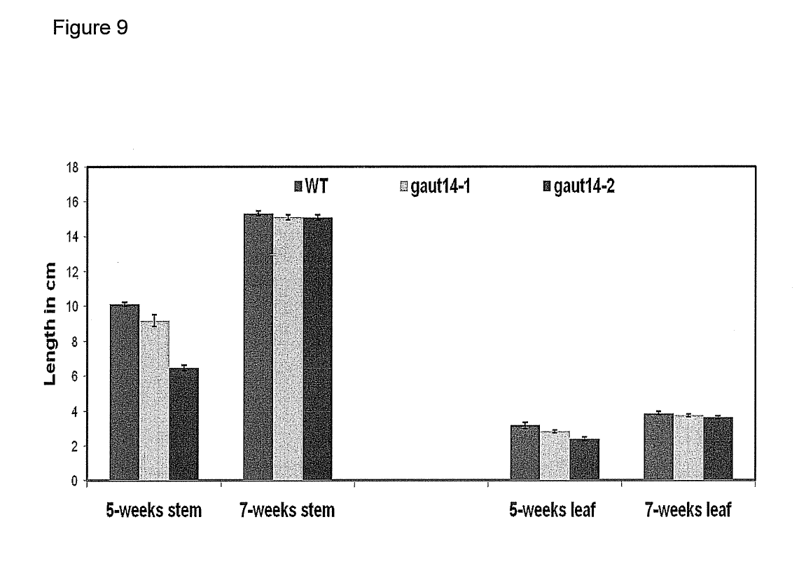

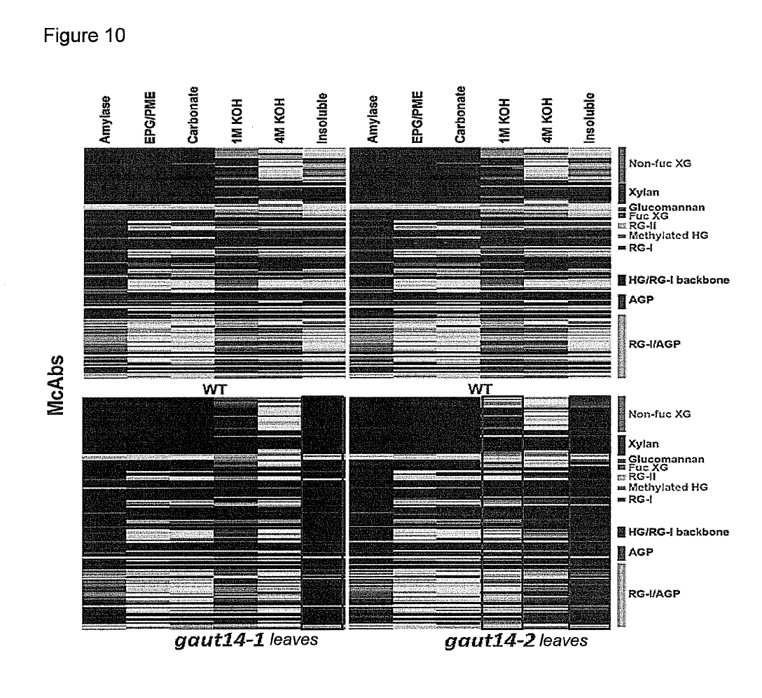

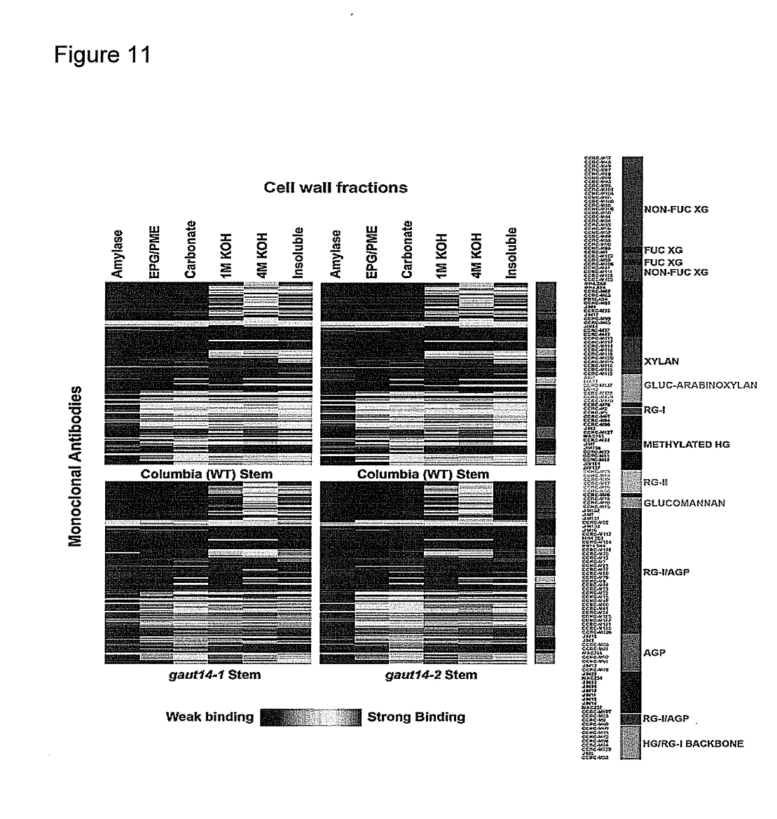

Position of T-DNA insertion, phenotypes and growth measurement of T-DNA knock-out mutants in gaut14-1 and gaut14-2. The two T-DNA insertional mutants for GAUT14 (At5g15470) were obtained from the Salk collection as described in materials and methods. The T-DNA is inserted in the fourth exon in gaut14-1 (Salk_{—}000091) and in the 3′ untranslated region (UTR) in gaut14-2 (Salk_{—}029525) mutants (FIG. 9). Five week old homozygous gaut14 mutants exhibited a clear visible phenotype when grown on soil, with reduced stem length and leaf blade length (FIG. 10). There is a 10% and 36% decrease in stem length in gaut14-1 and gaut14-2 mutants, respectively in comparison to their wild type plants (FIG. 11). Similarly there is a 10% and 24% decrease in leaf blade length in gaut14-1 and gaut14-2 mutants, respectively (FIG. 11). Interestingly, the reduced growth phenotype in these two gaut14 mutants caught up to WT within 7-weeks.

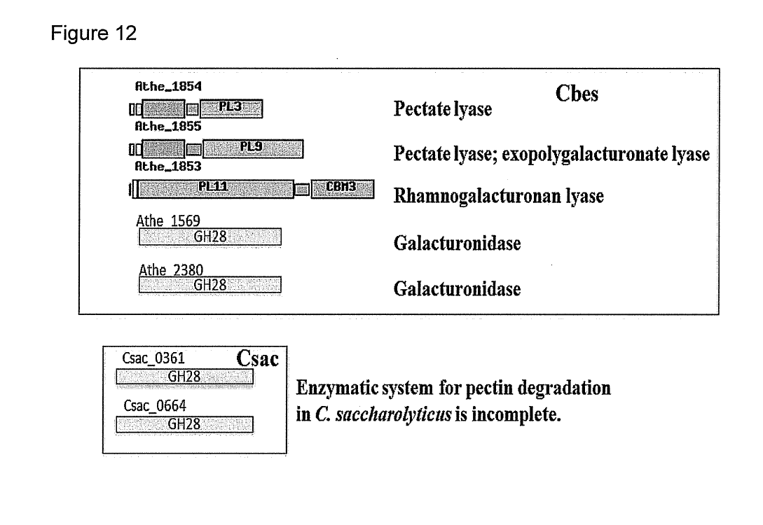

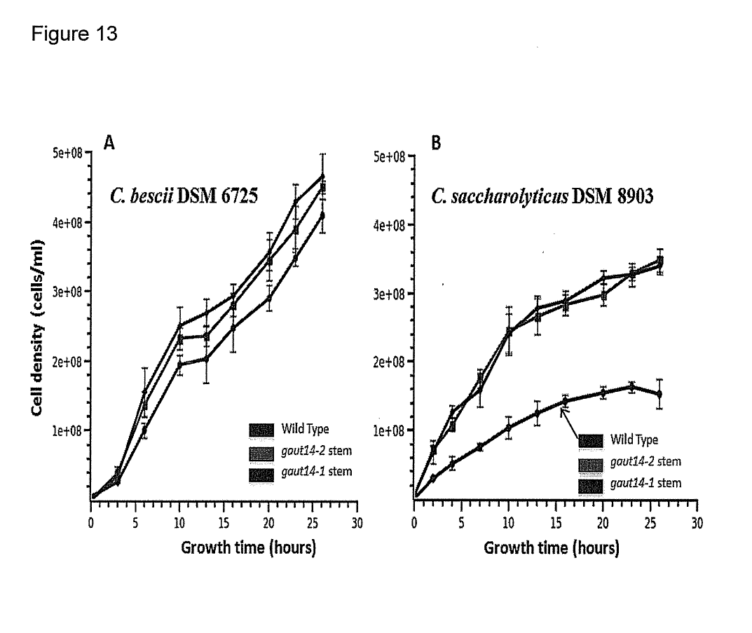

Glycome profile of WT and gaut14 mutants in Arabidopsis. A method recently developed by Pattathil et al. (2010, Plant Physiol., 153:514-525) was used to determine how the release of sequentially extracted cell wall polymers from the stem and leaf cell walls of WT are different from those of the gaut14 mutants based on detection of released wall material using 150 cell wall carbohydrate-directed monoclonal antibodies. Both the gaut14 mutant leaf walls retain less polysaccharide in the insoluble pellet in comparison to the WT leaves (FIG. 12). The release of more cell wall polymers was detected in the 4M KOH fractions in gaut14-1 than WT, especially in the case of RG-I/AGP directed antibodies. However, more significant differences were exhibited by gaut14-2 mutants with more release of polysaccharides in the early stages of fractionation, for example in the 1M KOH fraction (FIG. 12). The same pattern of less polysaccharide material being retained in the insoluble pellet of the gaut14-1 and gaut14-2 mutant stem was obtained (FIG. 13). The EPG/PME and carbonate fractions in gaut14-1 showed different binding patterns from WT, especially in the case of HG/RG-I backbone, AGP and RG-I/AGP directed antibodies (FIG. 13). The glycome profiles suggest that the absence of GAUT14 products have profound effects on the cell wall extractability which makes the wall more easily extractable.

Growth of two pectin degrading bacteria in Arabidopsis WT and gaut14 mutants. Growth of Caldicellulosiruptor bescii DSM 6725 was quite efficient on Arabidopsis wild type and on the gaut14-1 and gaut14-2 mutants (FIG. 15). After 24 hours, the cultures were still growing, although they reached middle stationary phase. Cell densities upon growth on Arabidopsis WT, gaut14-1 and gaut14-2 mutants were over >4e+8 with slightly at 26 hours. C. bescii grew somewhat better on the Arabidopsis gaut14 mutants than on the Arabidopsis WT. Growth of C. saccharolyticus DSM 8903 on Arabidopsis WT, gaut14-1 and gaut14-2 mutants was much different than the growth of C. bescii on the same walls (FIG. 15). The bacterium grew less well on WT, and grew better on the two gaut14 mutants, approaching stationary phase growth after 24 hours. The growth was more efficient on the gaut14-1 and gaut14-2 mutants than on the WT, with final cell densities of 3.5e+8, 3.4e+8 and 1.6e+8 cells/ml, respectively.

### Discussion

C. bescii and C. saccharolyticus are thermophilic anaerobic bacteria capable of growing on different polysaccharides including crystalline cellulose, xylans, starch and pectin (Rainey et al., 1994, FEMS Microbiol Lett 120: 263-266; Yang et al., 2009, Appl. Environ. Microbiol., 75:4762-4769). The genome of C. saccharolyticus has been available for about three years. The genome of C. bescii was sequenced and analyzed recently (Kataeva et al., 2009, J. Bacteriol., 191: 3760-3761). Both genomes are very similar and encode sets of enzymes acting on polysaccharides and metabolizing multiple sugars. Both bacteria are able to process cellulose and xylan simultaneously and grow on Arabidopsis plant biomass. However, comparison of the growth of C. bescii and C. saccharolyticus on Arabidopsis WT and on the gaut14 knockouts mutants, mutants that appear to modify the pectin biosynthesis pathway, revealed differences. In particular, C. bescii grew well on all Arabidopsis samples but showed somewhat better growth on the gaut14 mutants with a final cell density exceeding 4e+8 cells/ml, which is a high density for anaerobic thermophiles. C. saccharolyticus also grew on the three different Arabidopsis biomass sources, however, the cells reached stationary phase in shorter time and the cell densities were lower for C. saccharolyticus compared to C. bescii. Moreover, the growth of C. saccharolyticus on WT Arabidopsis biomass was much less efficient compared to growth on gaut14-1 and gaut14-2 mutant biomass, with lower final cell densities when grown on WT.

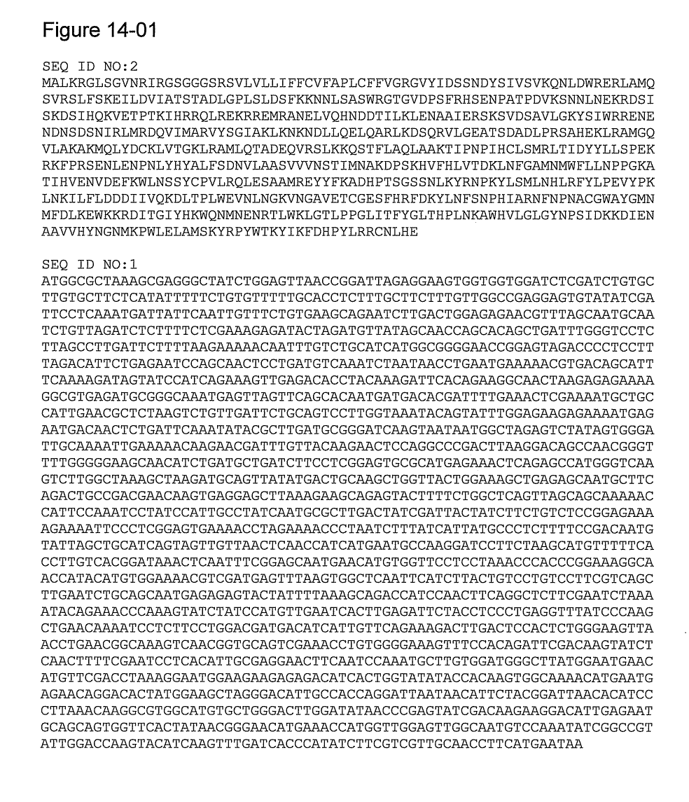

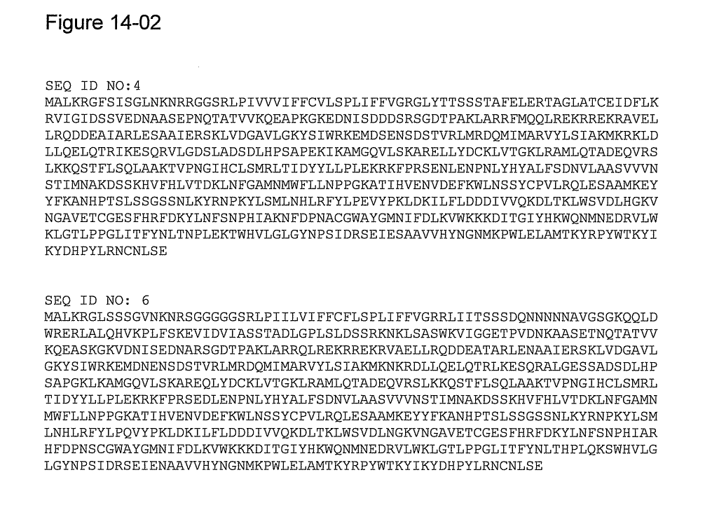

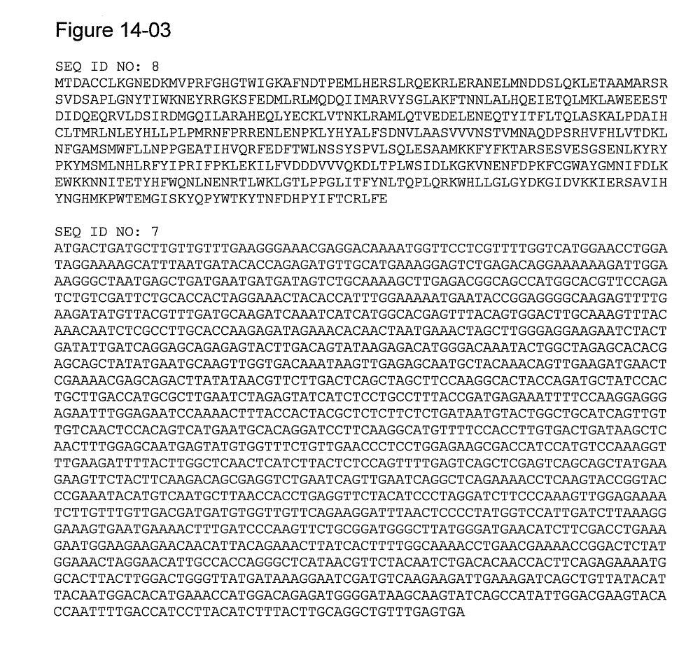

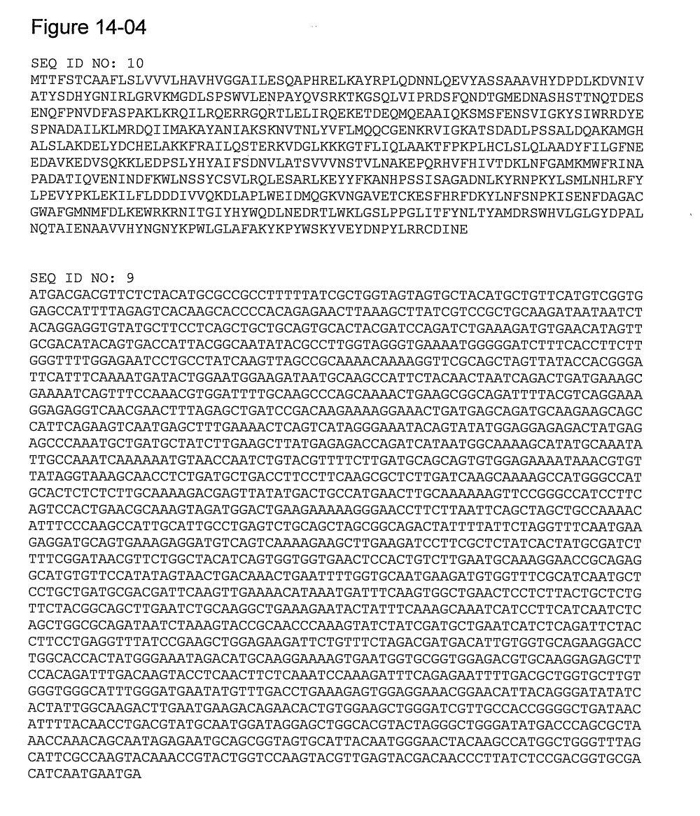

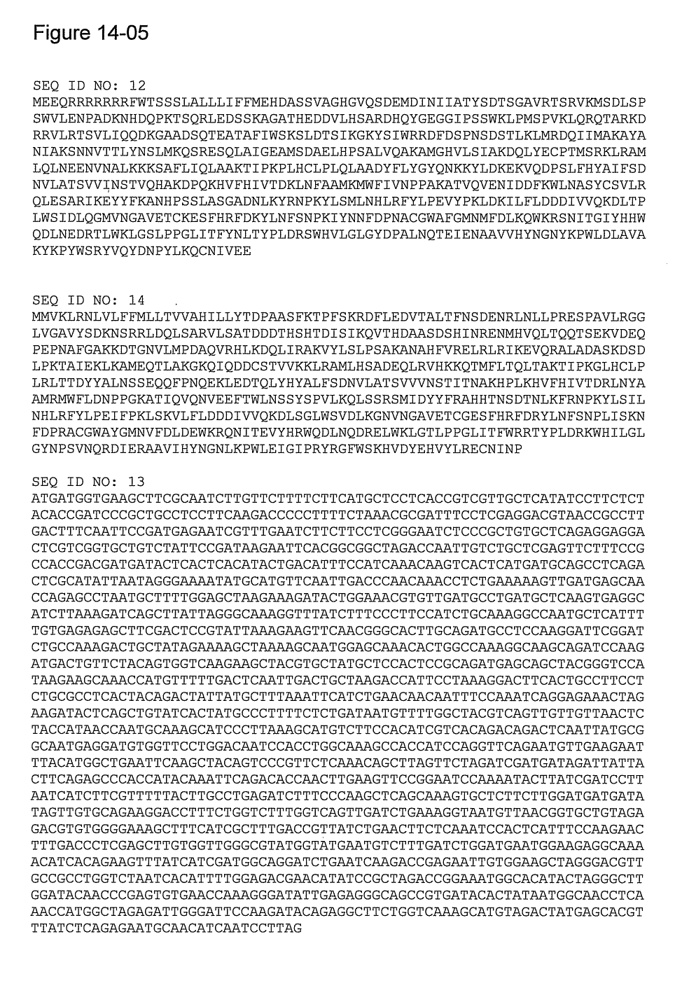

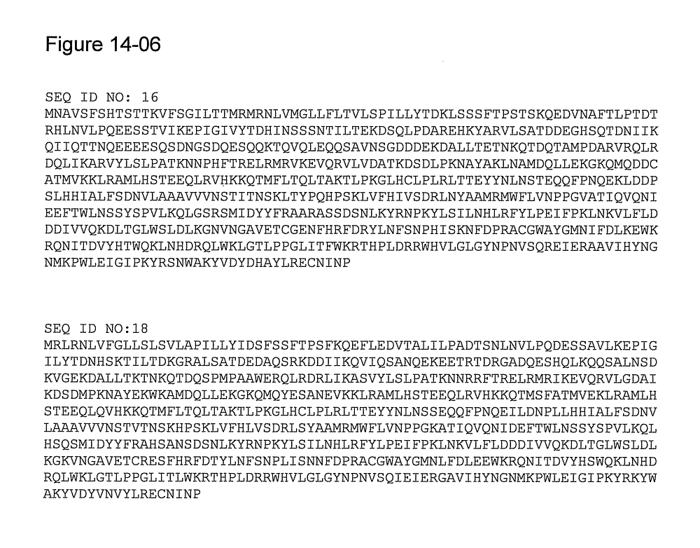

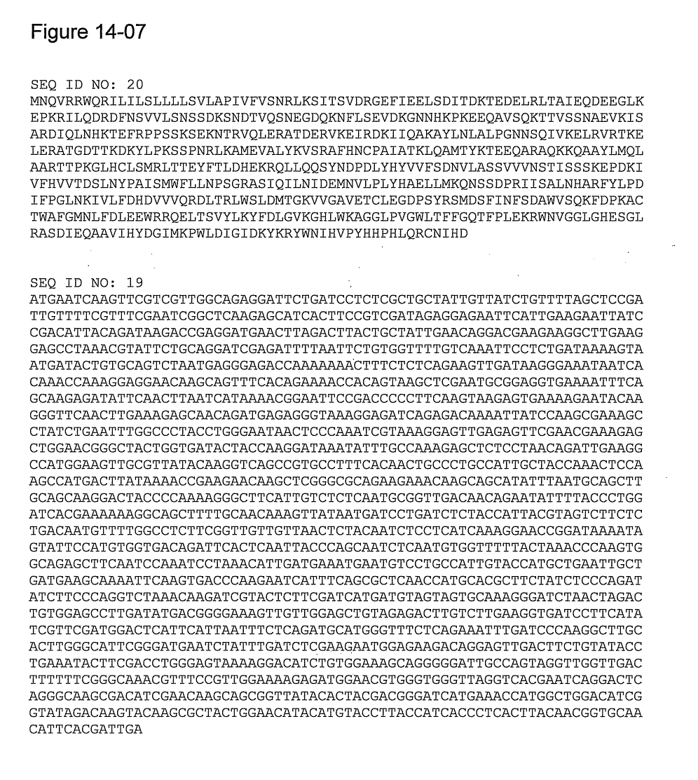

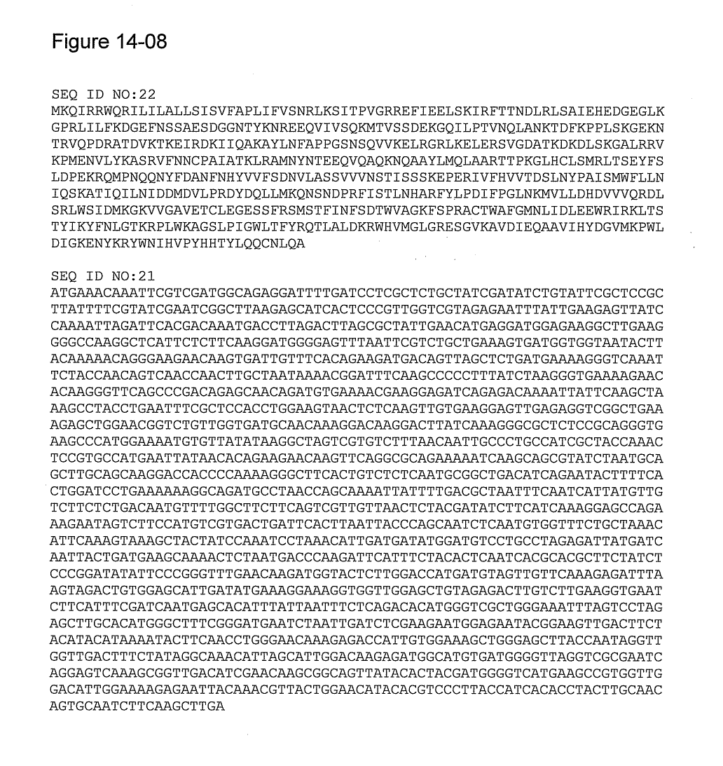

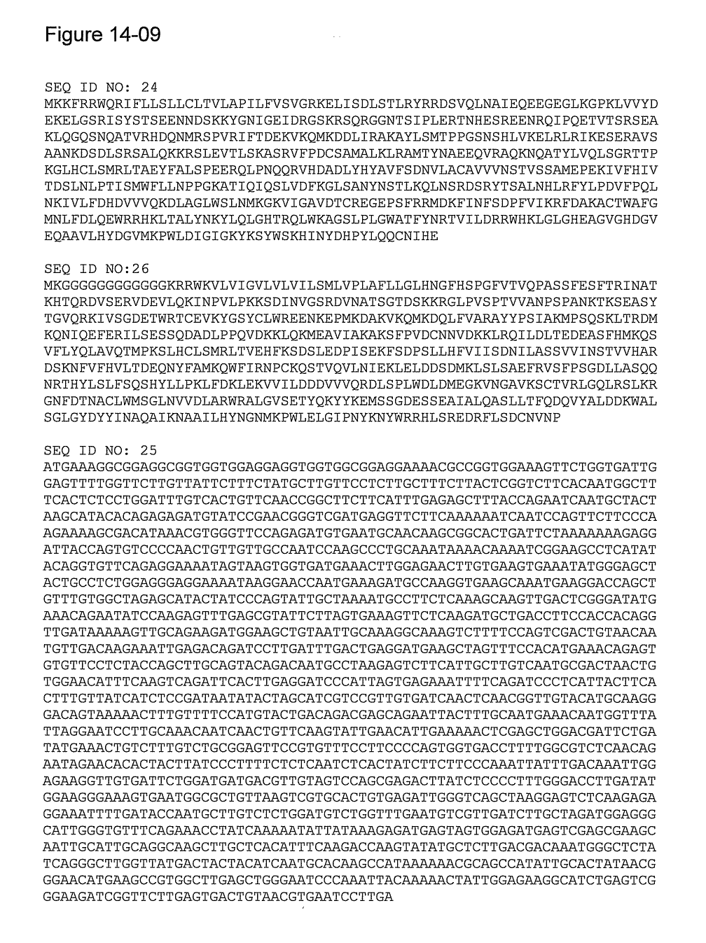

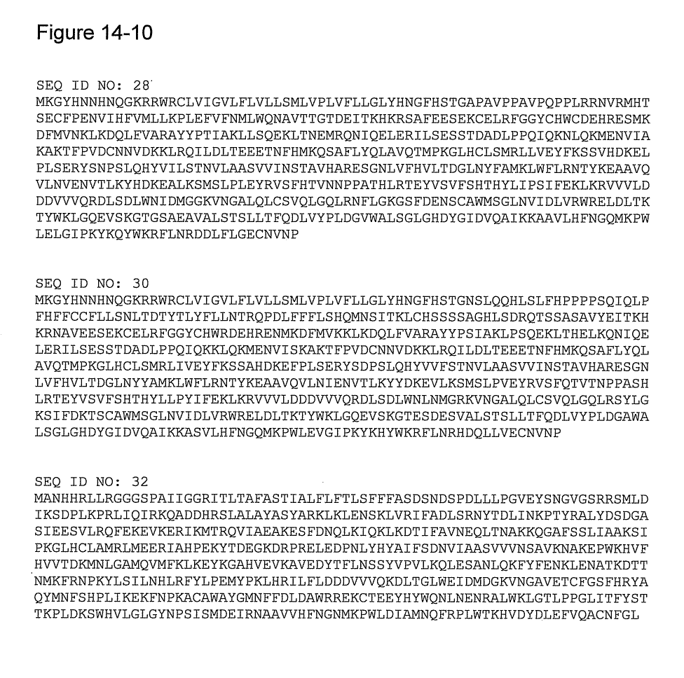

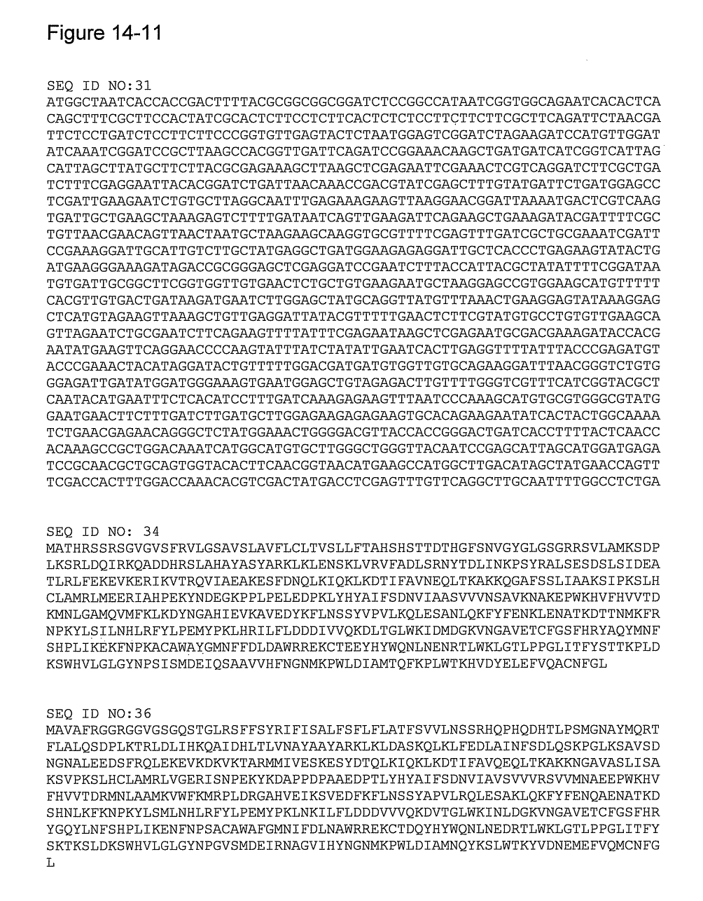

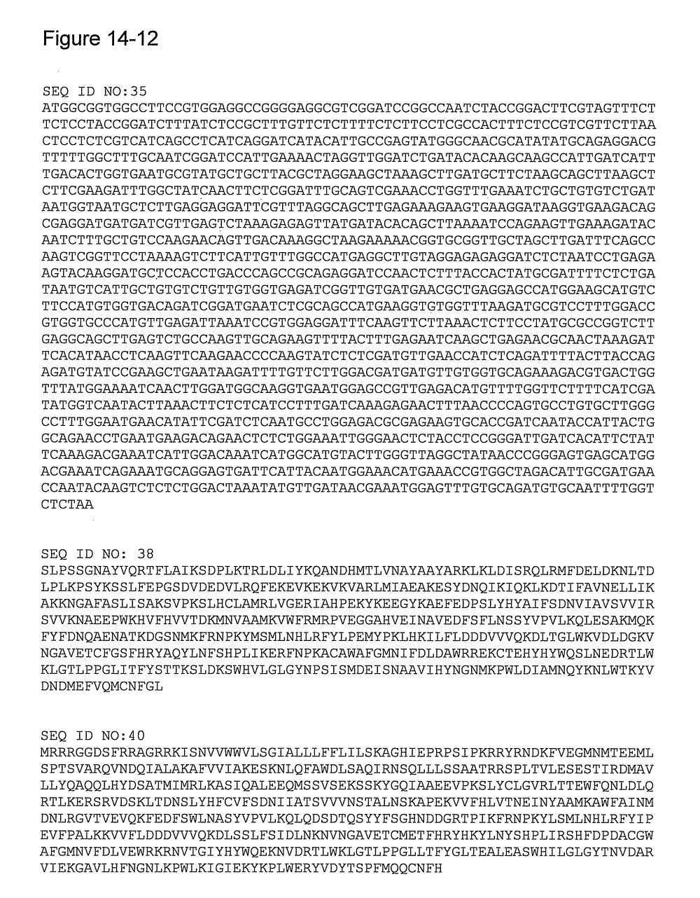

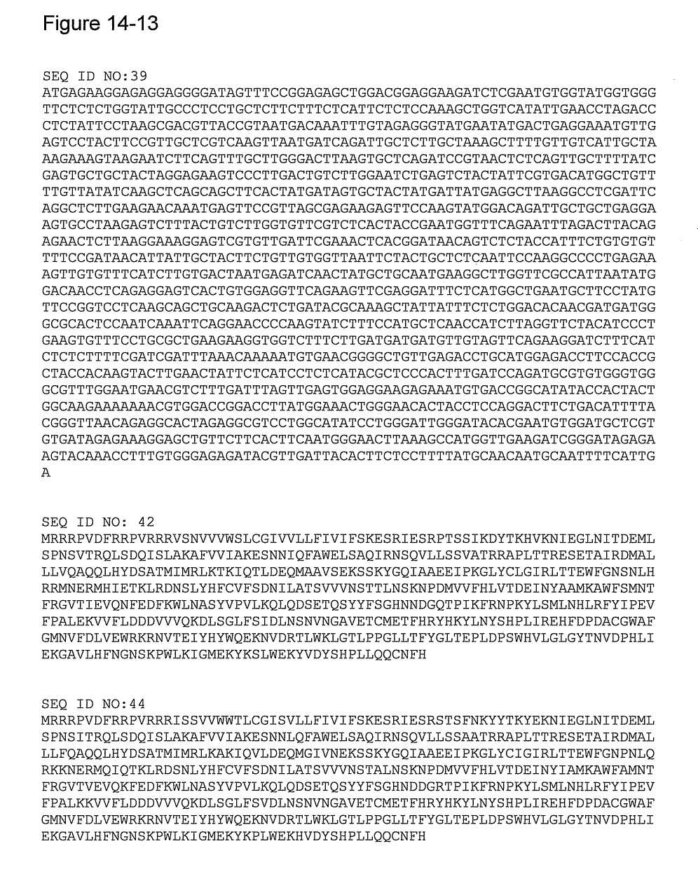

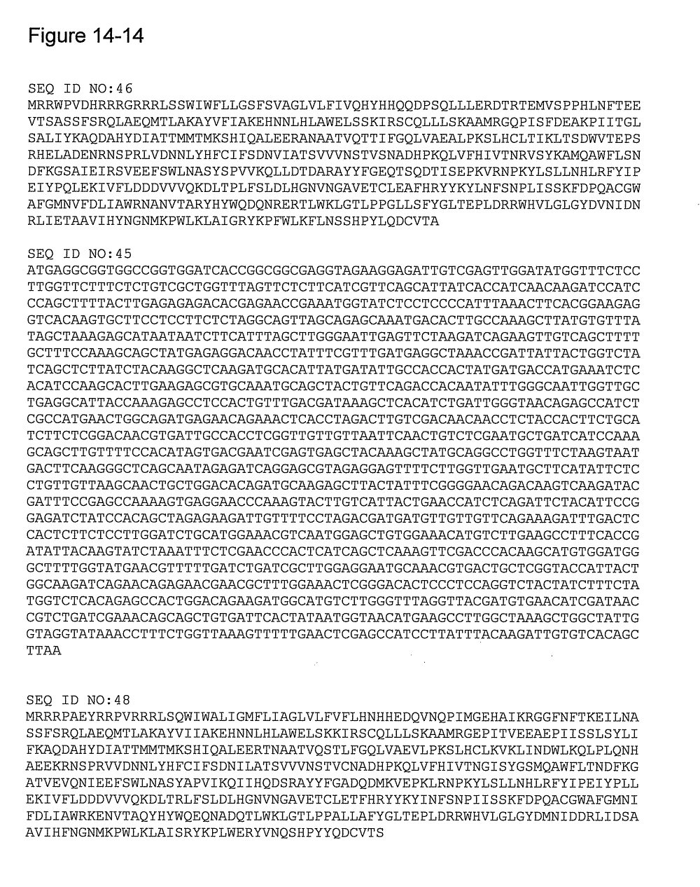

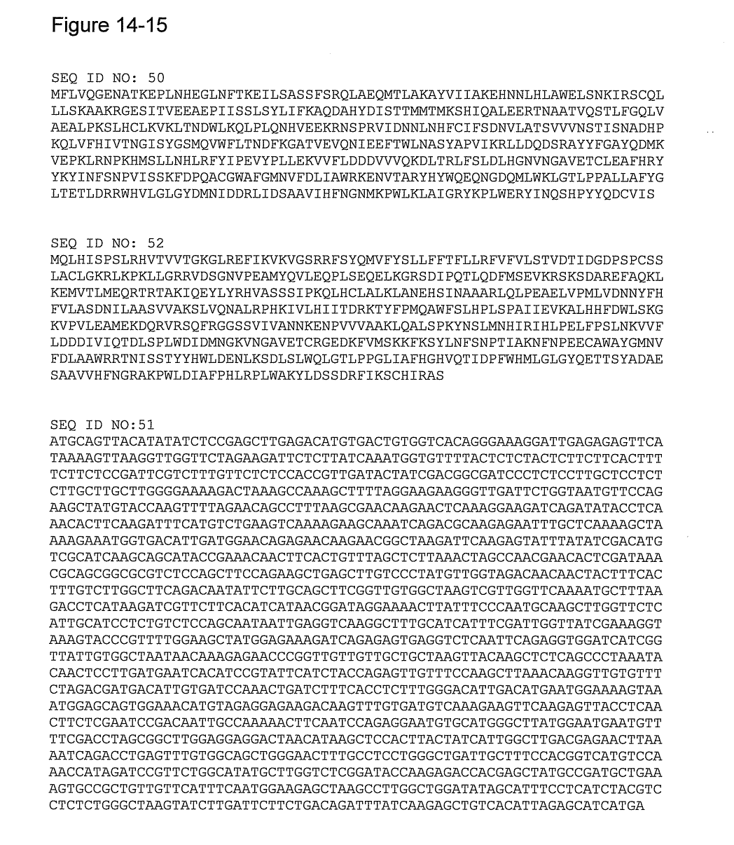

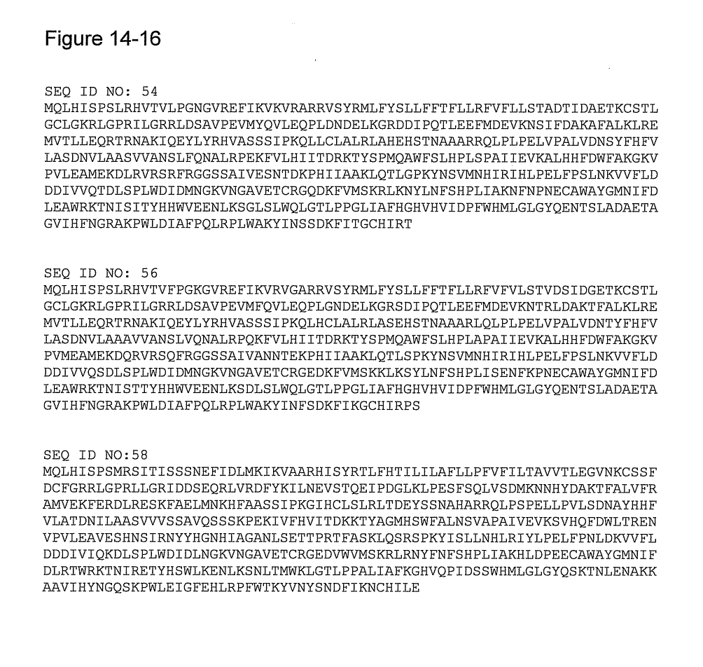

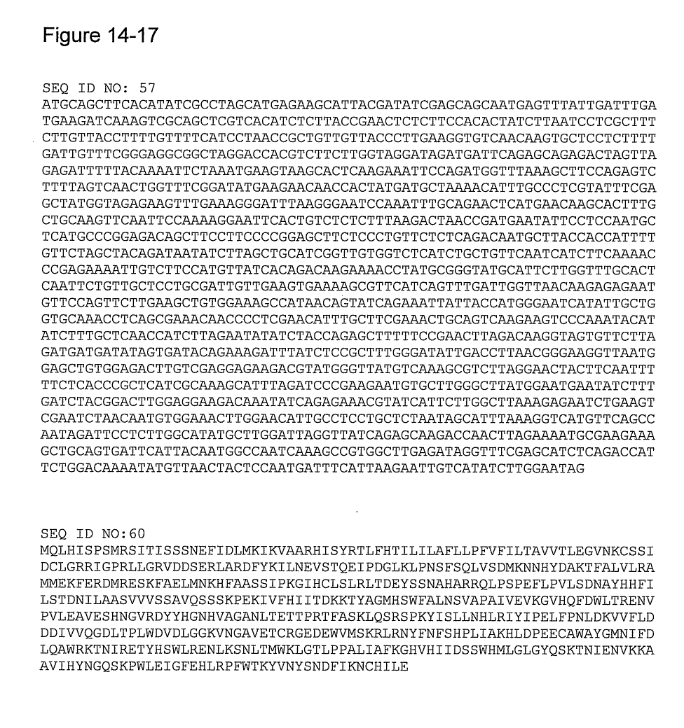

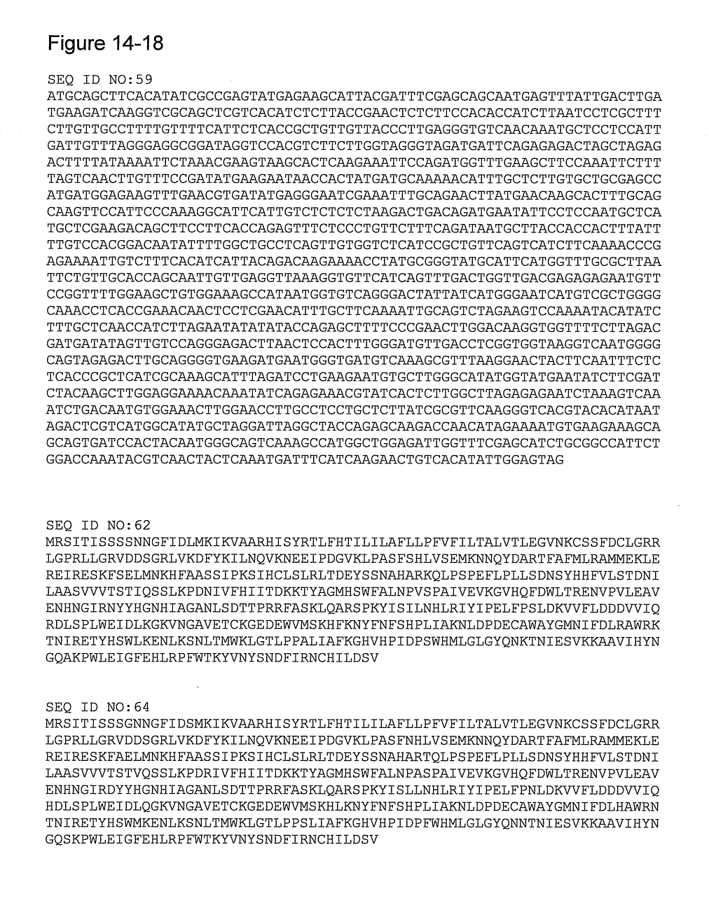

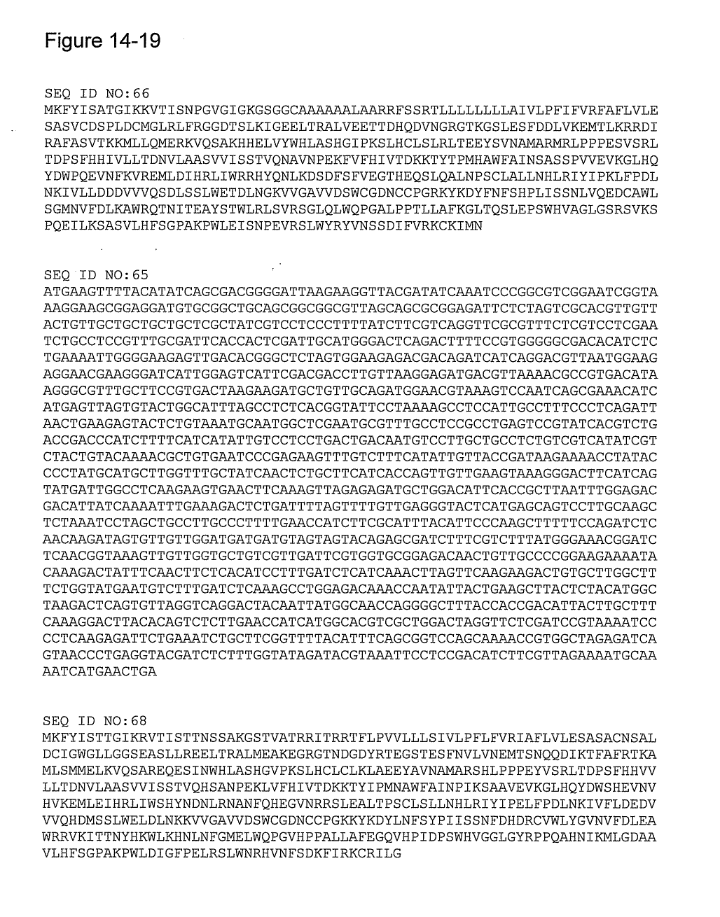

These differences could be attributed to the different pectin degrading systems produced by these bacteria (FIGS. 14A and 14B). C. bescii has a unique enzymatic system related to pectin degradation. It is composed of 3 polysaccharide lyases (PL) of different PL families (encoded by Cbes_{—}1853 -1855 genes). In addition, the genome encodes two glycoside hydrolases of family 28 (GH28, see CAZy database) capable of hydrolysis of unsubstituted polygalacturonic acid as part of pectin backbone (FIG. 13A). Search within 25 genomes of anaerobic thermophilic bacteria (our data, not published) revealed that only two of them encode sets of 3 PLs of different families (C. bescii and Cl. thermocellum, although the latter does not encode GH28s). In contrast to C. bescii, all PLs are missing from the genome of C. saccharolyticus. The genome encodes only two GH28s with limited activity against pectin (FIG. 13B). This genome analysis suggests that better growth of C. bescii on Arabidopsis vs. C. saccharolyticus is related to a comprehensive set of pectin degrading enzymes while C. saccharolyticus has a truncated set composed of just two GH28s. The gaut14-1 and gaut14-2 mutants have either less content of pectin or modified pectin. As a result, C. saccharolyticus grows better on the mutants than on WT Arabidopsis without interruptions in pectin content/structure.

The present data suggest that the pectin, similar to lignin, is a “recalcitrance factor” of plant biomass decreasing accessibility of cellulose and hemicelluloses to the corresponding degrading enzymes. The data also are very promising for the development a novel approach to test recalcitrance of plant biomass. This “microbial recalcitrance test” would be based on a limited ability of a given microorganism to degrade a particular constituent(s) of plant biomass, so that genetically modified plants with the decreased amounts of, or simplified structures of, the relevant wall polymer will serve as better growth substrates in comparison to wild type plants.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for using a transgenic plant, the method comprising processing a transgenic plant to result in pulp, wherein the transgenic plant comprises decreased expression of a coding region encoding a GAUT polypeptide compared to a control plant.

2. The method of claim 2 wherein the processing comprises a physical pretreatment, a chemical pretreatment, or a combination thereof.

3. The method of claim 1 further comprising hydrolyzing the processed pulp.

4. The method of claim 1 further comprising contacting the processed pulp with an ethanologenic microbe.

5. The method of claim 4 wherein the ethanologenic microbe is a eukaryote.

6. The method of claim 1 further comprising obtaining a metabolic product.

7. The method of claim 6 wherein the metabolic product comprises ethanol.

8. The pulp of claim 1.

9. A method comprising hydrolyzing a pulp, wherein the pulp comprises cells of a transgenic plant, wherein the cells comprise a mutation in a coding region encoding a GAUT polypeptide.

10. The method of claim 9 wherein the hydrolyzing comprises contacting the pulp with a composition comprising a cellulase under conditions suitable for hydrolysis.

11. The method of claim 9 further comprising contacting the hydrolyzed pulp with an ethanologenic microbe.

12. The method of claim 11 wherein the ethanologenic microbe is a eukaryote.

13. The method of claim 9 further comprising obtaining a metabolic product.

14. The method of claim 13 wherein the metabolic product comprises ethanol.

15. A method for producing a metabolic product comprising:
contacting under conditions suitable for the production of a metabolic product a microbe with a composition comprising a pulp obtained from a transgenic plant, wherein the transgenic plant comprises decreased expression of a coding region encoding a GAUT polypeptide compared to a control plant.

16. The method of claim 15 wherein the microbe is an ethanologenic microbe.

17. The method of claim 16 wherein the ethanologenic microbe is a eukaryote.

18. The method of claim 15 further comprising obtaining a metabolic product.

19. The method of claim 15 wherein the metabolic product comprises ethanol.

20. The method of claim 15 wherein the contacting comprises fermenting the pulp.

21. The method of claim 20 wherein the fermenting comprises a simultaneous saccharification and fermentation.

22. The method of claim 1, 9, or 15 wherein the GAUT polypeptide is selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide.

23. A method for generating a transgenic plant having decreased recalcitrance, reduced lignification, increased growth, or the combination thereof, compared to a plant of substantially the same genetic background grown under the same conditions, the method comprising:
transforming a cell of a plant with a polynucleotide to obtain a recombinant plant cell;
generating a transgenic plant from the recombinant plant cell, wherein the transgenic plant has decreased expression of a coding region encoding a GAUT polypeptide compared to a control plant.

24. The method of claim 23 wherein the transgenic plant comprises a phenotype selected from decreased recalcitrance, reduced lignification, increased growth, or the combination thereof, compared to a control plant.

25. The method of claim 23 wherein the transgenic plant is a dicot plant.

26. The method of claim 23 wherein the transgenic plant is a monocot plant.

27. The method of claim 23 further comprising breeding the transgenic plant with a second plant, wherein the second plant is transgenic or nontransgenic.

28. The method of claim 23 wherein increased growth is selected from increased height or increased diameter.

29. The method of claim 23 wherein the transgenic plant is a woody plant.

30. The method of claim 29 wherein the transgenic plant is a member of the genus Populus.

32. The method of claim 23 further comprising screening the transgenic plant for decreased recalcitrance, reduced lignification, increased growth, or the combination thereof.

33. The method of claim 23 wherein the GAUT polypeptide is selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide.

34. A transgenic plant comprising decreased expression of a coding region encoding a GAUT polypeptide compared to a control plant.

35. The transgenic plant of claim 34 wherein the GAUT polypeptide is selected from a GAUT1 polypeptide, a GAUT2 polypeptide, a GAUT3 polypeptide, a GAUT4 polypeptide, a GAUT5 polypeptide, a GAUT6 polypeptide, a GAUT7 polypeptide, a GAUT8 polypeptide, a GAUT9 polypeptide, a GAUT10 polypeptide, a GAUT11 polypeptide, a GAUT12 polypeptide, a GAUT13 polypeptide, a GAUT14 polypeptide, or a GAUT15 polypeptide.

36. The transgenic plant of claim 34 wherein the GAUT polypeptide is selected from:
a polypeptide having an amino acid sequence that has at least 80% sequence identity with SEQ ID NO: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO: 22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, and SEQ ID NO:66.

37. The transgenic plant of claim 34 wherein the transgenic plant comprises a phenotype selected from decreased recalcitrance, reduced lignification, increased growth, or the combination thereof.

38. The transgenic plant of claim 34 wherein the transgenic plant is a dicot plant.

39. The transgenic plant of claim 34 wherein the transgenic plant is a monocot plant.

40. A part of the transgenic plant of claim 34 wherein the part is chosen from a leaf, a stem, a flower, an ovary, a fruit, a seed, and a callus.

41. The progeny of the transgenic plant of claim 34.

42. The progeny of claim 41 wherein said progeny is a hybrid plant.

43. A wood obtained from the transgenic plant of claim 34.

44. A wood pulp obtained from the transgenic plant of claim 34.

45. A method for using the plant of claim 34 comprising exposing material obtained from the plant to conditions suitable for the production of a metabolic product.

46. The method of claim 45 wherein the exposing comprises contacting the material with an ethanologenic microbe.

47. A method for measuring a change in recalcitrance of a plant comprising:
growing under suitable conditions a Caldicellulosiruptor saccharolyticus on material obtained from a first plant and a second plant, wherein the first plant is a transgenic plant of claim 7, and wherein the second plant is a control plant;
measuring (i) the time required for the C. saccharolyticus to reach stationary phase or (ii) the cell density after stationary phase is reached, wherein the C. saccharolyticus reaching stationary phase in shorter time or achieving a higher cell density when grown on the transgenic plant material indicates the transgenic plant has decreased recalcitrance compared to the control plant.
